(19) United States
(12) Patent Application Publication
(10) Pub. No.: US 2024/0035012 A1
(43) Pub. Date: Feb. 1, 2024

(54) ENGINEERED ACE2 OLIGOMERS AND USES THEREOF

(71) Applicant: Westlake University, Zhejiang (CN)

(72) Inventors: Bobo Dang, Zhejiang (CN); Liang Guo, Zhejiang (CN); Wenwen Bi, Zhejiang (CN); Mengjiao Li, Zhejiang (CN)

(21) Appl. No.: 18/022,467

(22) PCT Filed: Aug. 23, 2021
(86) PCT No.: PCT/CN2021/113962

§ 371 Date: Feb. 21, 2023

(30) Foreign Application Priority Data
Aug. 21, 2020 (WO) PCT/CN2020/110613

(51) Int. Cl. C12N 9/48; G01N 33/569; A61K 39/215; A61P 31/14

(52) U.S. Cl. CPC C12N 9/485; C12Y 304/17023; G01N 33/56983; A61K 39/215; A61P 31/14; G01N 2333/165; G01N 2333/948

(57) ABSTRACT

Provided are engineered ACE2 oligomers and compositions comprising the oligomers. Also provided are compositions and methods for treating or preventing coronavirus infection and detecting coronavirus.

### FIELD OF THE INVENTION

The present invention relates to engineered ACE2 oligomers and composition comprising the oligomers. The present invention also relates to compositions and methods for preventing or treating coronavirus infection and detecting coronavirus.

### BACKGROUND OF THE INVENTION

Coronavirus disease 2019 (COVID-19) caused by SARS-CoV-2 has resulted in a severe global pandemic. Following SARS-CoV, SARS-CoV-2 is yet another beta-coronavirus emerged to threaten human health. SARS-CoV-2 and SARS-CoV are very similar, sharing 79.5% sequence identity (1), having similar spike protein structures (2-4), and having the same cell surface receptor angiotensin converting enzyme II (ACE2) (1, 5). Unfortunately, seventeen years after severe acute respiratory syndrome (SARS) pandemic, no targeted vaccines or therapeutics were approved for SARS which would have a high probability to treat COVID-19. Many neutralizing antibodies against SARS-CoV-2 are currently being urgently developed (6-10), some of these might become available later this year or next year. However, RNA viruses are known to have higher mutation rates (11, 12), mutation strains making current SARS-CoV-2 neutralizing antibodies ineffective could develop in the future, and many SARS-CoV-2 mutations have already been identified such as D614G(13-16). The appearance of COVID-19 after SARS indicates other related coronavirus pandemic will likely happen in the future too. Thus, therapeutics that are broadly effective against SARS-CoV-2 and mutants, even other SARS-CoV-2 related coronaviruses are highly desirable. Both SARS-CoV-2 and SARS-CoV bind ACE2 for cell entry, SARS-CoV-2 mutants and future related coronavirus will likely bind ACE2 for infection too. Therefore, decoys proteins engineered based on ACE2 could serve as the most broadly neutralizing proteins against these viruses and will be least likely to face mutational escape.

Furthermore, ACE2 biological function supports using ACE2 decoy proteins for SARS-CoVs infection treatment. Coronavirus infection or even spike protein binding can cause shedding of ACE2 from cell surface resulting decreased ACE2 expression level and accumulation of plasma angiotensin II (17-19) and this is closely related with acute lung injury(17, 20-22). Replenishing soluble ACE2 could alleviate acute respiratory distress syndrome (ARDS) (17, 21-23). In fact, it has been shown ACE2 peptidase domain could inhibit SARS-CoVs infection in cell assays and organoids (24-26), one clinical trial(NCT04335136) was also registered to use recombinant ACE2 to treat COVID-19. However, recombinant soluble ACE2 only has moderate binding affinity to SARS-CoV-2 spike protein (˜30 nM) (27) and can only inhibit virus at high concentration(24, 26, 28, 29), thus it may not be an optimal molecule to inhibit virus infection. Engineered ACE2 bearing multiple mutations and dimeric ACE2-ig have been shown to have better inhibition activities (25, 28-30). Spike proteins of SARS-CoVs function as trimers (2-4), we envisioned an engineered trimeric ACE2 protein could potentially bind up to three receptor binding domains (RBD) on spike protein to drastically increase binding affinity through avidity effect and to potently inhibit SARS-CoVs.

SARS-CoV-2 enters cells via ACE-2, which binds the trimeric spike protein with moderate affinity (K_{D}˜30 nM). Despite a constant background mutational rate, the virus must retain binding with ACE2 for infectivity, providing a functional constraint for SARS-CoV-2 inhibitors. We engineered a trimeric ACE2 (T-ACE2) that binds spike protein with extremely high affinity (K_{D}<1 pM), while retaining ACE2 native sequence. T-ACE2 can potently neutralize SARS-CoV-2, SARS-CoV, eight SARS-CoV-2 mutants and a SARSr-CoV tested. The cryo-EM structure of the complex revealed T-ACE2 can induce spike protein to transit to three RBDs up conformation for binding. We believe T-ACE2 represents a valuable approach for developing broadly neutralizing proteins against SARS-CoVs and mutants.

### SUMMARY OF THE INVENTION

In one aspect, the present inventions provide ACE2 oligomers, wherein the ACE2 oligomer is formed by monomers, and each monomer comprises a soluble ACE2, a linker and an oligomerization motif. In some embodiments, the monomer comprises from N-terminal to C-terminal a soluble ACE2, a linker and an oligomerization motif. In some embodiments, the ACE 2 oligomer comprises an ACE2 trimer. In some embodiments, the ACE2 oligomer is an ACE2 trimer, tetramer, pentamer, hexamer and heptamer. In some embodiments, the oligomerization motif is a coil coiled motif, a foldon motif or a three helix bundle motif.

In some embodiments, the linker is a flexible linker or a rigid linker. In some embodiments, the linker is selected from the group consisting of GS(EAAAK)₅GS (SEQ ID NO: 40), AH5 (SEQ ID NO: 41), GGGH5 (SEQ ID NO: 42), H3 (SEQ ID NO: 43), H4 (SEQ ID NO: 44), H6 (SEQ ID NO: 45), H7 (SEQ ID NO: 46), AP12 (SEQ ID NO: 47), AP 15 (SEQ ID NO: 48), (GGGGS)₅ (SEQ ID NO: 49), and (EAAAK)₅ (SEQ ID NO: 50). In some embodiments, the linker has 1, 2, 3, 4 or 5 amino acid substitution as compared to SEQ ID NO: 40, 41, 42, 43, 44, 45, 46, 47, 48, 49 or 50. In some embodiments, the linker has the same length as SEQ ID NO: 40, 41, 42, 43, 44, 45, 46, 47, 48, 49 or 50. In some embodiments, the linker comprises SEQ ID NO: 40, 41, 42, 43, 44, 45, 46, 47, 48, 49 or 50, and has 1, 2, 3, 4 or 5 additional amino acids at one or both ends of SEQ ID NO: 40, 41, 42, 43, 44, 45, 46, 47, 48, 49 or 50. In some embodiments, the linker comprises (EAAAK)n, wherein n can be any integer from 3 to 15. In some embodiments, the linker comprises (AP)n, wherein n can be any integer from 8 to 22.

In some embodiments, the soluble ACE2 comprises a sequence as set forth in SEQ ID NO: 3, 51, 52, 53, 54 or 55. In some embodiments, the soluble ACE2 comprise a sequence that is at least 90%, 91%, 92%, 93%, 94%, 95%, 96%, 97%, 98%, 99% identical to SEQ ID NO: 3, 51, 52, 53, 54 or 55. In some embodiments, the soluble ACE2 has the same length as SEQ ID NO: 3, 51, 52, 53, 54 or 55. In some embodiments, the soluble ACE2 has 1, 2, 3, 4, 5, 6, 7, 8, 9, 10, 11, 12, 13, 14, 15, 16, 17, 18, 19 or 20 additional amino acids at one or both ends of SEQ ID NO: 3, 51, 52, 53, 54 or 55.

In another aspect, the present inventions provide a composition comprising the ACE2 oligomers. In some embodiments, the composition is a pharmaceutical composition and comprises a pharmaceutically acceptable carrier.

In another aspect, the present inventions provide a use of the oligomer or the composition comprising the oligomer in manufacturing a medicament for treating coronavirus infection. In another aspect, the present inventions provide a use of the oligomer or the composition comprising the oligomer in manufacturing a composition for preventing coronavirus infection. In another aspect, the present inventions provide a use of the oligomer or the composition comprising the oligomer in manufacturing a composition for detecting coronavirus in a sample. In some embodiments, the coronavirus is SARS-CoV, SARS-CoV-2 and/or SARSr-CoV. In some embodiments, the coronavirus is a mutant of SARS-CoV, a mutant of SARS-CoV-2, and/or a mutant of SARSr-CoV.

In another aspect, the present inventions provide an ACE2 oligomer as described above or a composition comprising the ACE2 oligomer for treating or preventing coronavirus infection. In another aspect, the present inventions provide an ACE2 oligomer as described above or a composition comprising the ACE2 oligomer for detecting coronavirus in a sample. In some embodiments, the coronavirus is SARS-CoV, SARS-CoV-2 and/or SARSr-CoV. In some embodiments, the coronavirus is a mutant of SARS-CoV, a mutant of SARS-CoV-2, and/or a mutant of SARSr-CoV.

In another aspect, the present inventions provide a method of treating coronavirus infection, comprising administering to a subject a therapeutically effective amount of the ACE2 oligomer or the composition as defined above. In another aspect, the present inventions provide a method of preventing coronavirus infection, comprising administering to a subject a prophylactically effective amount of the ACE2 oligomer or the composition as defined above. In another aspect, the present inventions provide a method of detecting coronavirus in a sample, comprising obtaining a sample, and contacting the sample with the ACE2 oligomer or the composition as described above. In some embodiments, the coronavirus is SARS-CoV, SARS-CoV-2 and/or SARSr-CoV. In some embodiments, the coronavirus is a mutant of SARS-Cov, a mutant of SARS-CoV-2, and/or a mutant of SARSr-CoV.

### BRIEF DESCRIPTION OF THE DRAWINGS

FIG. 1 ACE2 trimerization strategy and potential interactions between trimeric ACE2 and spike protein trimer. A. Structures of the two ACE2 trimerization motifs. B. Each ACE2 trimer can engage three RBDs either from the same spike protein (mode 1) or different spike proteins (mode 2). C. Only two ACE2s from the trimer can engage two RBDs either from the same spike protein (mode 3) or different spike proteins (mode 4).

FIG. 2 Binding affinity measurement between ACE2 proteins and SARS-CoV-2 spike protein ectodomain (S-ECD). A. Binding affinities measured using ELISA assay (n=3). B-F. Binding affinities measured using biolayer interferometry.

FIG. 3 ACE2 proteins inhibition of SARS-CoVs pseudotyped viruses (n=3). A. Inhibition of SARS-CoV-2. B. Inhibition of SARS-CoV. C-J. ACE2-rigid-foldon (T-ACE2) inhibition of SARS-CoV-2 mutants. K. ACE2-rigid-foldon (T-ACE2) inhibition of SARS-CoV similar virus WIV1. The cells used in A-K were huh-7 cells.

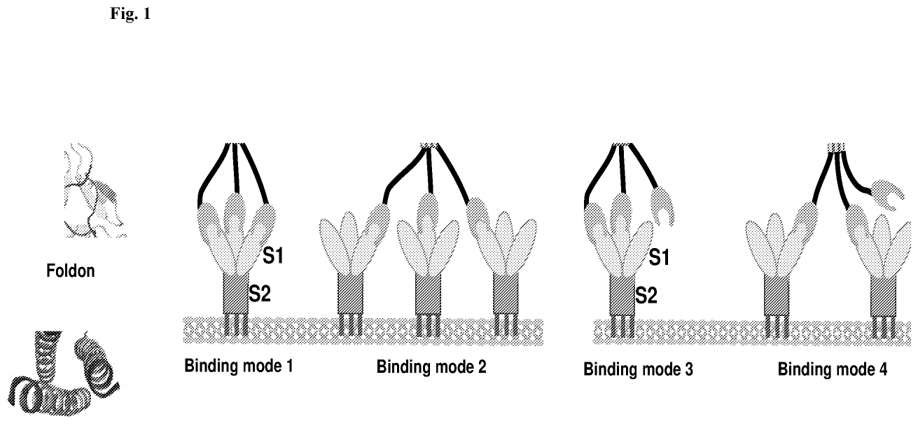

L-M. SARS-CoV-2 inhibition by ACE2-rigid-foldon (T-ACE2), ACE2-H3-foldon (H3), ACE2-GGGH5-foldon (G3H5), ACE2-AP12-foldon (AP12), ACE2-AP15-foldon (APIS), ACE2-H6-foldon (H6), ACE2-AH5-foldon (AH5), ACE2-H4-foldon (H4), ACE2-H7-foldon (H7). The cells used in L were Caco-2 cells, and the cells used in M were huh-7 cells.

N. SARS-CoV-2 inhibition by ACE2-rigid-foldon (T-ACE2), ACE2-AP15-foldon (APIS), ACE2 M1-AP15-foldon (M1), ACE2 M2-AP15-foldon (M2), ACE2 M3 -AP15-foldon (M3), ACE2 M4-AP15-foldon (M4) and ACE2 M5-AP15-foldon (M5). The cells used in N were Caco-2 cells.

O. SARS-CoV-2 inhibition by ACE2-rigid-foldon with GFP cleaved (T-ACE2-Cut), ACE2-AP15-foldon with GFP cleaved (AP15-Cut), ACE2 M1-AP15-foldon with GFP cleaved (M1-Cut), ACE2 M2-AP15-foldon with GFP cleaved (M2-Cut), ACE2 M3-AP15-foldon with GFP cleaved (M3-Cut), ACE2 M4-AP15-foldon with GFP cleaved (M4-Cut) and ACE2 M5-AP15-foldon with GFP cleaved (M5-Cut). The cells used in O were Caco-2 cells.

FIG. 4. ACE2-rigid-foldon (T-ACE2) inhibition of authentic SARS-CoV-2 virus (n=3).

FIG. 5. Cryo-EM structure of the ACE2 and S-ECD complex. The domain-colored cryo-EM map of the complex is shown on the left, and two perpendicular views of the overall structure are shown on the right. The three ACE2 are colored blue, green and violet, respectively. The RBDs of the trimeric spike protein are colored orange.

FIG. 6. Purifications and characterizations of ACE2 proteins. A-C. SDS-page gel analyses of ACE2 proteins. D. Size-exclusion chromatography analyses of ACE2 proteins.

FIG. 7. ELISA binding measurement. A. S-ECD loading amount optimization. B. Short linker ACE2 proteins binding affinities to S-ECD determined in ELISA assay (n=3).

FIG. 8. Binding affinity measurement between ACE2 proteins and SARS-CoV-2 spike protein ectodomain (S-ECD). Low loading means S-ECD was loaded at thickness signal is 0.3 nm, normal loading is 0.6 nm thickness signal.

FIG. 9. Short linker ACE2 proteins inhibition of SARS-CoV-2 pseudotyped viruses.

FIG. 10 Cryo-EM analysis of S-ECD in complex with ACE2. (A) Representative SEC purification profile of the S-ECD of SARS-CoV-2 in complex with ACE2. (B) Euler angle distribution in the final 3D reconstruction of S-ECD of SARS-CoV-2 bound with ACE2 complex. (C) Representative cryo-EM micrograph and 2D class averages of cryo-EM particle images. The scale bar in 2D class averages is 10 nm. (D) and (E) Local resolution maps for the 3D reconstruction of the RBD-ACE2 sub-complex and overall structure, respectively. (F) FSC curve of the overall structure (blue) and RBD-ACE2 sub-complex (orange). (G) FSC curve of the refined model of S-ECD of SARS-CoV-2 bound with ACE2 complex versus the overall structure that it is refined against (black); of the model refined against the first half map versus the same map (red); and of the model refined against the first half map versus the second half map (green). The small difference between the red and green curves indicates that the refinement of the atomic coordinates is not enough overfitting. (H) FSC curve of the refined model of RBD-ACE2 sub-complex, which is the same as the (G).

FIG. 11. Flowchart for cryo-EM data processing.

FIG. 12. Structural analysis and Representative cryo-EM map densities of S-ECD in complex ACE2. (A) Structural alignment in the interface of RBD and ACE2 with the RBD-PD complex previously reported (PDB ID: 6M0J) with a root mean squared deviation of 0.776 Å over 178 pairs of Cα atoms (B) Superposition in local map of RBD-ACE2 sub-complex for three protomer, which has no difference among three maps. (C) Representative cryo-EM map densities of S-ECD in complex ACE2, all densities are shown at threshold of 5 σ.

FIG. 13. Structural alignment of three protomer for S-ECD in complex ACE2. (A) Superposition in local map of RBD-ACE2 sub-complex for three protomer, which has no difference among three maps. The three ACE2 are colored blue, green and violet, respectively. (B) Structural alignment of three monomer of S-ECD in complex ACE2.

### DETAILED DESCRIPTION OF THE INVENTION

“ACE2” or “angiotensin converting enzyme II”, is a type I cell-surface glycoprotein and is found in human and mammals (such as primate, bat, cat, dog, horse, mouse, rat, hamster, pig, cattle). Unless otherwise specified, ACE2 as used herein encompasses wild-type ACE2 and all the naturally-existing variants from any human and mammal species, as well as engineered ACE2. Human ACE2 is typically composed of 805 amino acids, with amino acids 1-17 being a N-terminal signal peptide, amino acids 18-740 being extracellular, amino acids 741-761 being transmembrane, and amino acids 762-805 being cytoplasmic. ACE2 comprises a peptidase domain (PD) (residues 18-615) with its HEXXH zinc binding metalloprotease motif, a Collectrin (a regulator of renal amino acid transport and insulin)-like domain (CLD) (residues 616-768) that includes a ferredoxin-like fold “Neck” domain, that end with an hydrophobic transmembrane hydrophobic helix region of 22 amino acid residues followed by an intracellular segment of 43 amino acid residues. Many human ACE2 variants have been identified, for example, those that include any one or any combination of the following mutations: S19P, I21V, E23K, K26R, T27A, N64K, T92I, Q102P, H378R, K31R, N33I, H34R, E35K, E37K, D38V, Y50F, N51S, M62V, K68E, F72V, Y83H, G326E, G352V, D355N, Q388L or D509Y (Human ACE2 receptor polymorphisms predict SARS-CoV-2 susceptibility Stawiski et al., 2020 (https://doi.org/10.1101/2020.04.07.024752)). ACE2 is found to be expressed in lungs, arteries, heart, kidney, intestines etc. and has diverse biological functions, including regulation of blood pressure through the renin-angiotensin-aldosterone system (RAAS). ACE2 also serves as the entry point into cells for some coronaviruses, including HCoV-NL63, SARS-CoV, and SARS-CoV-2. More specifically, the binding of the spike protein of SARS-CoV and SARS-CoV-2 to the enzymatic domain of ACE2 on the surface of cells results in endocytosis and translocation of both the virus and the enzyme into endosomes located within cells.

In some embodiments, ACE2 may be a human ACE2. In some embodiments, the human ACE2 may be the ACE2 of the sequence set forth under SEQ ID NO: 1 (wild-type). In some embodiments, the human ACE2 may be any naturally-existing or engineered ACE2 mutants or variants that retain a certain level of binding affinity to a spike protein of a coronavirus as compared to SEQ ID NO: 1, for example, mutants or variants having at least 70%, 75%, 80%, 85%, 90%, 91%, 92%, 93%, 94%, 95%, 96%, 97%, 98% or 99% sequence identity to SEQ ID NO: 1, and retaining at least 70%, 75%, 80%, 85%, 90%, 91%, 92%, 93%, 94%, 95%, 96%, 97%, 98%, 99% or 100% of the binding affinity, or has increased binding affinity, to a spike protein of a coronavirus as compared to the ACE2 of SEQ ID NO: 1. In some embodiments, the ACE2 mutant may have the length of SEQ ID NO: 1 and have 1, 2, 3, 4, 5, 6, 7, 8, 9, 10, 11, 12, 13, 14, 15, 16, 17, 18, 19, or 20 amino acid substitutions as compared to SEQ ID NO: 1. In some embodiments, the ACE2 mutant may have the same length as SEQ ID NO: 1 and have K31F, H34I, and E35Q substitutions as compared to SEQ ID NO: 1 (Proc Natl Acad Sci U S A. 2020 Nov 10;117(45):28046-28055. Wells JA.). In some embodiments, the ACE2 mutant may have the same length as SEQ ID NO: 1 and have T27Y, L79Y, and N330Y substitutions as compared to SEQ ID NO: 1 (Science. 2020 Sep4;369(6508):1261-1265. Procko E.). In some embodiments, the ACE2 mutant may have the same length as SEQ ID NO: 1 and have T27Y, and H34A substitutions as compared to SEQ ID NO: 1 (Sci Rep 11, 12740 (2021) Tanaka, S.). In some embodiments, the ACE2 mutant may have the same length as SEQ ID NO: 1 and have T27Y, K31F, H34I, E35Q, L79Y, and N330Y substitutions as compared to SEQ ID NO: 1. In some embodiments, the ACE2 mutant may have the same length as SEQ ID NO: 1 and have T27Y, H34A, L79Y, and N330Y substitutions as compared to SEQ ID NO: 1.

“Soluble ACE2” as used herein refers to an ACE2 as described above but lacks the transmembrane and cytoplasmic residues. In some embodiments, soluble ACE2 may comprise the entire extracellular domain. In some embodiments, soluble ACE2 may comprise a part of the extracellular domain. In some embodiments, soluble ACE2 may be the ACE2 that is composed of residues 1-740 of SEQ ID NO: 1 (i.e., SEQ ID NO: 2), or may be any functional fragments thereof. Functional fragments mean any fragments that retain at least 70%, 75%, 80%, 85%, 90%, 91%, 92%, 93%, 94%, 95%, 96%, 97%, 98%, 99% or 100% of the binding affinity, or has increased binding affinity, to a spike protein of a coronavirus as compared to SEQ ID NO: 3 (SEQ ID NO: 3 denotes amino acids 18-615 of SEQ ID NO: 1). Soluble ACE2 also encompasses variants or mutants of the above functional fragments that have at least 70%, 75%, 80%, 85%, 90%, 91%, 92%, 93%, 94%, 95%, 96%, 97%, 98% or 99% sequence identity to the above functional fragments, and retaining at least 70%, 75%, 80%, 85%, 90%, 91%, 92%, 93%, 94%, 95%, 96%, 97%, 98%, 99% or 100% of the binding affinity, or has increased binding affinity, to a spike protein of a coronavirus as compared to the ACE2 of SEQ ID NO: 3. In some embodiments, the soluble ACE2 may be SEQ ID NO: 3. In some embodiments, the soluble ACE2 may be of at least 70%, 75%, 80%, 85%, 90%, 91%, 92%, 93%, 94%, 95%, 96%, 97%, 98% or 99% sequence identity to SEQ ID NO: 3. In some embodiments, the soluble ACE2 may have the length of SEQ ID NO: 3 and have 1, 2, 3, 4, 5, 6, 7, 8, 9, 10, 11, 12, 13, 14, 15, 16, 17, 18, 19, or 20 amino acid substitution as compared to SEQ ID NO: 3. In some embodiments, the soluble ACE2 may be M1 (SEQ ID NO: 51), which has residues 18-615 of SEQ ID NO: 1 and has K31F, H34I, and E35Q substitutions as compared to SEQ ID NO: 1. In some embodiments, the soluble ACE2 may be M2 (SEQ ID NO: 52), which has residues 18-615 of SEQ ID NO: 1 and hasT27Y, L79Y, and N330Y substitutions as compared to SEQ ID NO: 1. In some embodiments, the soluble ACE2 may be M3 (SEQ ID NO: 53), which has residues 18-615 of SEQ ID NO: 1 and has T27Y, and H34A substitutions as compared to SEQ ID NO: 1. In some embodiments, the soluble ACE2 may be M4 (SEQ ID NO: 54), which has residues 18-615 of SEQ ID NO: 1 and has T27Y, K31F, H34I, E35Q, L79Y, and N330Y substitutions as compared to SEQ ID NO: 1. In some embodiments, the soluble ACE2 may be M5 (SEQ ID NO: 55), which has residues 18-615 of SEQ ID NO: 1 and has T27Y, H34A, L79Y, and N330Y substitutions as compared to SEQ ID NO: 1.

“Coronaviruses” are a group of related RNA viruses that are roughly spherical particles with bulbous surface projections and cause diseases in mammals and birds. Coronaviruses identified thus fir include SARS-Cod' i 2003. HCoV NL63 in 2004, HICoV HKU1 in 2005, MERS-CoV in 2012, and SARS-CoV-2 in 2019. In some embodiments, the coronaviruses comprise SARS-CoV and mutants (or variants) thereof, SARS-CoV-2 and mutants (or variants) thereof, and SARS-related coronaviruses (SARSr-CoV) and mutants (or variants) thereof SARSr-CoV refers to any coronavirus strain that enters a host cell through ACE2. In some embodiments, mutants or variants of SARS-CoV, SARS-CoV-2 or SARSr-CoV refer to coronavirus strains having a. genome that is of at least 90%, 91%, 92%, 93%, 94%, 95%, 96%, 97%, 98%, 99%, 99.1%, 99..2%, 99.3%, 99.4%, 99.5%, 99.6%, 99,7%, 99.8% or 99.9% sequence identity to the genome of SARS-CoV, SARS-CoV-2 or SARSr-CoV. In some embodiments, mutants or variants of SARS-CoV, SARS-CoV-2 or SARSr-CoV exhibit different or substantially the same activities and properties as SARS-COV, SARS-CoV-2 or SARSr-CoV, In some embodiments, SARS-CoV-2 may refer to the strain of the first reported genome (SARS-CoV-2 Wuhan-Hu-1). In some embodiments, mutants or variants of SARS-CoV-2 have a mutation in the spike protein. in some embodiments, mutations in the spike protein include a substitution selected from the group consisting of V341I, A344S, F342L, V367F, R408I, A435S, N439K, G476S, V483A, and D614G, as compared to the sequence of the spike protein of SAR S-CoV-2 Wuhan-Hu-1.

“ACE2 monomer” refers to a monomeric peptide or protein that comprises a soluble ACE2, a linker, and an oligomerization domain. In some embodiments, the ACE2 monomer comprises from N-terminal to C-terminal a soluble ACE2, a linker and an oligomerization domain. In some embodiments, the soluble ACE2 is directly connected to the linker through a peptide bond. In some embodiments, the linker is directly , connected to the oligomerization domain through a peptide bond. Different ACE2 monomers may have different soluble ACE2, different linker and/or different oligotnerization domain. In some embodiments, the ACE2 monomer may further comprise a label for being used in coronavirus detection. In some embodiments, the label may be a fluorescence label. In some embodiments, the label may be a fluorescence protein. In some embodiments, the label may be a quantum dot.

“ACE2 oligomer” refers to oligomers formed from association of ACE2 monomers through the oligomerization domain, the association may be covalent bond and/or non-covalent interactions (e.g., electrostatic interactions (e.g., ionic, hydrogen bonding, halogen bonding), van der Waals forces (e.g., dipole-dipole, dipole-induced dipole, London dispersion forces), n-effects, hydrophobic effect) In some embodiments, the ACE2 oligomer may comprise an ACE2 timer. In some embodiments, the ACE2 oligomer may be an ACE2 heptamer, hexamer, pentamer, tetramer or trimer. In some embodiments, the ACE2 oligomer may be formed from association of identical ACE2 monomers. In some embodiments, the ACE oligomer may be formed from association of different ACE2 monomers. In some embodiments, the ACE2 oligomer may be formed through spontaneous association of the oligomerization domain of the ACE2 monomers.

“Oligomerization motif” or “oligomerization domain” refers to a motif or domain that interacts with one another and brings the monomer into association. Different oligomerization motifs or domains are known in the art. For example, naturally-existing or de novo designed coiled coil motifs that allow 2-7 alpha-helices being coiled together, to form, for example, helical bundles or helical barrels (Robust De Novo-Designed Homotetrameric Coiled Coils. Biochemistry, Edgell et al., 2020 (https://doi.org/10.1021/acs.biochem.0c00082); A Basis Set of de Novo Coiled-Coil Peptide Oligomers for Rational Protein Design and Synthetic Biology, Fletcher et al., 2012 (https://doi.org/10.1021/sb300028q); Navigating the Structural Landscape of De Novo α-Helical Bundles, Rhys et al., 2019 (https://doi.org/10.1021/jacs.8b13354); Computational design of water-soluble alpha-helical barrel, Thomson et al., 2014 (DOI: 10.1126/science.1257452); Maintaining and breaking symmetry in homomeric coiled-coil assemblies, Rhys et al., 2018 (https://doi.org/10.1038/s41467-018-06391-y). In some embodiments, the oligomerization motif may be a heptamerization motif, a hexamerization motif, a pentamerization motif, a tetramerization motif or a trimerization motif. In some embodiments, the oligomerization motif may be a coiled coil motif, a foldon motif or a three helix bundle motif.

“Linker” as used herein refers to a region that links two protein domain (e.g., a soluble ACE2 and an oligomerization motif) together. Linkers used in fusion protein technology are typically categorized into “flexible linker”, “rigid linker” and “in vivo cleavable linker”, and the standards for such categorization are well-known in the art (Fusion Protein Linkers: Property, Design and Functionality, Chen et al., 2012 (10.1016/j.addr.2012.09.039)). Flexible linkers confer flexibility in the structure, and examples of flexible linkers known in the art include (GGGGS)₃, (Gly)₈, (Gly)₆, GGGGS, (GGGGS)n (n=1, 2, 4). Flexible linker as provided in the present disclosure includes (GGGGS)₅ (SEQ ID NO: 50). Rigid linkers confer rigidity in the structure, and examples of rigid linkers known in the art include (EAAAK)n (n=1-3), A(EAAAK)₄ALEA(EAAAK)₄A, AEAAAKEAAAKA, PAPAP, (Ala-Pro)n (10-34 aa). Rigid linkers as provided in the present disclosure include GS(EAAAK)₅GS (SEQ ID NO: 40), AH5 (SEQ ID NO: 41), GGGH5 (SEQ ID NO: 42), H3 (SEQ ID NO: 43), H4 (SEQ ID NO: 44), H6 (SEQ ID NO: 45), H7 (SEQ ID NO: 46), AP12 (SEQ ID NO: 47), AP15 (SEQ ID NO: 48), and (EAAAK)₅ (SEQ ID NO: 51)).

“Treating a coronavirus infection” means reducing the amount of coronavirus or completely eliminating the presence of coronavirus in a subject, and/or alleviating one or more symptoms associated with coronavirus infection or completely eliminating the symptoms in a subject, as compared to the results in the absence of the treatment.

“Preventing a coronavirus infection” means preventing the infection of coronavirus in a subject, as compared to the results in the absence of the treatment.

“Detecting coronavirus” means detecting the presence, level or amount of coronavirus, and/or the activity of coronavirus in a sample. In some embodiments, the sample is a “biological sample” that may include body fluids (such as sputum, semen, lymph, sera, plasma, urine, synovial fluid and cerebro-spinal fluid), cell samples or tissue samples obtained from human and animals (mammals, poultry, livestock, birds etc.). In some embodiments, the samples may be an “environmental sample” or “non- biological sample” including feces, surgical fluids, water (drinking water, sea water, river water etc.), soil, food (meat, seafood, vegetables, fruits, diary etc.) and any other samples obtained from the environment. Methods of pretreating the sample such that it is suitable for detection are known in the art.

Any methods that involve using specific and/or high-affinity interactions between two protein molecules for detecting a target (such as a protein or a virus expressing a target protein) can be readily applied to the detection method of the present inventions, wherein the sensor is ACE2 oligomer and the target is coronavirus, and the specific and high-affinity interaction is between the ACE2 oligomer and the spike protein of the virus. Such detection methods are already known in the art. For examples, the detection method may involve competitive chromatography and involve attaching a florescent label such as fluorescence proteins or quantum dots (CN111273016A) to the ACE2 oligomer.

The term “pharmaceutically acceptable carrier” encompasses any of the standard pharmaceutical carriers, buffers and excipients, including buffered saline solution, water, and emulsions (such as an oil/water or water/oil emulsion), and various types of wetting agents and/or adjuvants. Suitable pharmaceutical carriers and their formulations are described in Remington's Pharmaceutical Sciences (Mack Publishing Co., Easton, 19th ed. 1995). Preferred pharmaceutical carriers depend upon the intended mode of administration of the active ingredient agent.

A “therapeutically effective amount” refers to an amount effective, at dosages and for periods of time necessary, to achieve the desired therapeutic result. A therapeutically effective amount may vary according to factors such as the state of infection, disease or disorder; age; sex; and weight of the individual. A therapeutically effective amount is also one in which any toxic or detrimental effects of the pharmaceutical composition is outweighed by the therapeutically beneficial effects.

A “prophylactically effective amount” refers to an amount effective, at dosages and for periods of time necessary, to achieve the desired prophylactic result. A prophylactically effective amount may vary according to factors such as the state of infection, disease or disorder; age; sex; and weight of the individual. A prophylactically effective amount is also one in which any toxic or detrimental effects of the pharmaceutical composition is outweighed by the prophylactically beneficial effects.

As used herein, “sequence identity”, “% sequence identity”, or “% identical” means the percentage of identical nucleotide or amino acid residues at corresponding positions in two or more sequences when the sequences are aligned to maximize sequence matching, i.e., taking into account gaps and insertions. Sequences are generally aligned for maximum correspondence over a designated region, e.g., a region at least about 20, 25, 30, 35, 40, 45, 50, 55, 60, 65 or more amino acids or nucleotides in length, and can be up to the full-length of the reference amino acid or nucleotide. For sequence comparison, typically one sequence acts as a reference sequence, to which test sequences are compared. When using a sequence comparison algorithm, test and reference sequences are input into a computer program, subsequence coordinates are designated, if necessary, and sequence algorithm program parameters are designated. The sequence comparison algorithm then calculates the percent sequence identity for the test sequence(s) relative to the reference sequence, based on the designated program parameters. Examples of algorithms that are suitable for determining percent sequence identity are the BLAST and BLAST 2.0 algorithms, which are described in Altschul et al. (1990) J. Mol. Biol. 215: 403-410 and Altschul et al. (1977) Nucleic Acids Res. 25: 3389-3402, respectively. Software for performing BLAST analyses is publicly available through the National Center for Biotechnology Information (www.ncbi.nlm.nih.gov). Further exemplary algorithms include ClustalW (Higgins D., et al. (1994) Nucleic Acids Res 22: 4673-4680), available at www.ebi.ac.uk/Tools/clustalw/index.html. The percent sequence identity between two amino acid sequences can also be determined using the algorithm of E. Meyers and W. Miller, Comput. Appl. Biosci. 4:11-17, (1988) which has been incorporated into the ALIGN program (version 2.0), using a PAM120 weight residue table, a gap length penalty of 12 and a gap penalty of 4. In addition, the percent sequence identity between two amino acid sequences can be determined using the Needleman and Wunsch, J. Mol. Biol. 48:444-453, (1970) algorithm which has been incorporated into the GAP program in the GCG software package (available at www.gcg.com), using either a Blossom 62 matrix or a PAM250 matrix, and a gap weight of 16, 14, 12, 10, 8, 6, or 4 and a length weight of 1, 2, 3, 4, 5, or 6.

The terms “a”, “an”, and “the” as used herein are intended to mean “one and more than one” or “at least one”, unless the context clearly suggests a singular meaning.

The term “and/or” as used herein are intended to include any and all possible combinations of one or more of the listed items.

The terms “comprises” and “comprising” as used herein are intended to indicate the presence of an element, component, feature, step etc., but not to exclude the presence of any other elements, components, features, steps etc. In the present invention, when it is mentioned that a product comprises certain components, a substance comprises a structure, a method comprises a step etc., it should be understood that it also recites the product which is composed only of these components, a substance that is composed only of the structure, a method that is composed only of the step etc.

### EXAMPLES

To develop trimeric ACE2 decoy proteins, we chose a C-terminal domain of T4 fibritin (foldon) (31, 32) or a three helix bundle (3HB) (33, 34) as trimerization motifs since these have been successfully demonstrated to form stable protein trimers (27, 31, 32). We then looked at the reported SARS-CoVs spike protein structures to determine the linker between trimerization motifs and ACE2 (27, 35-39). SARS-CoV spike protein mostly adopt one or two RBDs up conformations and can engage one or two ACE2 monomers (35, 36). A very small population of SARS-CoV spike protein can have three RBDs up conformation to bind three ACE2 monomers. SARS-CoV-2 structures mostly have closed conformation or one RBD up conformation (27, 37, 38). From these structural analyses, we estimated distances between RBDs on the same spike protein could range from 60 Å to 100 Å when they are in the up conformations. Moreover, structures from SARS-CoV viral particle revealed there are about 100 spike protein trimers displayed on the 100 nm diameter viral particle surface giving inter spike protein distance around 200 Å (3, 40, 41).

To retain the possibility for intra-spike or inter-spike avidity, we chose a flexible (GGGGS)₅ linker or a more rigid linker selected from the group consisting of GS(EAAAK)₅GS linker (SEQ ID NO: 40), AH5 linker (SEQ ID NO: 41), GGGHS linker (SEQ ID NO: 42), H3 linker (SEQ ID NO: 43), H4 linker (SEQ ID NO: 44), H6 linker (SEQ ID NO: 45), H7 linker (SEQ ID NO: 46), AP12 linker (SEQ ID NO: 47), and APIS linker (SEQ ID NO: 48) to construct trimeric ACE2 (42). We used ACE2 peptidase domain (18-615) (SEQ ID NO: 3, 51, 52, 53, 54 or 55) to construct all trimeric ACE2 decoy proteins, linkers were inserted after ACE2, followed by the trimerization motifs. We therefore constructed seventeen ACE2 monomers, for forming trimeric ACE2 proteins. The structure of the monomers are: ACE2-flexible-3HB (SEQ ID NO: 11) (flexible denotes the (GGGGS) s linker), ACE2-rigid-3HB (SEQ ID NO: 7) (rigid denotes the GS(EAAAK)5GS linker), ACE2-flexible-foldon (SEQ ID NO: 9), ACE2-rigid-foldon (SEQ ID NO: 5), ACE2-AH5-foldon (SEQ ID NO: 15), ACE2-GGGHS-foldon (SEQ ID NO: 17), ACE2-H3-foldon (SEQ ID NO: 19), ACE2-H4-foldon (SEQ ID NO: 21), ACE2-H6-foldon (SEQ ID NO: 23), ACE2-H7-foldon (SEQ ID NO: 25), ACE2-AP12-foldon (SEQ ID NO: 27), ACE2-AP15-foldon (SEQ ID NO: 29) , ACE2 M1-AP15-foldon (SEQ ID NO: 31), ACE2 M2-AP15-foldon (SEQ ID NO: 33), ACE2 M3-AP15-foldon (SEQ ID NO: 35), ACE2 M4-AP15-foldon (SEQ ID NO: 37), and ACE2 M5-AP15-foldon (SEQ ID NO: 39) (the structure is shown from N terminus to C terminus). In addition, we constructed two trimeric ACE2 proteins with a short linker GGGS (ACE2-short-3HB, ACE2-short-foldon) and a monomeric ACE2 as control proteins.

### Results

Note that all the ACE2 monomers were first constructed and obtained with an HRV3C cleavage sequence, an eGFP tag and a His8 tag (together termed the C-terminal tag) following the trimerization domain. Unless specified otherwise, the ACE2 monomer or trimer used below comprise the C-terminal tag.

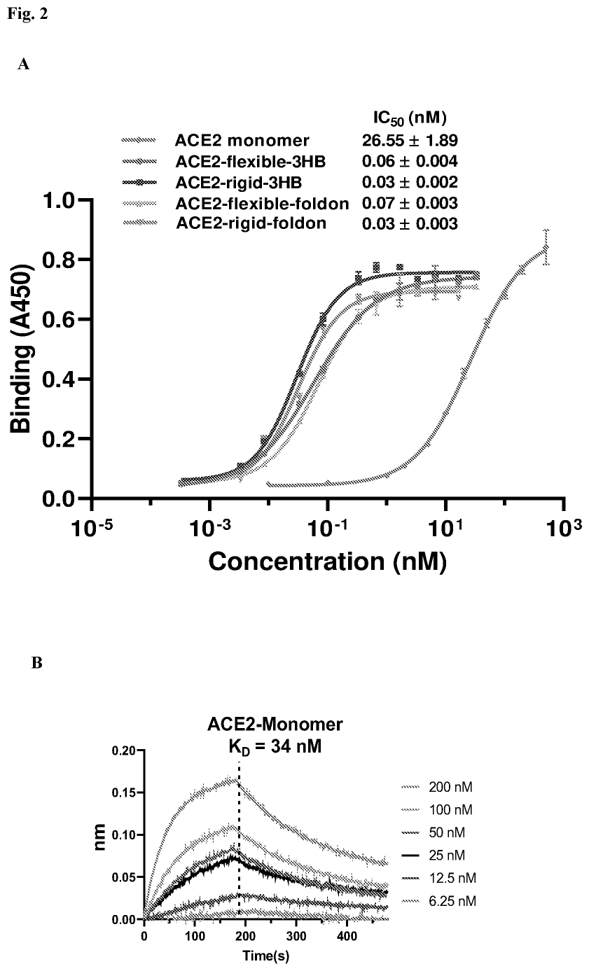

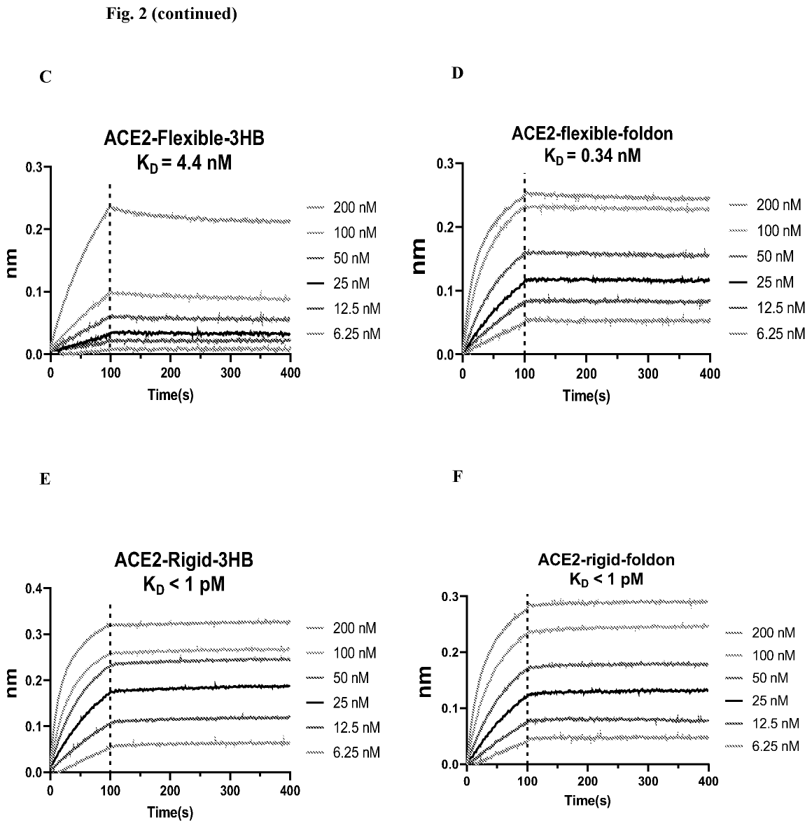

We first used ELISA assay to determine binding affinities between ACE2 proteins and the prefusion stabilized trimeric SARS-CoV-2 spike protein ectodomain (S-ECD) (27). ACE2 monomer binds S-ECD with IC₅₀˜27 nM. For trimeric ACE2 proteins, we saw massive binding affinity enhancement. Rigid linker constructs have highest binding affinities, ACE2-rigid-3HB and ACE2-rigid-foldon both bind S-ECD with IC₅₀˜30 pM (FIG. 2A). Trimeric ACE2 proteins with short linkers have lower binding affinities (FIG. 7).

We further analyzed ACE2 proteins binding using biolayer interferometry (ForteBio Octet RED96) (FIG. 2B-F). S-ECD was biotinylated with NHS-PEG8-Biotin and was loaded on streptavidin coated senor at about 25% saturation to avoid artificial inter-molecular avidity. Different concentrations of ACE2 proteins were then used as analytes to measure binding affinities. K_{D} for ACE2 monomer/S-ECD is 34 nM and this agrees well with previously published results (2 7) . For trimeric ACE2 proteins, we also saw dramatic increased binding affinities. K_{D} for ACE2-flexible-3HB/S-ECD is 4.4 nM while K_{D} for ACE2-flexible-foldon/S-ECD goes down to 0.34 nM. Both ACE2-rigid-3HB and ACE2-rigid-foldon bind S-ECD extremely well (K_{D}<1 pM). Further decreasing loading of S-ECD on streptavidin sensor didn't affect ACE2 proteins binding and this suggests intra-molecular avidity binding between trimeric ACE2s and S-ECD (FIG. 8). The massive binding affinity enhancement for ACE2-rigid-3HB and ACE2-rigid-foldon also indicates spike protein probably has at least two RBDs in the up conformation upon binding. Short linker ACE2 proteins binds better than ACE2 monomer, but not as good as rigid linker ACE2 proteins (FIG. 8).

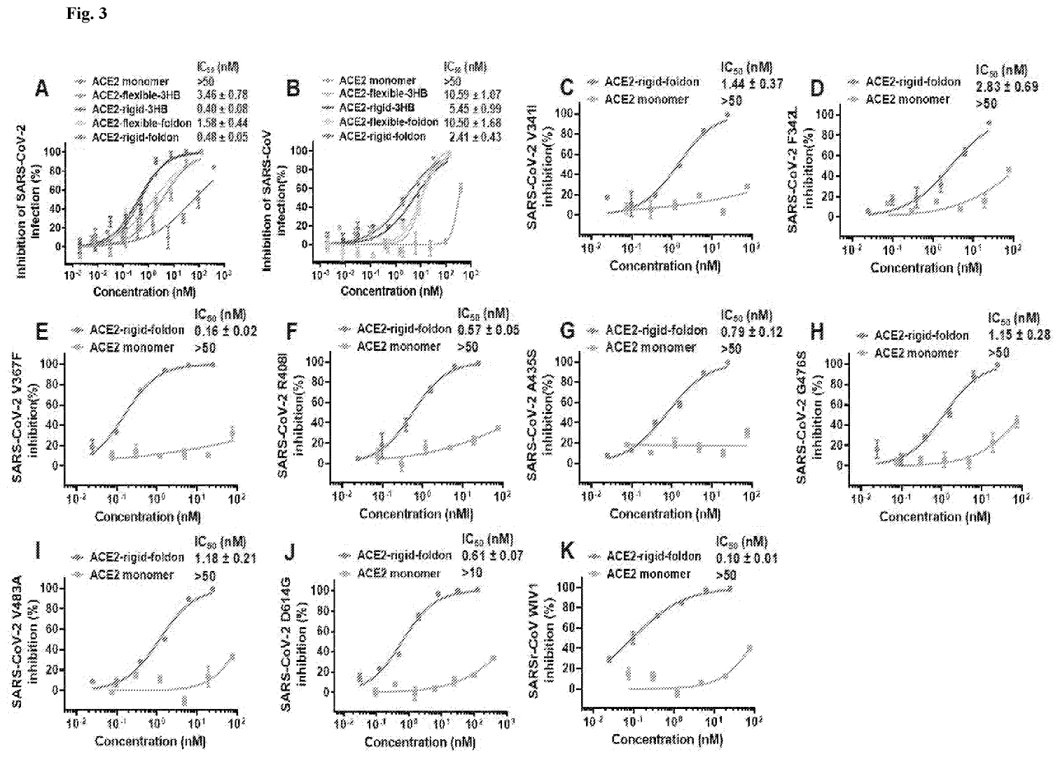

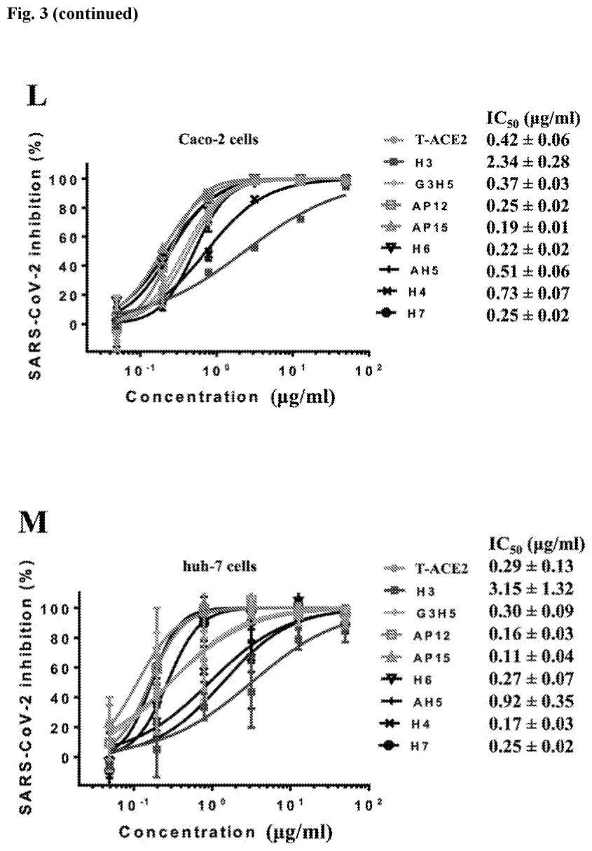

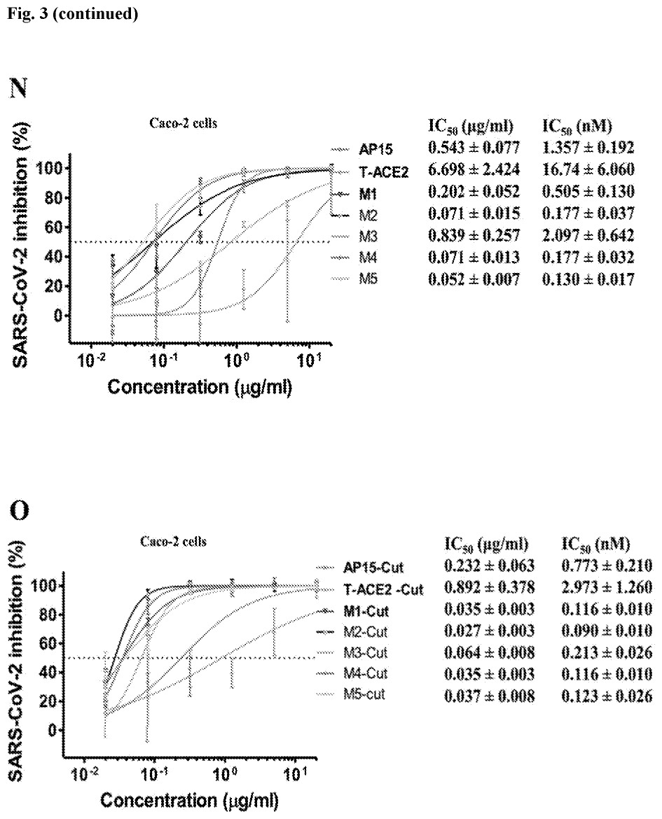

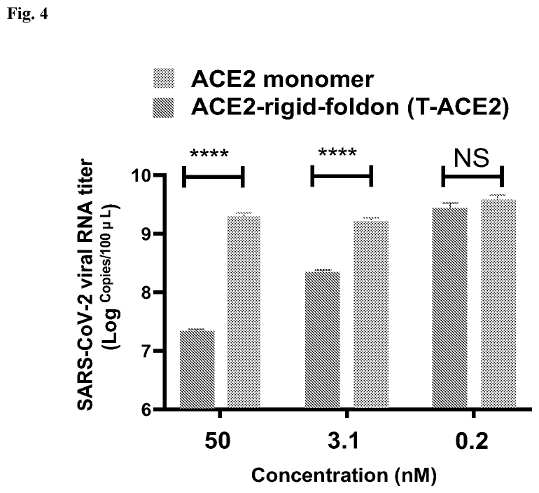

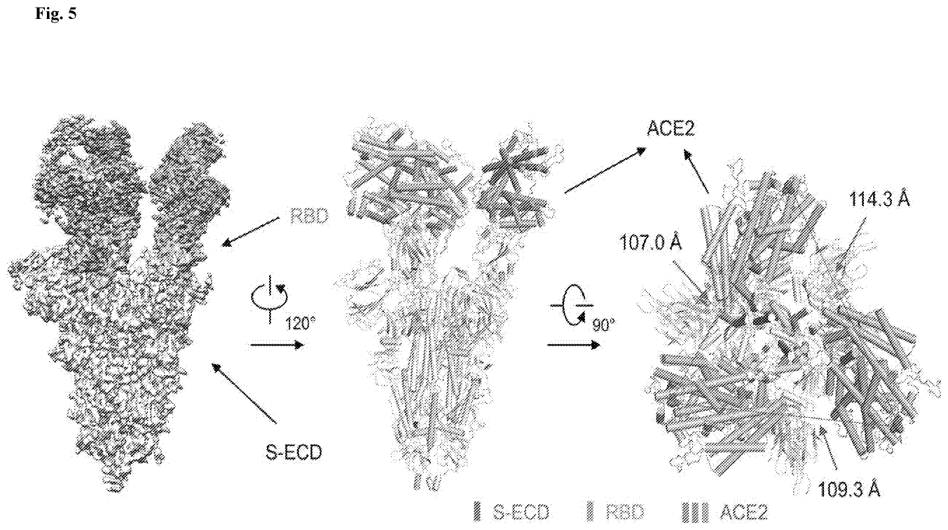

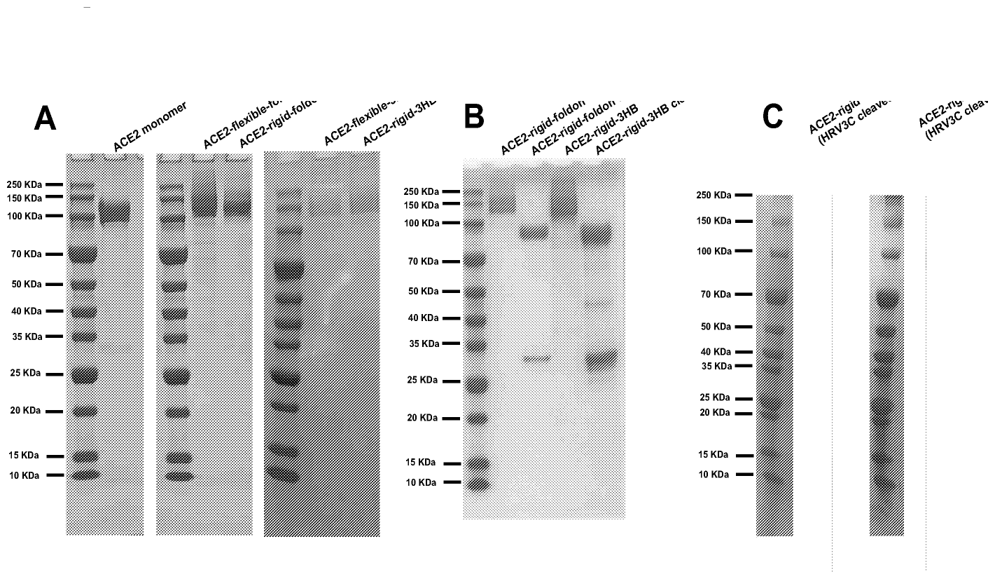

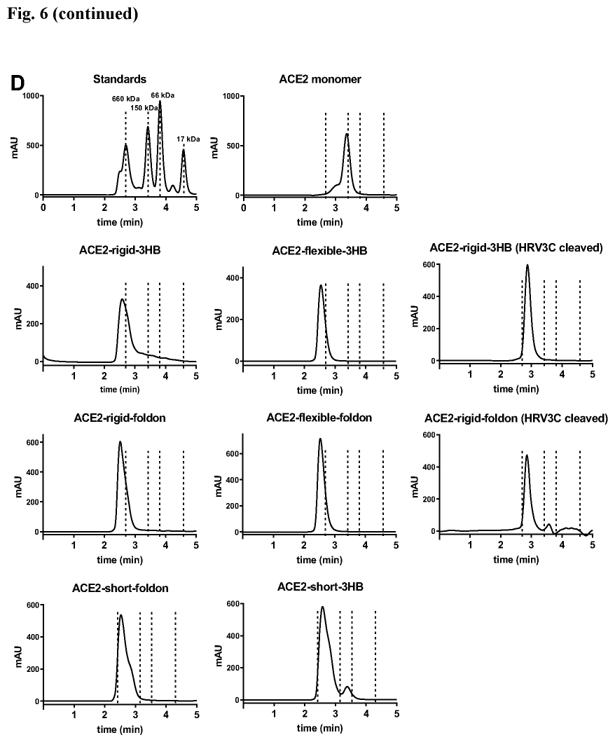

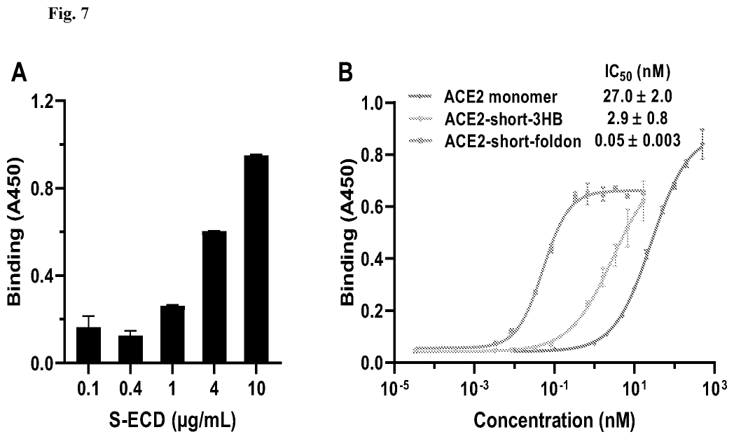

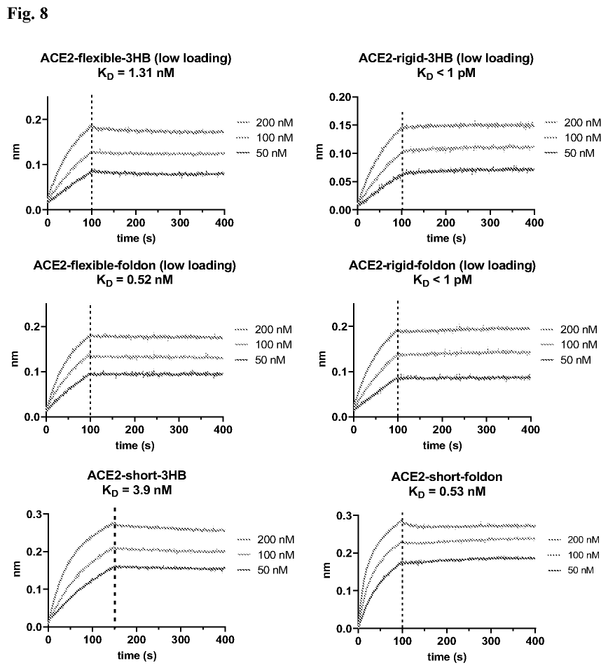

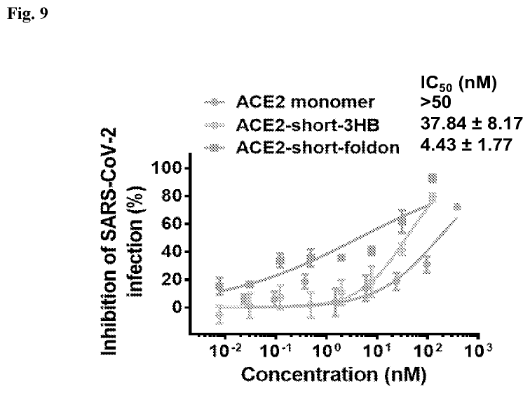

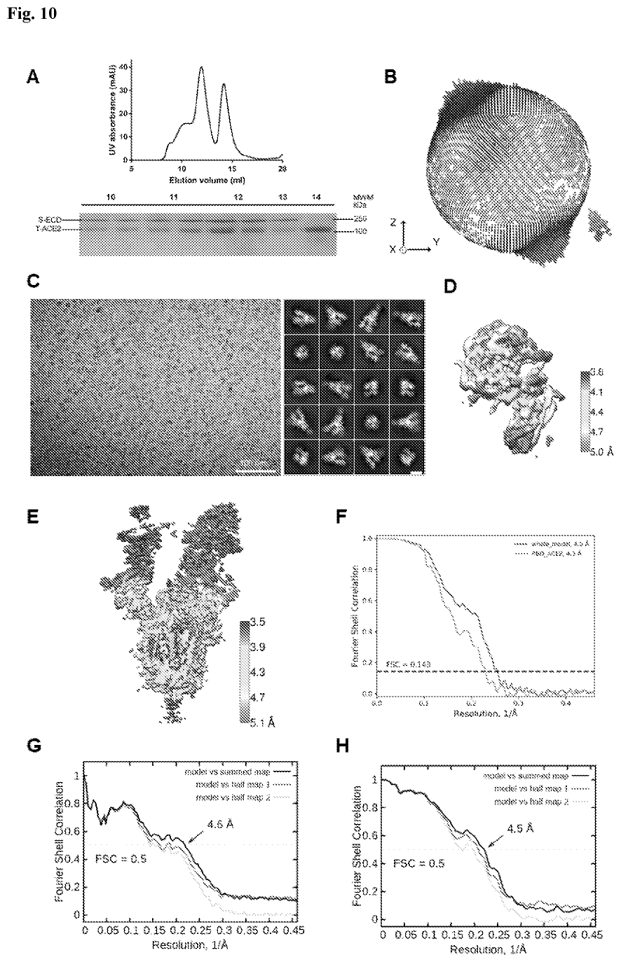

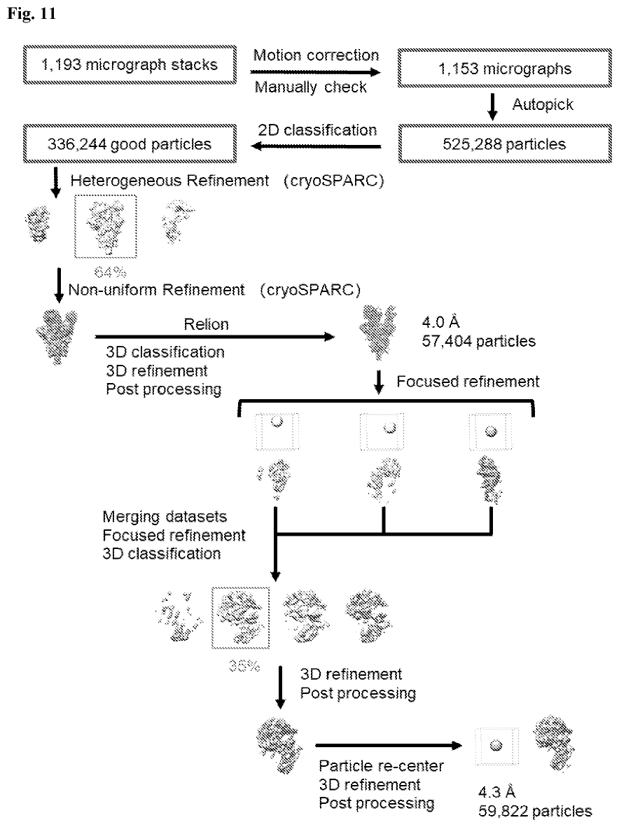

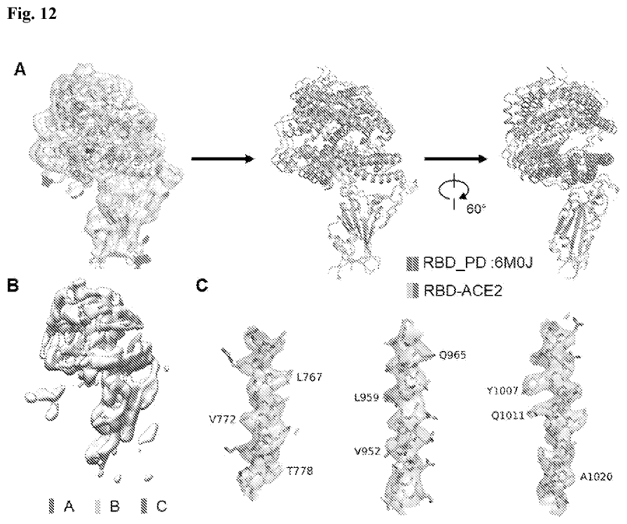

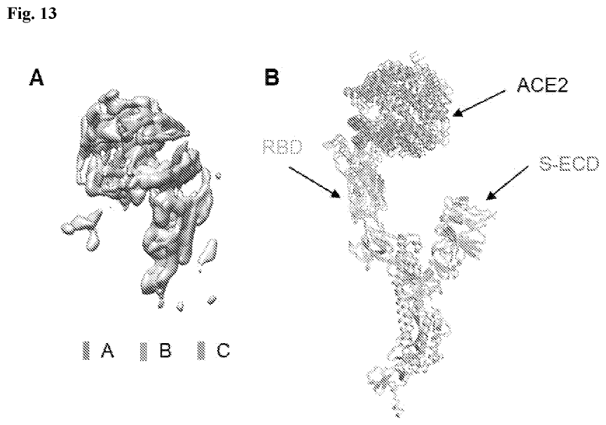

Next, we assessed the inhibitory activities of these trimeric ACE2 decoy proteins using SARS-CoV-2 and SARS-CoV pseudotyped viruses. ACE2 monomer can only inhibit SARS-CoV-2 pseudotyped virus at high concentration with IC₅₀>50 nM. Trimeric ACE2 with flexible linkers shown much better inhibition activity, ACE2-flexible-3HB can inhibit SARS-CoV-2 with IC₅₀ of 3.46 nM, ACE2-flexible-foldon has better inhibition activity with IC₅₀ of 1.58 nM (FIG. 3A). Rigid linker trimeric ACE2 proteins have best inhibition activities: ACE2-rigid-3HB has IC so of 0.40 nM, ACE2-rigid-foldon has IC₅₀ of 0.48 nM (FIG. 3A). Short linker trimeric ACE2 proteins didn't show apparent improved inhibition activities comparing with ACE2 monomer even though they have higher binding affinities than ACE2 monomer (FIG. 9). For SARS-CoV pseudotyped virus inhibition, we saw similar results. ACE2 monomer has weak inhibition activity with IC₅₀>50 nM, ACE2-rigid-foldon has best inhibition activity with IC₅₀ of 2.41 nM (FIG. 3B), we therefore designate ACE2-rigid-foldon as T-ACE2.

Because of the significantly advantageous effect attained by T-ACE2, we designed more trimeric ACE2 using different rigid linkers and compared these trimeric ACE2 with T-ACE2. Specifically, we made ACE2-H3-foldon (H3), ACE2-GGGHS-foldon (G3H5), ACE2-AP12-foldon (AP 12), ACE2-AP 1 5 -fol don (APIS), ACE2-H6-fol don (H6), ACE2-AH5-foldon (AH5), ACE2-H4-foldon (H4), and ACE2-H7-foldon (H7). FIGS. 3L-M shows the IC₅₀ for inhibiting SARS-CoV-2 pseudotyped virus of the aforementioned trimeric ACE2. It can be seen that G3H5, AP12, APIS, H6 and H7 all attained an IC₅₀ even lower than that of T-ACE2 in inhibiting SARS-CoV-2 pseudotyped virus in Caco-2 cells, and AH5 and H4 both attained an IC₅₀ of a similar level to that of T-ACE2 (FIG. 3L). In addition, AP12, APIS, H6, H4 and H7 all attained an IC so even lower than that of T-ACE2 in inhibiting SARS-CoV-2 pseudotyped virus in huh-7 cells, and G3H5 and AH5 both attained an IC so of a similar level to that of T-ACE2 (FIG. 3M).

We also designed more trimeric ACE2 using the APIS linker and different soluble ACE2 mutants. Specifically, we made ACE2 M1-AP15-foldon (M1), ACE2 M2-AP15-foldon (M2), ACE2 M3-AP15-foldon (M3), ACE2 M4-AP15-foldon (M4), and ACE2 M5-AP15-foldon (M5). We also made T-ACE2 with the C-terminal tag cleaved (T- ACE2-Cut), AP 15 with the C-terminal tag cleaved (AP15-Cut), M1 with the C-terminal tag cleaved (M1-Cut), M2 with the C-terminal tag cleaved (M2-Cut), M3 with the C-terminal cleaved (M3-Cut), M4 with the C-terminal tag cleaved (M4-Cut), and M5 with the C-terminal tag cleaved (MS-Cut). FIGS. 3N-O shows the IC₅₀ for inhibiting SARS-CoV-2 pseudotyped virus of the aforementioned trimeric ACE2. It can be seen that APIS, M1, M2, M3, M4, M5, AP15-Cut, M1-Cut, M2-Cut, M3-Cut, M4-Cut and M5-Cut all attained significantly lower IC₅₀ compared to T-ACE2-Cut in inhibiting SARS-CoV-2 pseudotyped virus in Caco-2 cells.

We then asked whether T-ACE2 can also inhibit SARS-CoV-2 mutants and related coronaviruses. We tested T-ACE2 inhibition activities on eight naturally occurring SARS-CoV-2 mutants including seven RBD domain mutations (14, 16), and D614G mutation (43); and the SARSr-CoV (WIV1) (FIGS. 3C-K). We gladly found T-ACE2 can potently inhibit all these viruses. We believe novel mutations identified in the future are not likely to escape ACE2, the impressive performance of T-ACE2 prompt us to believe T-ACE2 will have high probability to inhibit most of those novel mutants if not all.

We further tested T-ACE2 inhibition of authentic SARS-CoV-2 virus (FIG. 4). Importantly, we found T-ACE2 can also potently inhibit authentic SARS-CoV-2, which agrees well with our binding affinity and pseudotyped virus inhibition results. In addition, we found that AH5, GGGH5, H3, H4, H6, H7, AP12, AP15, M1, M2, M3, M4, M5, AP15-Cut, M1-Cut, M2-Cut, M3-Cut, M4-Cut and M5-Cut can also potently inhibit authentic SARS-CoV-2.

We hypothesized properly designed trimeric ACE2 might engage more than one RBD from trimeric spike protein and thus dramatically increase binding affinity through avidity effect. To confirm this unique engagement, we determined the complex structure of T-ACE2/S-ECD using cryo-EM. In the complex structure, spike protein adopts only one conformation: the three RBDs up conformation. The complex is near perfect three-fold symmetric. Significantly, all these three RBDs bind to three ACE2s simultaneously, binding interactions between ACE2 and RBD are essentially the same as previous reports and the three individual monomer from the complex aligns quite well (FIG. 13) (44, 45). Although we couldn't observe the linker and trimerization motif, we are confident that the three ACE2s binding to the same spike protein are from the same trimer because of the binding affinity data and virus inhibition data. This spike protein conformation is very different than previously reported prefusion stabilized spike protein structure where only one or none RBD is in the up conformation. Recent complex structures between SARS-CoV-2 spike protein and ACE2 monomer from preprints indicate monomer ACE2 binding can indeed induce conformational changes of spike protein, and some spike protein can have two or three RBDs in the up conformations to bind up to three ACE2s (46, 47). Here, in our structure, the unique three RBDs up conformation in all the spike proteins should indeed be induced by our trimeric ACE2.

The distance between the C-terminal end of the three ACE2s is around 110 Å. If the trimerization motif sits right in the middle, then ideal linker length between trimerization motif and ACE2 would be around 60 Å, corresponding to (GGGGS)₃ linker, thus the (GGGGS)₅ flexible linker in our proteins is long enough for three ACE2 binding but is not optimal. The more rigid (EAAAK) 5 linker is shorter than (GGGGS)₅ and can effectively separate different functional domains of fusion proteins (48). We think the (EAAAK)₅ linker length is probably around 60 Å making it an optimal linker for T-ACE2, the rigidity nature of this (EAAAK) 5 linker probably helps to keep ACE2 right around RBD for immediate rebinding even if one of the ACE2 falls off spike protein. This probably explains the strong trimer-trimer avidity binding.

### Discussion

Since the beginning of COVID-19, tremendous efforts have been made to develop therapeutics especially neutralizing antibodies to treat COVID-19. However, the widespread and ongoing crisis of COVID-19 indicates SARS-CoV-2 will not be eliminated soon, thus unexpected mutations making current neutralizing antibodies ineffective could develop in the future. Furthermore, the emergence of COVID-19 after SARS suggests similar coronavirus pandemic might happen in the future. These calls for therapeutic approaches widely useful for current and future similar coronaviruses and mutants.

Several engineered ACE2 proteins bearing different number of mutations have been shown to increase spike protein binding affinities and virus neutralization activities (29, 49). Here, we engineered trimeric ACE2 proteins and showed T-ACE2, AH5, GGGH5, H3, H4, H6, H7, AP12, AP15, M1, M2, M3, M4, M5, AP15-Cut, M1-Cut, M2-Cut, M3-Cut, M4-Cut and M5-Cut can bind spike protein with extremely high affinity to potently inhibit all tested viruses including SARS-CoV-2, SARS-CoV, eight naturally occurred SARS-CoV-2 mutants and a SARSr-CoV. We demonstrated T-ACE2 can induce spike protein to transit to the unique three RBDs up conformation and bind all three RBDs simultaneously. The rigid linker employed in T-ACE2 has been injected into mice and didn't seem to show strong immunogenicity (50), 3HB and foldon trimerization motifs have been observed to cause immunogenicity, but introducing glycans can silence the immunogenicity without disrupting the trimer formation (51). We believe proteins engineered based wild type ACE2 such as T-ACE2 would be the most broadly SARS-CoVs neutralizing proteins and will be most resistant to mutational escape. We speculate properly designed higher oligomeric ACE2s may also have additional inter-molecular avidity binding with spike proteins on virus surface thus may have even higher virus inhibition activities. The extremely high binding affinity between T-ACE2 and spike protein (K_{D}<1pM) suggests T-ACE2 could be useful for virus detection methods development. The nature that T-ACE2 was engineered based on native ACE2 sequence also makes such detection methods widely useful for all SARS-CoVs and related viruses.

Whether this T-ACE2 induced spike protein conformation change represents a transition state during virus infection cannot be definitively answered here. Full length ACE2 protein functions as dimer (44). The two monomers from this ACE2 dimer are in two-fold symmetry, they are also in close distance (distance between D615 is about 53 Å), so it's hard to imagine this dimeric ACE2 can engage more than one RBD from the same spike protein with current structural understandings. It is though possible cell surface ACE2 dimers might cluster together to induce more RBDs to adopt up conformation and eventually help virus to transit from prefusion state to postfusion state.

### Materials and Methods

### Protein Preparations

To construct trimeric ACE2s, we inserted the linker (GGGGS) 5 (SEQ ID NO: 49), GS(EAAAK)₅GS (SEQ ID NO: 40), AH5 (SEQ ID NO: 41), GGGH5 (SEQ ID NO: 42), H3 (SEQ ID NO: 43), H4 (SEQ ID NO: 44), H6 (SEQ ID NO: 45), H7 (SEQ ID NO: 46), AP12 (SEQ ID NO: 47), AP15 (SEQ ID NO: 48) or GGGS after ACE2 (18-615), followed by trimerization motifs, an HRV3C cleavage sequence, an eGFP tag and a His8 tag. Monomeric ACE2 (SEQ ID NO: 13) was constructed as ACE2 (18-615)-(GGGGS)₅-HRV3C-eGFP-His8 for direct comparison.

The ACE2 peptidase domain (18-615) (derived from full-length ACE2 (accession number: NM_001371415)) (SEQ ID NO: 3) was cloned from the plasmids donated by Peihui Wang's lab, mutants of ACE2 peptidase domain (SEQ ID Nos: 51-55) were constructed in our own lab based on previous publications. The genes of 3HB and foldon were synthesis by Genewiz, Suzhou, China. All the gene fragments were assembled by the Gibson assembly kit (Cat.C112-01, Vazyme). The assembled fragments were subcloned into pEGFP between XhoI and EcoRI respectively. The cloned plasmids were transformed into E.coli DH5α for amplification. Amplified plasmids were extracted using GoldHi EndoFree Plasmid Maxi Kit (Cat. CW2104M, CWBio).

HEK 293F cells (Invitrogen) were cultured in Freestyle medium (Gibco, Lot.2164683) at 37 ° C. under 6% CO₂ in a CRYSTAL shaker (140 rpm). The cells were transiently transfected with the ACE2 plasmids and polyethylenimine (PEI) (Polysciences, Cat.24765-1) when the cell density reached approximately 1.0×10⁶/mL. 1 mg plasmids were premixed with 2.6 mg PEI in 50 ml of fresh medium for 15 minutes before adding to one liter cell culture. The transfected cells were cultured for 96 hours before harvesting.

For purification of ACE2 proteins, the cell supernatants were harvested by centrifugation at 1000 g for 5 minutes. Then the supernatants were loaded on Ni-NTA beads (Smart-Lifesciences, Cat. SA004100), washed with washing buffer (5 mM imidazole, 1× PBS). Proteins were then eluted with elution buffer (50 mM imidazole, 1× PBS).

The eluted proteins were concentrated and subject to size-exclusion chromatography (Superose 6 Increase 10/300 GL, GE Healthcare) in the PBS buffer. The peak fractions were collected and concentrated for further analysis. The protein molecular weight was analyzed by a size exclusion chromatography (AdvanceBio SEC 300Å) in PBS buffer pH 7.4. The standard proteins were purchased from GE. The results are shown in FIG. 6D. It can be seen that ACE2 monomers having a trimerization domain all associated into ACE2 trimers.

To remove C-terminal tags of ACE2 proteins, 16 ug HRV3C protease (expressed and purified in house) was add to lmg ACE2 protein and incubated at 4 ° C. overnight, followed by size-exclusion chromatography (Superose 6 Increase 10/300 GL, GE Healthcare) purification and analysis .

4-12% SDS-PAGE gels or 12% SDS-PAGE gels were purchased from Genscript (Suzhou). Protein gels were run at 80 V for 5 minutes then turn to 130 V for 45 minutes in 1× MOPS buffer. When the electrophoresis was finished, the protein gels were stained in staining buffer (1.25 grams coomassie Blue R-25 dissolved in 1 L buffer containing 300 ml ethanol, 100 mL acetic acid, and 600 mL water) for 30 minutes. Then the stained gels were destained in destaining buffer (1 L containing 300mL ethanol, 100 mL acetic acid, and 600 mL water) for 2 hours.

### Binding Affinity Measurement using ELISA Assays

### Determination of Optimal S-ECD Loading

96-well ELISA plates (JET BIOFIL, #FEP-100-096) were coated with 50 μL per well of different S-ECD protein concentrations (FIG. 7) in coating buffer (NCM Biotech, #F30500) overnight at 4° C. Plates were washed with phosphate-buffered saline with 0.1% Tween-20 (PBST) four times then blocked with 2% bovine serum albumin (BSA, SIGMA, #B2064-50G) in PBST for 2 hours at room temperature. After blocking, the plates were washed with PBST four times then incubated with 70 μL per well of ACE2 monomer in PBST for 2 hours at 37° C. Plates were washed with PBST four times then incubated with 70 μL per well of 1:2,000 dilution of Anti-GFP antibody (Rabbit PAb, Sino Biological, #13105-RP01) for 1 h at 37° C. Plates were again washed four times then incubated with 70 μL per well of 1:10,000 dilution of HRP-conjugated Goat Anti-Rabbit IgG (Beyotime, #A0208) for 1 hour at 37° C. After final four times washing, plates were added with 100 μL per well of TMB single-component substrate solution (Solarbio, #PR1200) and the reaction was stopped by the addition of 50 μLper well of 1M hydrochloric acid. The absorbance at 450 nm was measured on a Microplate reader (Thermo, Varioskan LUX). From this experiment, we decided to load 3 μg/mL S-ECD for ACE2 proteins binding measurement.

To determine the binding affinities of different ACE2 proteins, 96-well ELISA plates (JET BIOFIL, #1-EP-100-096) were coated with 50 μL per well of S-ECD (3 μg/mL) in coating buffer (NCM Biotech, #F30500) overnight at 4° C. Plates were washed with phosphate-buffered saline with 0.1% Tween-20 (PBST) four times then blocked with 2% bovine serum albumin (BSA, SIGMA, #B2064-50G) in PBST for 2 hours at room temperature. After blocking, the plates were washed with PBST four times then incubated with 70 μL per well of series diluted ACE2 samples in PBST for 2 hours at 37° C. Plates were washed with PBST four times then incubated with 70 μL per well of 1:2,000 dilution of Anti-GFP antibody (Rabbit PAb, Sino Biological, #13105-RP01) for 1 hour at 37° C. Plates were again washed four times then incubated with 70 μL per well of 1:10,000 dilution of HRP-conjugated Goat Anti-Rabbit IgG (Beyotime, #A0208) for 1 hour at 37° C. After final four times washing, plates were added with 100 μL per well of TMB single-component substrate solution (Solarbio, #PR1200) and the reaction was stopped by the addition of 50 μL per well of 1M hydrochloric acid. The absorbance at 450 nm was measured on a Microplate reader (Thermo, Varioskan LUX).

### Binding Affinity Determination Using Bio-Layer Interferometry (BLI)

### Protein Biotinylation

Purified S-ECD protein was biotinylated at a theoretical 1:3 molar ratio with EZ-Link NHS-PEG12-Biotin (Thermo Fisher Scientific, CAT#: 21313) according to the manufacturer's instructions. The unreacted biotin was removed by ultrafiltration with an Amicon column (30 KDa MWCO, Millipore, CAT: UFC5010BK).

### Kinetic Analyses

For kinetic analyses, S-ECD was captured on streptavidin biosensors. Biotinylated S-ECD was diluted to 20 μL g/mL in dilution buffer (PBS with 0.02% Tween 20 and 0.1% BSA). Then sensors baseline were equilibrated in the dilution buffer for 90 seconds. Then the S-ECD was loaded until the thickness signal is 0.6 nm or 0.3 nm (low loading). After loading, the sensor was washed for 60 seconds in the dilution buffer. The sensors were then immersed into wells containing ACE2 proteins for 100 seconds (association phase), followed by immersion in dilution buffers for an additional 300 seconds (dissociation phase). The background signal was measured using an reference sensor with S-ECD loading but no ACE2 protein binding and was subtracted from corresponding ACE2 binding sensor. Curve fitting was performed using a 1:1 binding model and the ForteBio data analysis software. Mean kon, koff values were determined by averaging all binding curves that matched the theoretical fit with an R2 value of 0.95.

### Pseudotyped Virus Inhibition

### Cell Lines

Human hepatoma Huh-7 cells were purchased from the Cell Bank of the Chinese Academy of Science (Shanghai, China). Human colorectal adenocarcinoma Caco-2 cells were obtained from the American Type Culture Collection (ATCC). Human primary embryonic kidney cells (293T) (CRL-3216™) were obtained from the American Type Culture Collection (ATCC). These cells were cultured with Dulbecco's Modified Eagle's Medium (DMEM) containing 10% Fetal bovine serum (FBS), 100 mg/mL streptomycin, and 100 U/mL penicillin at 37° C. under 5% CO₂.

### Plasmid Construction

The envelop-encoding plasmids of SARS-CoV-2-S, SARS-CoV-S, and SARSr-CoV-S (Rs3367 and WIV1) and luciferase-expressing vector (pNL4-3.Luc.R-E-) were maintained in house. The plasmids encoding mutant SARS-CoV-2-S (V341I, F342L, V367F, R4081, A435S, G476S, V483A, or D614G) were constructed using a site mutation kit (Yeasen, China) and confirmed by sequencing.

### Packaging Pseudotyped SARS-CoV-2, Mutant SARS-CoV-2, SARS-CoV, and SARSr-CoV

These pseudoviruses were generated according to the previous study (52, 53). Briefly, the envelop-encoding plasmid (20 μg) and pNL4-3.Luc.R-E- (10 μg) were co-transfected into 293T cells cultured at 10 cm cell culture dish using Vigofect transfection reagent (Vigorous Biotechnology, China). After 10 hours, the cell culture medium was changed with fresh DMEM containing 10% FBS. Supernatants containing pseudovirus were harvested 48 hours later, filtered with 0.45 μm filter (Millipore), and using for single-cycle infection.

### Inhibition of Pseudotyped SARS-CoV-2, Mutant SARS-CoV-2, SARS-CoV, and SARSr-CoV

The pseudovirus inhibition assay was conducted as previously described (52, 53). Briefly, 1×10⁴ Huh-7 cells (or Caco-2 cells) were seeded into the 96-well cell culture plate and cultured for 12 hours. The recombinant proteins (ACE2 trimers) were diluted with DMEM and mixed with pseudovirus, incubated at 37° C. for 30 minutes, and added to Huh-7 cells (or Caco-2 cells). After 12 hours of infection, the culture medium was replaced with fresh DMDM containing 10% FBS, and cells were cultured for an additional 48 hours. Then cells were lysed with Cell Lysis Buffer (Promega, Madison, WI, USA), and the luciferase activity was detected using the Luciferase Assay System (Promega, Madison, WI, USA).

### Authentic SARS-CoV-2 Virus Inhibition

Cell lines and Virus

African green monkey kidney Vero-E6 cell line was cultured with Dulbecco's Modified Eagle's Medium (DMEM) containing 10% Fetal bovine serum (FBS), 100 mg/mL streptomycin, and 100 U/mL penicillin at 37° C. under 5% CO₂. SARS-CoV-2 (SARS-CoV-2/SH01/human/2020/CHN, GenBank No. MT121215) was isolated from a COVID-19 patient in Shanghai, China. The virus was purified and propagated in Vero-E6 cells, then stocked at −80° C. Viral titer was measured by the 50% Tissue culture infective dose (TCID50) method. All experiments involving live SARS-CoV-2 virus were performed in Biosafety Level 3 Laboratory (BSL-3), Fudan University.

### Inhibition of Live SARS-CoV-2 Infection

The live SARS-CoV-2 inhibition assay was performed as previously described(54). Briefly, 3×10⁴ Vero-E6 cells were seeded into the 96-well cell culture plate and cultured for 12 hours. Recombinant proteins (ACE2 trimers) were diluted with FBS-free DMEM and mixed with 100 TCID₅₀ SARS-CoV-2, incubated at 37° C. for 30 minutes. Then, the protein-virus mixtures were added to Vero-E6 cells and incubated at 37° C. for 1 hour. After removing the mixtures, cells were cultured with fresh DMEM containing 2% FBS for a further 48 hours. Then, the supernatants were collected to detect viral RNA titer.

RNA extraction and Quantitative Real-Time PCR (qPCR) Assay

Total viral RNA in supernatants were extracted using Trizol LS reagent (Invitrogen, USA) according to manufacturer's manual. Then qPCR was conducted with a One-Step PrimeScrip RT-PCR Kit (Takara, Japan) following the manufacturer's instructions. qPCR reaction was performed with the program of 95° C. for 10 seconds, 42° C. for 5 minutes; 40 cycles of 95° C. for 5 seconds, 50° C. for 30 seconds, 72° C. for 30 seconds on Bio-Rad CFX96. Viral loads were determined by a standard curve prepared with a plasmid containing SARS-CoV-2 nucleocapsid protein (N) gene (purchased form BGI, China). Primers and probe targeting SARS-CoV-2 N gene were ordered from Genewiz (Suzhou, China) and the sequences as follows:

SARS-CoV-2-N-F:
GGGGAACTTCTCCTGCTAGAAT,
SARS-CoV-2-N-R:
CAGACATTTTGCTCTCAAGCTG,
SARS-CoV-2-N-probe:
5′-FAM-TTGCTGCTGCTTGACAGATT-TAMRA-3′.

### Cryo-EM Sample Preparation

The purification of the extracellular domain (ECD) (Genebank ID: QHD43416.1) (1-1208 aa) of S protein was as previously (55). The purified S-ECD was mixed with the T-ACE2 at a molar ratio of about 1:2 for one hour at 4° C. To remove excessive T-ACE2, the mixture was subjected to size-exclusion chromatography (Superose 6 Increase 10/300 GL, GE Healthcare) in buffer containing 25 mM Tris (pH 8.0), 150 mM NaCl. Peak fractions of S-ECD in complex with T-ACE2 were collected for EM analysis.

The peak fractions of the complex were concentrated to about 1.5 mg/mL and mixed with 0.05% Octyl Maltoside, Fluorinated (Anatrace) before applied to the grids. Aliquots (3.3 μL) of the protein complex were placed on glow-discharged holey carbon grids (Quantifoil Au R1.2/1.3). The grids were blotted for 2.5 s or 3.0 s and flash-frozen in liquid ethane cooled by liquid nitrogen with Vitrobot (Mark IV, Thermo Scientific). The cryo-EM samples were transferred to a Titan Krios operating at 300 kV equipped with Cs corrector, Gatan K3 Summit detector and GIF Quantum energy filter. Movie stacks were automatically collected using AutoEMation (56), with a slit width of 20 eV on the energy filter and a defocus range from −1.2 μm to −2.2 μm in super-resolution mode at a nominal magnification of 81,000x. Each stack was exposed for 2.56 s with an exposure time of 0.08 s per frame, resulting in a total of 32 frames per stack. The total dose rate was approximately 50 e⁻/Å² for each stack. The stacks were motion corrected with MotionCor2 (57) and binned 2-fold, resulting in a pixel size of 1.087 Å/pixel. Meanwhile, dose weighting was performed (58). The defocus values were estimated with Gctf (59).

### Data Processing

Particles were automatically picked using Relion 3.0.6 (60-63) from manually selected micrographs. After 2D classification with Relion, good particles were selected and subject to two cycle of heterogeneous refinement without symmetry using cryoSPARC (64).The good particles were selected and subjected to Non-uniform Refinement (beta) with C1 symmetry, resulting in the 3D reconstruction for the whole structures, which was further subject to 3D classification, 3D auto-refinement and post-processing with Relion. For interface between RBD and ACE2, the datasets were subject to focused refinement with adapted mask on each RBD and ACE2 sub-complex to improve the map quality. Then the dataset of three RBD and ACE2 sub-complexes were combined and subject to focused refinement with Relion, resulting in the 3D reconstruction of better quality on the interface between S-ECD and ACE2.

The resolution was estimated with the gold-standard Fourier shell correlation 0.143 criterion (65) with high-resolution noise substitution (66). Refer to FIGS. 10-12 and Table 1 for details of data collection and processing.

### Model Building and Structure Refinement

For model building of the complex of S-ECD with ACE2, the atomic model of the published structure S-ECD (PDB ID: 7C2L) and ACE2 molecular (PDB ID: 6M18) were used as templates, which were molecular dynamics flexible fitted (MDFF) (67) into the whole cryo-EM map of the complex and the focused-refined cryo-EM map of the RBD-ACE2 sub-complex, respectively. And the fitted atomic models were further manually adjusted with Coot (68). Each residue was manually checked with the chemical properties taken into consideration during model building. Several segments, whose corresponding densities were invisible, were not modeled. Structural refinement was performed in Phenix (69) with secondary structure and geometry restraints to prevent overfitting. To monitor the potential overfitting, the model was refined against one of the two independent half maps from the gold-standard 3D refinement approach. Then, the refined model was tested against the other map. Statistics associated with data collection, 3D reconstruction and model building were summarized in Table 1.

TABLE 1
Cryo-EM data collection and refinement statistics.

| Data collection | | |
|---|---|---|
| EM equipment | Titan Krios (Thermo Fisher Scientific) | |
| Voltage (kV) | 300 | |
| Detector | Gatan K3 Summit | |
| Energy filter | Gatan GIF Quantum, 20 eV slit | |
| Pixel size (Å) | 1.087 | |
| Electron dose (e−/Å2) | 50 | |
| Defocus range (μm) | −1.2~−2.2 | |
| Number of collected | 1,197 | |
| micrographs | | |
| Number of selected micrographs | 1,153 | |
| Sample | SP_T-ACE2 | |
| 3D Reconstruction | | |
| Software | Whole model | Interface between |
| | cryoSPARC/Relion | RBD and ACE2 |
| | | Relion |
| Number of used particles | 57,404 | 59,822 |
| Resolution (Å) | 4.0 | 4.3 |
| Symmetry | C1 | |
| Map sharpening B factor (Å²) | −90 | |
| Refinement | | |
| Software | Phenix | |
| Cell dimensions (Å) | 313.056 | |
| Model composition | | |
| Protein residues | 4,804 | |
| Side chains assigned | 4,804 | |
| Sugar | 104 | |
| R.m.s deviations | | |
| Bonds length (Å) | 0.007 | |
| Bonds Angle (°) | 1.065 | |
| Ramachandran plot | | |
| statistics (%) | | |

| | |
|---|---|
| Preferred | 92.56 |
| Allowed | 7.36 |
| Outlier | 0.08 |

ACE2 1-805
(SEQ ID NO: 1)
MSSSSWLLLSLVAVTAAQSTIEEQAKTFLDKFNHEAEDLFYQSSLASWNYNTNITEENVQNMN
NAGDKWSAFLKEQSTLAQMYPLQEIQNLTVKLQLQALQQNGSSVLSEDKSKRLNTILNTMSTI
YSTGKVCNPDNPQECLLLEPGLNEIMANSLDYNERLWAWESWRSEVGKQLRPLYEEYVVLK
NEMARANHYEDYGDYWRGDYEVNGVDGYDYSRGQLIEDVEHTFEEIKPLYEHLHAYVRAKL
MNAYPSYISPIGCLPAHLLGDMWGRFWTNLYSLTVPFGQKPNIDVTDAMVDQAWDAQRIFKE
AEKFFVSVGLPNMTQGFWENSMLTDPGNVQKAVCHPTAWDLGKGDFRILMCTKVTMDDFLT
AHHEMGHIQYDMAYAAQPFLLRNGANEGFHEAVGEIMSLSAATPKHLKSIGLLSPDFQEDNE
TEINFLLKQALTIVGTLPFTYMLEKWRWMVFKGEIPKDQWMKKWWEMKREIVGVVEPVPHD
ETYCDPASLFHVSNDYSFIRYYTRTLYQFQFQEALCQAAKHEGPLHKCDISNSTEAGQKLFNM
LRLGKSEPWTLALENVVGAKNMNVRPLLNYFEPLFTWLKDQNKNSFVGWSTDWSPYADQSI
KVRISLKSALGDKAYEWNDNEMYLFRSSVAYAMRQYFLKVKNQMILFGEEDVRVANLKPRIS
FNFFVTAPKNVSDIIPRTEVEKAIRMSRSRINDAFRLNDNSLEFLGIQPTLGPPNQPPVSIWLIVF
GVVMGVIVVGIVILIFTGIRDRKKKNKARSGENPYASIDISKGENNPGFQNTDDVQTSF
ACE2 1-740 (full extracellular domain)
(SEQ ID NO: 2)
MSSSSWLLLSLVAVTAAQSTIEEQAKTFLDKFNHEAEDLFYQSSLASWNYNTNITEENVQNMN
NAGDKWSAFLKEQSTLAQMYPLQEIQNLTVKLQLQALQQNGSSVLSEDKSKRLNTILNTMSTI
YSTGKVCNPDNPQECLLLEPGLNEIMANSLDYNERLWAWESWRSEVGKQLRPLYEEYVVLK
NEMARANHYEDYGDYWRGDYEVNGVDGYDYSRGQLIEDVEHTFEEIKPLYEHLHAYVRAKL
MNAYPSYISPIGCLPAHLLGDMWGRFWTNLYSLTVPFGQKPNIDVTDAMVDQAWDAQRIFKE
AEKFFVSVGLPNMTQGFWENSMLTDPGNVQKAVCHPTAWDLGKGDFRILMCTKVTMDDFLT
AHHEMGHIQYDMAYAAQPFLLRNGANEGFHEAVGEIMSLSAATPKHLKSIGLLSPDFQEDNE
TEINFLLKQALTIVGTLPFTYMLEKWRWMVFKGEIPKDQWMKKWWEMKREIVGVVEPVPHD
ETYCDPASLFHVSNDYSFIRYYTRTLYQFQFQEALCQAAKHEGPLHKCDISNSTEAGQKLFNM
LRLGKSEPWTLALENVVGAKNMNVRPLLNYFEPLFTWLKDQNKNSFVGWSTDWSPYADQSI
KVRISLKSALGDKAYEWNDNEMYLFRSSVAYAMRQYFLKVKNQMILFGEEDVRVANLKPRIS
FNFFVTAPKNVSDIIPRTEVEKAIRMSRSRINDAFRLNDNSLEFLGIQPTLGPPNQPPVS
ACE2 18-615 (peptidase domain)
(SEQ ID NO: 3)
QSTIEEQAKTFLDKFNHEAEDLFYQSSLASWNYNTNITEENVQNMNNAGDKWSAFLKEQSTL
AQMYPLQEIQNLTVKLQLQALQQNGSSVLSEDKSKRLNTILNTMSTIYSTGKVCNPDNPQECL
LLEPGLNEIMANSLDYNERLWAWESWRSEVGKQLRPLYEEYVVLKNEMARANHYEDYGDY
WRGDYEVNGVDGYDYSRGQLIEDVEHTFEEIKPLYEHLHAYVRAKLMNAYPSYISPIGCLPAH
LLGDMWGRFWTNLYSLTVPFGQKPNIDVTDAMVDQAWDAQRIFKEAEKFFVSVGLPNMTQG
FWENSMLTDPGNVQKAVCHPTAWDLGKGDFRILMCTKVTMDDFLTAHHEMGHIQYDMAYA
AQPFLLRNGANEGFHEAVGEIMSLSAATPKHLKSIGLLSPDFQEDNETEINFLLKQALTIVGTLP
FTYMLEKWRWMVFKGEIPKDQWMKKWWEMKREIVGVVEPVPHDETYCDPASLFHVSNDYS
FIRYYTRTLYQFQFQEALCQAAKHEGPLHKCDISNSTEAGQKLFNMLRLGKSEPWTLALENVV
GAKNMNVRPLLNYFEPLFTWLKDQNKNSFVGWSTDWSPYAD
ACE2-rigid-Foldon DNA Sequence
(SEQ ID NO: 4)
CAGTCCACCATTGAGGAACAGGCCAAGACATTTTTGGACAAGTTTAACCACGAAGCCGAA
GACCTGTTCTATCAAAGTTCACTTGCTTCTTGGAATTATAACACCAATATTACTGAAGAGA
ATGTCCAAAACATGAATAATGCTGGGGACAAATGGTCTGCCTTTTTAAAGGAACAGTCCA
CACTTGCCCAAATGTATCCACTACAAGAAATTCAGAATCTCACAGTCAAGCTTCAGCTGCA
GGCTCTTCAGCAAAATGGGTCTTCAGTGCTCTCAGAAGACAAGAGCAAACGGTTGAACAC
AATTCTAAATACAATGAGCACCATCTACAGTACTGGAAAAGTTTGTAACCCAGATAATCC
ACAAGAATGCTTATTACTTGAACCAGGTTTGAATGAAATAATGGCAAACAGTTTAGACTA
CAATGAGAGGCTCTGGGCTTGGGAAAGCTGGAGATCTGAGGTCGGCAAGCAGCTGAGGC
CATTATATGAAGAGTATGTGGTCTTGAAAAATGAGATGGCAAGAGCAAATCATTATGAGG
ACTATGGGGATTATTGGAGAGGAGACTATGAAGTAAATGGGGTAGATGGCTATGACTACA
GCCGCGGCCAGTTGATTGAAGATGTGGAACATACCTTTGAAGAGATTAAACCATTATATG
AACATCTTCATGCCTATGTGAGGGCAAAGTTGATGAATGCCTATCCTTCCTATATCAGTCC
AATTGGATGCCTCCCTGCTCATTTGCTTGGTGATATGTGGGGTAGATTTTGGACAAATCTG
TACTCTTTGACAGTTCCCTTTGGACAGAAACCAAACATAGATGTTACTGATGCAATGGTGG
ACCAGGCCTGGGATGCACAGAGAATATTCAAGGAGGCCGAGAAGTTCTTTGTATCTGTTG
GTCTTCCTAATATGACTCAAGGATTCTGGGAAAATTCCATGCTAACGGACCCAGGAAATG
TTCAGAAAGCAGTCTGCCATCCCACAGCTTGGGACCTGGGGAAGGGCGACTTCAGGATCC
TTATGTGCACAAAGGTGACAATGGACGACTTCCTGACAGCTCATCATGAGATGGGGCATA
TCCAGTATGATATGGCATATGCTGCACAACCTTTTCTGCTAAGAAATGGAGCTAATGAAG
GATTCCATGAAGCTGTTGGGGAAATCATGTCACTTTCTGCAGCCACACCTAAGCATTTAAA
ATCCATTGGTCTTCTGTCACCCGATTTTCAAGAAGACAATGAAACAGAAATAAACTTCCTG
CTCAAACAAGCACTCACGATTGTTGGGACTCTGCCATTTACTTACATGTTAGAGAAGTGGA
GGTGGATGGTCTTTAAAGGGGAAATTCCCAAAGACCAGTGGATGAAAAAGTGGTGGGAG
ATGAAGCGAGAGATAGTTGGGGTGGTGGAACCTGTGCCCCATGATGAAACATACTGTGAC
CCCGCATCTCTGTTCCATGTTTCTAATGATTACTCATTCATTCGATATTACACAAGGACCCT
TTACCAATTCCAGTTTCAAGAAGCACTTTGTCAAGCAGCTAAACATGAAGGCCCTCTGCAC
AAATGTGACATCTCAAACTCTACAGAAGCTGGACAGAAACTGTTCAATATGCTGAGGCTT
GGAAAATCAGAACCCTGGACCCTAGCATTGGAAAATGTTGTAGGAGCAAAGAACATGAA
TGTAAGGCCACTGCTCAACTACTTTGAGCCCTTATTTACCTGGCTGAAAGACCAGAACAAG
AATTCTTTTGTGGGATGGAGTACCGACTGGAGTCCATATGCAGACGGCAGCGAAGCGGCG
GCGAAAGAAGCGGCGGCGAAAGAAGCGGCGGCGAAAGAAGCGGCGGCGAAAGAAGCGG
CGGCGAAAGGAAGCGGCTACATCCCCGAGGCCCCCAGGGACGGCCAGGCCTACGTGAGG
AAGGACGGCGAGTGGGTGCTGCTGAGCACCTTCCTGGGCAGC
ACE2-rigid-Foldon Protein Sequence
(SEQ ID NO: 5)
QSTIEEQAKTFLDKFNHEAEDLFYQSSLASWNYNTNITEENVQNMNNAGDKWSAFLKEQSTL
AQMYPLQEIQNLTVKLQLQALQQNGSSVLSEDKSKRLNTILNTMSTIYSTGKVCNPDNPQECL
LLEPGLNEIMANSLDYNERLWAWESWRSEVGKQLRPLYEEYVVLKNEMARANHYEDYGDY
WRGDYEVNGVDGYDYSRGQLIEDVEHTFEEIKPLYEHLHAYVRAKLMNAYPSYISPIGCLPAH
LLGDMWGRFWTNLYSLTVPFGQKPNIDVTDAMVDQAWDAQRIFKEAEKFFVSVGLPNMTQG
FWENSMLTDPGNVQKAVCHPTAWDLGKGDFRILMCTKVTMDDFLTAHHEMGHIQYDMAYA
AQPFLLRNGANEGFHEAVGEIMSLSAATPKHLKSIGLLSPDFQEDNETEINFLLKQALTIVGTLP
FTYMLEKWRWMVFKGEIPKDQWMKKWWEMKREIVGVVEPVPHDETYCDPASLFHVSNDYS
FIRYYTRTLYQFQFQEALCQAAKHEGPLHKCDISNSTEAGQKLFNMLRLGKSEPWTLALENVV
GAKNMNVRPLLNYFEPLFTWLKDQNKNSFVGWSTDWSPYADGSEAAAKEAAAKEAAAKEA
AAKEAAAKGSGYIPEAPRDGQAYVRKDGEWVLLSTFLGS
ACE2-rigid-3HB DNA Sequence
(SEQ ID NO: 6)
CAGTCCACCATTGAGGAACAGGCCAAGACATTTTTGGACAAGTTTAACCACGAAGCCGAA
GACCTGTTCTATCAAAGTTCACTTGCTTCTTGGAATTATAACACCAATATTACTGAAGAGA
ATGTCCAAAACATGAATAATGCTGGGGACAAATGGTCTGCCTTTTTAAAGGAACAGTCCA
CACTTGCCCAAATGTATCCACTACAAGAAATTCAGAATCTCACAGTCAAGCTTCAGCTGCA
GGCTCTTCAGCAAAATGGGTCTTCAGTGCTCTCAGAAGACAAGAGCAAACGGTTGAACAC
AATTCTAAATACAATGAGCACCATCTACAGTACTGGAAAAGTTTGTAACCCAGATAATCC
ACAAGAATGCTTATTACTTGAACCAGGTTTGAATGAAATAATGGCAAACAGTTTAGACTA
CAATGAGAGGCTCTGGGCTTGGGAAAGCTGGAGATCTGAGGTCGGCAAGCAGCTGAGGC
CATTATATGAAGAGTATGTGGTCTTGAAAAATGAGATGGCAAGAGCAAATCATTATGAGG
ACTATGGGGATTATTGGAGAGGAGACTATGAAGTAAATGGGGTAGATGGCTATGACTACA
GCCGCGGCCAGTTGATTGAAGATGTGGAACATACCTTTGAAGAGATTAAACCATTATATG
AACATCTTCATGCCTATGTGAGGGCAAAGTTGATGAATGCCTATCCTTCCTATATCAGTCC
AATTGGATGCCTCCCTGCTCATTTGCTTGGTGATATGTGGGGTAGATTTTGGACAAATCTG
TACTCTTTGACAGTTCCCTTTGGACAGAAACCAAACATAGATGTTACTGATGCAATGGTGG
ACCAGGCCTGGGATGCACAGAGAATATTCAAGGAGGCCGAGAAGTTCTTTGTATCTGTTG
GTCTTCCTAATATGACTCAAGGATTCTGGGAAAATTCCATGCTAACGGACCCAGGAAATG
TTCAGAAAGCAGTCTGCCATCCCACAGCTTGGGACCTGGGGAAGGGCGACTTCAGGATCC
TTATGTGCACAAAGGTGACAATGGACGACTTCCTGACAGCTCATCATGAGATGGGGCATA
TCCAGTATGATATGGCATATGCTGCACAACCTTTTCTGCTAAGAAATGGAGCTAATGAAG
GATTCCATGAAGCTGTTGGGGAAATCATGTCACTTTCTGCAGCCACACCTAAGCATTTAAA
ATCCATTGGTCTTCTGTCACCCGATTTTCAAGAAGACAATGAAACAGAAATAAACTTCCTG
CTCAAACAAGCACTCACGATTGTTGGGACTCTGCCATTTACTTACATGTTAGAGAAGTGGA
GGTGGATGGTCTTTAAAGGGGAAATTCCCAAAGACCAGTGGATGAAAAAGTGGTGGGAG
ATGAAGCGAGAGATAGTTGGGGTGGTGGAACCTGTGCCCCATGATGAAACATACTGTGAC
CCCGCATCTCTGTTCCATGTTTCTAATGATTACTCATTCATTCGATATTACACAAGGACCCT
TTACCAATTCCAGTTTCAAGAAGCACTTTGTCAAGCAGCTAAACATGAAGGCCCTCTGCAC
AAATGTGACATCTCAAACTCTACAGAAGCTGGACAGAAACTGTTCAATATGCTGAGGCTT
GGAAAATCAGAACCCTGGACCCTAGCATTGGAAAATGTTGTAGGAGCAAAGAACATGAA
TGTAAGGCCACTGCTCAACTACTTTGAGCCCTTATTTACCTGGCTGAAAGACCAGAACAAG
AATTCTTTTGTGGGATGGAGTACCGACTGGAGTCCATATGCAGACGGCAGCGAAGCGGCG
GCGAAAGAAGCGGCGGCGAAAGAAGCGGCGGCGAAAGAAGCGGCGGCGAAAGAAGCGG
CGGCGAAAGGAAGCGGCGAGATCGCCGCCATCAAGCAGGAGATCGCCGCCATCAAGAAG
GAGATCGCCGCCATCAAGTGGGAGATCGCCGCCATCAAGCAGGGCTACGGC
ACE2-rigid-3HB Protein Sequence
(SEQ ID NO: 7)
QSTIEEQAKTFLDKFNHEAEDLFYQSSLASWNYNTNITEENVQNMNNAGDKWSAFLKEQSTL
AQMYPLQEIQNLTVKLQLQALQQNGSSVLSEDKSKRLNTILNTMSTIYSTGKVCNPDNPQECL
LLEPGLNEIMANSLDYNERLWAWESWRSEVGKQLRPLYEEYVVLKNEMARANHYEDYGDY
WRGDYEVNGVDGYDYSRGQLIEDVEHTFEEIKPLYEHLHAYVRAKLMNAYPSYISPIGCLPAH
LLGDMWGRFWTNLYSLTVPFGQKPNIDVTDAMVDQAWDAQRIFKEAEKFFVSVGLPNMTQG
FWENSMLTDPGNVQKAVCHPTAWDLGKGDFRILMCTKVTMDDFLTAHHEMGHIQYDMAYA
AQPFLLRNGANEGFHEAVGEIMSLSAATPKHLKSIGLLSPDFQEDNETEINFLLKQALTIVGTLP
FTYMLEKWRWMVFKGEIPKDQWMKKWWEMKREIVGVVEPVPHDETYCDPASLFHVSNDYS
FIRYYTRTLYQFQFQEALCQAAKHEGPLHKCDISNSTEAGQKLFNMLRLGKSEPWTLALENVV
GAKNMNVRPLLNYFEPLFTWLKDQNKNSFVGWSTDWSPYADGSEAAAKEAAAKEAAAKEA
AAKEAAAKGSGEIAAIKQEIAAIKKEIAAIKWEIAAIKQGYG
ACE2-flexible-foldon DNA Sequence
(SEQ ID NO: 8)
CAGTCCACCATTGAGGAACAGGCCAAGACATTTTTGGACAAGTTTAACCACGAAGCCGAA
GACCTGTTCTATCAAAGTTCACTTGCTTCTTGGAATTATAACACCAATATTACTGAAGAGA
ATGTCCAAAACATGAATAATGCTGGGGACAAATGGTCTGCCTTTTTAAAGGAACAGTCCA
CACTTGCCCAAATGTATCCACTACAAGAAATTCAGAATCTCACAGTCAAGCTTCAGCTGCA
GGCTCTTCAGCAAAATGGGTCTTCAGTGCTCTCAGAAGACAAGAGCAAACGGTTGAACAC
AATTCTAAATACAATGAGCACCATCTACAGTACTGGAAAAGTTTGTAACCCAGATAATCC
ACAAGAATGCTTATTACTTGAACCAGGTTTGAATGAAATAATGGCAAACAGTTTAGACTA
CAATGAGAGGCTCTGGGCTTGGGAAAGCTGGAGATCTGAGGTCGGCAAGCAGCTGAGGC
CATTATATGAAGAGTATGTGGTCTTGAAAAATGAGATGGCAAGAGCAAATCATTATGAGG
ACTATGGGGATTATTGGAGAGGAGACTATGAAGTAAATGGGGTAGATGGCTATGACTACA
GCCGCGGCCAGTTGATTGAAGATGTGGAACATACCTTTGAAGAGATTAAACCATTATATG
AACATCTTCATGCCTATGTGAGGGCAAAGTTGATGAATGCCTATCCTTCCTATATCAGTCC
AATTGGATGCCTCCCTGCTCATTTGCTTGGTGATATGTGGGGTAGATTTTGGACAAATCTG
TACTCTTTGACAGTTCCCTTTGGACAGAAACCAAACATAGATGTTACTGATGCAATGGTGG
ACCAGGCCTGGGATGCACAGAGAATATTCAAGGAGGCCGAGAAGTTCTTTGTATCTGTTG
GTCTTCCTAATATGACTCAAGGATTCTGGGAAAATTCCATGCTAACGGACCCAGGAAATG
TTCAGAAAGCAGTCTGCCATCCCACAGCTTGGGACCTGGGGAAGGGCGACTTCAGGATCC
TTATGTGCACAAAGGTGACAATGGACGACTTCCTGACAGCTCATCATGAGATGGGGCATA
TCCAGTATGATATGGCATATGCTGCACAACCTTTTCTGCTAAGAAATGGAGCTAATGAAG
GATTCCATGAAGCTGTTGGGGAAATCATGTCACTTTCTGCAGCCACACCTAAGCATTTAAA
ATCCATTGGTCTTCTGTCACCCGATTTTCAAGAAGACAATGAAACAGAAATAAACTTCCTG
CTCAAACAAGCACTCACGATTGTTGGGACTCTGCCATTTACTTACATGTTAGAGAAGTGGA
GGTGGATGGTCTTTAAAGGGGAAATTCCCAAAGACCAGTGGATGAAAAAGTGGTGGGAG
ATGAAGCGAGAGATAGTTGGGGTGGTGGAACCTGTGCCCCATGATGAAACATACTGTGAC
CCCGCATCTCTGTTCCATGTTTCTAATGATTACTCATTCATTCGATATTACACAAGGACCCT
TTACCAATTCCAGTTTCAAGAAGCACTTTGTCAAGCAGCTAAACATGAAGGCCCTCTGCAC
AAATGTGACATCTCAAACTCTACAGAAGCTGGACAGAAACTGTTCAATATGCTGAGGCTT
GGAAAATCAGAACCCTGGACCCTAGCATTGGAAAATGTTGTAGGAGCAAAGAACATGAA
TGTAAGGCCACTGCTCAACTACTTTGAGCCCTTATTTACCTGGCTGAAAGACCAGAACAAG
AATTCTTTTGTGGGATGGAGTACCGACTGGAGTCCATATGCAGACGGTGGAGGAGGTTCT
GGCGGAGGAGGTAGTGGCGGAGGAGGTTCAGGAGGCGGCGGAAGCGGTGGAGGAGGTTC
TGGCTACATCCCCGAGGCCCCCAGGGACGGCCAGGCCTACGTGAGGAAGGACGGCGAGT
GGGTGCTGCTGAGCACCTTCCTG
ACE2-flexible-foldon Protein Sequence
(SEQ ID NO: 9)
QSTIEEQAKTFLDKFNHEAEDLFYQSSLASWNYNTNITEENVQNMNNAGDKWSAFLKEQSTL
AQMYPLQEIQNLTVKLQLQALQQNGSSVLSEDKSKRLNTILNTMSTIYSTGKVCNPDNPQECL
LLEPGLNEIMANSLDYNERLWAWESWRSEVGKQLRPLYEEYVVLKNEMARANHYEDYGDY
WRGDYEVNGVDGYDYSRGQLIEDVEHTFEEIKPLYEHLHAYVRAKLMNAYPSYISPIGCLPAH
LLGDMWGRFWTNLYSLTVPFGQKPNIDVTDAMVDQAWDAQRIFKEAEKFFVSVGLPNMTQG
FWENSMLTDPGNVQKAVCHPTAWDLGKGDFRILMCTKVTMDDFLTAHHEMGHIQYDMAYA
AQPFLLRNGANEGFHEAVGEIMSLSAATPKHLKSIGLLSPDFQEDNETEINFLLKQALTIVGTLP
FTYMLEKWRWMVFKGEIPKDQWMKKWWEMKREIVGVVEPVPHDETYCDPASLFHVSNDYS
FIRYYTRTLYQFQFQEALCQAAKHEGPLHKCDISNSTEAGQKLFNMLRLGKSEPWTLALENVV
GAKNMNVRPLLNYFEPLFTWLKDQNKNSFVGWSTDWSPYADGGGGSGGGGSGGGGSGGGG
SGGGGSGYIPEAPRDGQAYVRKDGEWVLLSTFL
ACE2-flexible-3HB DNA Sequence
(SEQ ID NO: 10)
CAGTCCACCATTGAGGAACAGGCCAAGACATTTTTGGACAAGTTTAACCACGAAGCCGAA
GACCTGTTCTATCAAAGTTCACTTGCTTCTTGGAATTATAACACCAATATTACTGAAGAGA
ATGTCCAAAACATGAATAATGCTGGGGACAAATGGTCTGCCTTTTTAAAGGAACAGTCCA
CACTTGCCCAAATGTATCCACTACAAGAAATTCAGAATCTCACAGTCAAGCTTCAGCTGCA
GGCTCTTCAGCAAAATGGGTCTTCAGTGCTCTCAGAAGACAAGAGCAAACGGTTGAACAC
AATTCTAAATACAATGAGCACCATCTACAGTACTGGAAAAGTTTGTAACCCAGATAATCC
ACAAGAATGCTTATTACTTGAACCAGGTTTGAATGAAATAATGGCAAACAGTTTAGACTA
CAATGAGAGGCTCTGGGCTTGGGAAAGCTGGAGATCTGAGGTCGGCAAGCAGCTGAGGC
CATTATATGAAGAGTATGTGGTCTTGAAAAATGAGATGGCAAGAGCAAATCATTATGAGG
ACTATGGGGATTATTGGAGAGGAGACTATGAAGTAAATGGGGTAGATGGCTATGACTACA
GCCGCGGCCAGTTGATTGAAGATGTGGAACATACCTTTGAAGAGATTAAACCATTATATG
AACATCTTCATGCCTATGTGAGGGCAAAGTTGATGAATGCCTATCCTTCCTATATCAGTCC
AATTGGATGCCTCCCTGCTCATTTGCTTGGTGATATGTGGGGTAGATTTTGGACAAATCTG
TACTCTTTGACAGTTCCCTTTGGACAGAAACCAAACATAGATGTTACTGATGCAATGGTGG
ACCAGGCCTGGGATGCACAGAGAATATTCAAGGAGGCCGAGAAGTTCTTTGTATCTGTTG
GTCTTCCTAATATGACTCAAGGATTCTGGGAAAATTCCATGCTAACGGACCCAGGAAATG
TTCAGAAAGCAGTCTGCCATCCCACAGCTTGGGACCTGGGGAAGGGCGACTTCAGGATCC
TTATGTGCACAAAGGTGACAATGGACGACTTCCTGACAGCTCATCATGAGATGGGGCATA
TCCAGTATGATATGGCATATGCTGCACAACCTTTTCTGCTAAGAAATGGAGCTAATGAAG
GATTCCATGAAGCTGTTGGGGAAATCATGTCACTTTCTGCAGCCACACCTAAGCATTTAAA
ATCCATTGGTCTTCTGTCACCCGATTTTCAAGAAGACAATGAAACAGAAATAAACTTCCTG
CTCAAACAAGCACTCACGATTGTTGGGACTCTGCCATTTACTTACATGTTAGAGAAGTGGA
GGTGGATGGTCTTTAAAGGGGAAATTCCCAAAGACCAGTGGATGAAAAAGTGGTGGGAG
ATGAAGCGAGAGATAGTTGGGGTGGTGGAACCTGTGCCCCATGATGAAACATACTGTGAC
CCCGCATCTCTGTTCCATGTTTCTAATGATTACTCATTCATTCGATATTACACAAGGACCCT
TTACCAATTCCAGTTTCAAGAAGCACTTTGTCAAGCAGCTAAACATGAAGGCCCTCTGCAC
AAATGTGACATCTCAAACTCTACAGAAGCTGGACAGAAACTGTTCAATATGCTGAGGCTT
GGAAAATCAGAACCCTGGACCCTAGCATTGGAAAATGTTGTAGGAGCAAAGAACATGAA
TGTAAGGCCACTGCTCAACTACTTTGAGCCCTTATTTACCTGGCTGAAAGACCAGAACAAG
AATTCTTTTGTGGGATGGAGTACCGACTGGAGTCCATATGCAGACGGTGGAGGAGGTTCT
GGCGGAGGAGGTAGTGGCGGAGGAGGTTCAGGAGGCGGCGGAAGCGGTGGAGGAGGTTC
TGGCGAGATCGCCGCCATCAAGCAGGAGATCGCCGCCATCAAGAAGGAGATCGCCGCCAT
CAAGTGGGAGATCGCCGCCATCAAGCAGGGCTACGGC
ACE2-flexible-3HB Protein Sequence
(SEQ ID NO: 11)
QSTIEEQAKTFLDKFNHEAEDLFYQSSLASWNYNTNITEENVQNMNNAGDKWSAFLKEQSTL
AQMYPLQEIQNLTVKLQLQALQQNGSSVLSEDKSKRLNTILNTMSTIYSTGKVCNPDNPQECL
LLEPGLNEIMANSLDYNERLWAWESWRSEVGKQLRPLYEEYVVLKNEMARANHYEDYGDY
WRGDYEVNGVDGYDYSRGQLIEDVEHTFEEIKPLYEHLHAYVRAKLMNAYPSYISPIGCLPAH
LLGDMWGRFWTNLYSLTVPFGQKPNIDVTDAMVDQAWDAQRIFKEAEKFFVSVGLPNMTQG
FWENSMLTDPGNVQKAVCHPTAWDLGKGDFRILMCTKVTMDDFLTAHHEMGHIQYDMAYA
AQPFLLRNGANEGFHEAVGEIMSLSAATPKHLKSIGLLSPDFQEDNETEINFLLKQALTIVGTLP
FTYMLEKWRWMVFKGEIPKDQWMKKWWEMKREIVGVVEPVPHDETYCDPASLFHVSNDYS
FIRYYTRTLYQFQFQEALCQAAKHEGPLHKCDISNSTEAGQKLFNMLRLGKSEPWTLALENVV
GAKNMNVRPLLNYFEPLFTWLKDQNKNSFVGWSTDWSPYADGGGGSGGGGSGGGGSGGGG
SGGGGSGEIAAIKQEIAAIKKEIAAIKWEIAAIKQGYG
Monomeric ACE2 DNA Sequence
(SEQ ID NO: 12)
CAGTCCACCATTGAGGAACAGGCCAAGACATTTTTGGACAAGTTTAACCACGAAGCCGAA
GACCTGTTCTATCAAAGTTCACTTGCTTCTTGGAATTATAACACCAATATTACTGAAGAGA
ATGTCCAAAACATGAATAATGCTGGGGACAAATGGTCTGCCTTTTTAAAGGAACAGTCCA
CACTTGCCCAAATGTATCCACTACAAGAAATTCAGAATCTCACAGTCAAGCTTCAGCTGCA
GGCTCTTCAGCAAAATGGGTCTTCAGTGCTCTCAGAAGACAAGAGCAAACGGTTGAACAC
AATTCTAAATACAATGAGCACCATCTACAGTACTGGAAAAGTTTGTAACCCAGATAATCC
ACAAGAATGCTTATTACTTGAACCAGGTTTGAATGAAATAATGGCAAACAGTTTAGACTA
CAATGAGAGGCTCTGGGCTTGGGAAAGCTGGAGATCTGAGGTCGGCAAGCAGCTGAGGC
CATTATATGAAGAGTATGTGGTCTTGAAAAATGAGATGGCAAGAGCAAATCATTATGAGG
ACTATGGGGATTATTGGAGAGGAGACTATGAAGTAAATGGGGTAGATGGCTATGACTACA
GCCGCGGCCAGTTGATTGAAGATGTGGAACATACCTTTGAAGAGATTAAACCATTATATG
AACATCTTCATGCCTATGTGAGGGCAAAGTTGATGAATGCCTATCCTTCCTATATCAGTCC
AATTGGATGCCTCCCTGCTCATTTGCTTGGTGATATGTGGGGTAGATTTTGGACAAATCTG
TACTCTTTGACAGTTCCCTTTGGACAGAAACCAAACATAGATGTTACTGATGCAATGGTGG
ACCAGGCCTGGGATGCACAGAGAATATTCAAGGAGGCCGAGAAGTTCTTTGTATCTGTTG
GTCTTCCTAATATGACTCAAGGATTCTGGGAAAATTCCATGCTAACGGACCCAGGAAATG
TTCAGAAAGCAGTCTGCCATCCCACAGCTTGGGACCTGGGGAAGGGCGACTTCAGGATCC
TTATGTGCACAAAGGTGACAATGGACGACTTCCTGACAGCTCATCATGAGATGGGGCATA
TCCAGTATGATATGGCATATGCTGCACAACCTTTTCTGCTAAGAAATGGAGCTAATGAAG
GATTCCATGAAGCTGTTGGGGAAATCATGTCACTTTCTGCAGCCACACCTAAGCATTTAAA
ATCCATTGGTCTTCTGTCACCCGATTTTCAAGAAGACAATGAAACAGAAATAAACTTCCTG
CTCAAACAAGCACTCACGATTGTTGGGACTCTGCCATTTACTTACATGTTAGAGAAGTGGA
GGTGGATGGTCTTTAAAGGGGAAATTCCCAAAGACCAGTGGATGAAAAAGTGGTGGGAG
ATGAAGCGAGAGATAGTTGGGGTGGTGGAACCTGTGCCCCATGATGAAACATACTGTGAC
CCCGCATCTCTGTTCCATGTTTCTAATGATTACTCATTCATTCGATATTACACAAGGACCCT
TTACCAATTCCAGTTTCAAGAAGCACTTTGTCAAGCAGCTAAACATGAAGGCCCTCTGCAC
AAATGTGACATCTCAAACTCTACAGAAGCTGGACAGAAACTGTTCAATATGCTGAGGCTT
GGAAAATCAGAACCCTGGACCCTAGCATTGGAAAATGTTGTAGGAGCAAAGAACATGAA
TGTAAGGCCACTGCTCAACTACTTTGAGCCCTTATTTACCTGGCTGAAAGACCAGAACAAG
AATTCTTTTGTGGGATGGAGTACCGACTGGAGTCCATATGCAGACGGTGGAGGAGGTTCT
GGCGGAGGAGGTAGTGGCGGAGGAGGTTCAGGAGGCGGCGGAAGCGGTGGAGGAGGTTC
T
Monomeric ACE2 Protein Sequence
(SEQ ID NO: 13)
QSTIEEQAKTFLDKFNHEAEDLFYQSSLASWNYNTNITEENVQNMNNAGDKWSAFLKEQSTL
AQMYPLQEIQNLTVKLQLQALQQNGSSVLSEDKSKRLNTILNTMSTIYSTGKVCNPDNPQECL
LLEPGLNEIMANSLDYNERLWAWESWRSEVGKQLRPLYEEYVVLKNEMARANHYEDYGDY
WRGDYEVNGVDGYDYSRGQLIEDVEHTFEEIKPLYEHLHAYVRAKLMNAYPSYISPIGCLPAH
LLGDMWGRFWTNLYSLTVPFGQKPNIDVTDAMVDQAWDAQRIFKEAEKFFVSVGLPNMTQG
FWENSMLTDPGNVQKAVCHPTAWDLGKGDFRILMCTKVTMDDFLTAHHEMGHIQYDMAYA
AQPFLLRNGANEGFHEAVGEIMSLSAATPKHLKSIGLLSPDFQEDNETEINFLLKQALTIVGTLP
FTYMLEKWRWMVFKGEIPKDQWMKKWWEMKREIVGVVEPVPHDETYCDPASLFHVSNDYS
FIRYYTRTLYQFQFQEALCQAAKHEGPLHKCDISNSTEAGQKLFNMLRLGKSEPWTLALENVV
GAKNMNVRPLLNYFEPLFTWLKDQNKNSFVGWSTDWSPYADGGGGSGGGGSGGGGSGGGG
SGGGGS
ACE2-AH5-Foldon DNA Sequence
(SEQ ID NO: 14)
CAGTCCACCATTGAGGAACAGGCCAAGACATTTTTGGACAAGTTTAACCACGAAGCCGAA
GACCTGTTCTATCAAAGTTCACTTGCTTCTTGGAATTATAACACCAATATTACTGAAGAGA
ATGTCCAAAACATGAATAATGCTGGGGACAAATGGTCTGCCTTTTTAAAGGAACAGTCCA
CACTTGCCCAAATGTATCCACTACAAGAAATTCAGAATCTCACAGTCAAGCTTCAGCTGCA
GGCTCTTCAGCAAAATGGGTCTTCAGTGCTCTCAGAAGACAAGAGCAAACGGTTGAACAC
AATTCTAAATACAATGAGCACCATCTACAGTACTGGAAAAGTTTGTAACCCAGATAATCC
ACAAGAATGCTTATTACTTGAACCAGGTTTGAATGAAATAATGGCAAACAGTTTAGACTA
CAATGAGAGGCTCTGGGCTTGGGAAAGCTGGAGATCTGAGGTCGGCAAGCAGCTGAGGC
CATTATATGAAGAGTATGTGGTCTTGAAAAATGAGATGGCAAGAGCAAATCATTATGAGG
ACTATGGGGATTATTGGAGAGGAGACTATGAAGTAAATGGGGTAGATGGCTATGACTACA
GCCGCGGCCAGTTGATTGAAGATGTGGAACATACCTTTGAAGAGATTAAACCATTATATG
AACATCTTCATGCCTATGTGAGGGCAAAGTTGATGAATGCCTATCCTTCCTATATCAGTCC
AATTGGATGCCTCCCTGCTCATTTGCTTGGTGATATGTGGGGTAGATTTTGGACAAATCTG
TACTCTTTGACAGTTCCCTTTGGACAGAAACCAAACATAGATGTTACTGATGCAATGGTGG
ACCAGGCCTGGGATGCACAGAGAATATTCAAGGAGGCCGAGAAGTTCTTTGTATCTGTTG
GTCTTCCTAATATGACTCAAGGATTCTGGGAAAATTCCATGCTAACGGACCCAGGAAATG
TTCAGAAAGCAGTCTGCCATCCCACAGCTTGGGACCTGGGGAAGGGCGACTTCAGGATCC
TTATGTGCACAAAGGTGACAATGGACGACTTCCTGACAGCTCATCATGAGATGGGGCATA
TCCAGTATGATATGGCATATGCTGCACAACCTTTTCTGCTAAGAAATGGAGCTAATGAAG
GATTCCATGAAGCTGTTGGGGAAATCATGTCACTTTCTGCAGCCACACCTAAGCATTTAAA
ATCCATTGGTCTTCTGTCACCCGATTTTCAAGAAGACAATGAAACAGAAATAAACTTCCTG
CTCAAACAAGCACTCACGATTGTTGGGACTCTGCCATTTACTTACATGTTAGAGAAGTGGA
GGTGGATGGTCTTTAAAGGGGAAATTCCCAAAGACCAGTGGATGAAAAAGTGGTGGGAG
ATGAAGCGAGAGATAGTTGGGGTGGTGGAACCTGTGCCCCATGATGAAACATACTGTGAC
CCCGCATCTCTGTTCCATGTTTCTAATGATTACTCATTCATTCGATATTACACAAGGACCCT
TTACCAATTCCAGTTTCAAGAAGCACTTTGTCAAGCAGCTAAACATGAAGGCCCTCTGCAC
AAATGTGACATCTCAAACTCTACAGAAGCTGGACAGAAACTGTTCAATATGCTGAGGCTT
GGAAAATCAGAACCCTGGACCCTAGCATTGGAAAATGTTGTAGGAGCAAAGAACATGAA
TGTAAGGCCACTGCTCAACTACTTTGAGCCCTTATTTACCTGGCTGAAAGACCAGAACAAG
AATTCTTTTGTGGGATGGAGTACCGACTGGAGTCCATATGCAGACGGCAGCGCCGAGGCC
GCCGCCAAGGAAGCCGCTGCTAAGGAAGCCGCTGCCAAGGAGGCCGCCGCCAAAGAGGC
CGCCGCTAAGGCCGGCTCTGGCTACATCCCCGAGGCCCCCAGGGACGGCCAGGCCTACGT
GAGGAAGGACGGCGAGTGGGTGCTGCTGAGCACCTTCCTG
ACE2-AH5-Foldon Protein Sequence
(SEQ ID NO: 15)
QSTIEEQAKTFLDKFNHEAEDLFYQSSLASWNYNTNITEENVQNMNNAGDKWSAFLKEQSTL
AQMYPLQEIQNLTVKLQLQALQQNGSSVLSEDKSKRLNTILNTMSTIYSTGKVCNPDNPQECL
LLEPGLNEIMANSLDYNERLWAWESWRSEVGKQLRPLYEEYVVLKNEMARANHYEDYGDY
WRGDYEVNGVDGYDYSRGQLIEDVEHTFEEIKPLYEHLHAYVRAKLMNAYPSYISPIGCLPAH
LLGDMWGRFWTNLYSLTVPFGQKPNIDVTDAMVDQAWDAQRIFKEAEKFFVSVGLPNMTQG
FWENSMLTDPGNVQKAVCHPTAWDLGKGDFRILMCTKVTMDDFLTAHHEMGHIQYDMAYA
AQPFLLRNGANEGFHEAVGEIMSLSAATPKHLKSIGLLSPDFQEDNETEINFLLKQALTIVGTLP
FTYMLEKWRWMVFKGEIPKDQWMKKWWEMKREIVGVVEPVPHDETYCDPASLFHVSNDYS
FIRYYTRTLYQFQFQEALCQAAKHEGPLHKCDISNSTEAGQKLFNMLRLGKSEPWTLALENVV
GAKNMNVRPLLNYFEPLFTWLKDQNKNSFVGWSTDWSPYADGSAEAAAKEAAAKEAAAKE
AAAKEAAAKAGSYIPEAPRDGQAYVRKDGEWVLLSTFL
ACE2-GGGH5-Foldon DNA Sequence
(SEQ ID NO: 16)
CAGTCCACCATTGAGGAACAGGCCAAGACATTTTTGGACAAGTTTAACCACGAAGCCGAA
GACCTGTTCTATCAAAGTTCACTTGCTTCTTGGAATTATAACACCAATATTACTGAAGAGA
ATGTCCAAAACATGAATAATGCTGGGGACAAATGGTCTGCCTTTTTAAAGGAACAGTCCA
CACTTGCCCAAATGTATCCACTACAAGAAATTCAGAATCTCACAGTCAAGCTTCAGCTGCA
GGCTCTTCAGCAAAATGGGTCTTCAGTGCTCTCAGAAGACAAGAGCAAACGGTTGAACAC
AATTCTAAATACAATGAGCACCATCTACAGTACTGGAAAAGTTTGTAACCCAGATAATCC
ACAAGAATGCTTATTACTTGAACCAGGTTTGAATGAAATAATGGCAAACAGTTTAGACTA
CAATGAGAGGCTCTGGGCTTGGGAAAGCTGGAGATCTGAGGTCGGCAAGCAGCTGAGGC
CATTATATGAAGAGTATGTGGTCTTGAAAAATGAGATGGCAAGAGCAAATCATTATGAGG
ACTATGGGGATTATTGGAGAGGAGACTATGAAGTAAATGGGGTAGATGGCTATGACTACA
GCCGCGGCCAGTTGATTGAAGATGTGGAACATACCTTTGAAGAGATTAAACCATTATATG
AACATCTTCATGCCTATGTGAGGGCAAAGTTGATGAATGCCTATCCTTCCTATATCAGTCC
AATTGGATGCCTCCCTGCTCATTTGCTTGGTGATATGTGGGGTAGATTTTGGACAAATCTG
TACTCTTTGACAGTTCCCTTTGGACAGAAACCAAACATAGATGTTACTGATGCAATGGTGG
ACCAGGCCTGGGATGCACAGAGAATATTCAAGGAGGCCGAGAAGTTCTTTGTATCTGTTG
GTCTTCCTAATATGACTCAAGGATTCTGGGAAAATTCCATGCTAACGGACCCAGGAAATG
TTCAGAAAGCAGTCTGCCATCCCACAGCTTGGGACCTGGGGAAGGGCGACTTCAGGATCC
TTATGTGCACAAAGGTGACAATGGACGACTTCCTGACAGCTCATCATGAGATGGGGCATA
TCCAGTATGATATGGCATATGCTGCACAACCTTTTCTGCTAAGAAATGGAGCTAATGAAG
GATTCCATGAAGCTGTTGGGGAAATCATGTCACTTTCTGCAGCCACACCTAAGCATTTAAA
ATCCATTGGTCTTCTGTCACCCGATTTTCAAGAAGACAATGAAACAGAAATAAACTTCCTG
CTCAAACAAGCACTCACGATTGTTGGGACTCTGCCATTTACTTACATGTTAGAGAAGTGGA
GGTGGATGGTCTTTAAAGGGGAAATTCCCAAAGACCAGTGGATGAAAAAGTGGTGGGAG
ATGAAGCGAGAGATAGTTGGGGTGGTGGAACCTGTGCCCCATGATGAAACATACTGTGAC
CCCGCATCTCTGTTCCATGTTTCTAATGATTACTCATTCATTCGATATTACACAAGGACCCT
TTACCAATTCCAGTTTCAAGAAGCACTTTGTCAAGCAGCTAAACATGAAGGCCCTCTGCAC
AAATGTGACATCTCAAACTCTACAGAAGCTGGACAGAAACTGTTCAATATGCTGAGGCTT
GGAAAATCAGAACCCTGGACCCTAGCATTGGAAAATGTTGTAGGAGCAAAGAACATGAA
TGTAAGGCCACTGCTCAACTACTTTGAGCCCTTATTTACCTGGCTGAAAGACCAGAACAAG
AATTCTTTTGTGGGATGGAGTACCGACTGGAGTCCATATGCAGACGGCGGCGGCGGCAGC
GAGGCCGCCGCCAAGGAAGCCGCTGCCAAGGAAGCCGCCGCTAAAGAGGCCGCTGCCAA
GGAAGCCGCCGCTAAGGGCGGAGGCGGATCTGGCTACATCCCCGAGGCCCCCAGGGACG
GCCAGGCCTACGTGAGGAAGGACGGCGAGTGGGTGCTGCTGAGCACCTTCCTG
ACE2-GGGH5-Foldon Protein Sequence
(SEQ ID NO: 17)
QSTIEEQAKTFLDKFNHEAEDLFYQSSLASWNYNTNITEENVQNMNNAGDKWSAFLKEQSTL
AQMYPLQEIQNLTVKLQLQALQQNGSSVLSEDKSKRLNTILNTMSTIYSTGKVCNPDNPQECL
LLEPGLNEIMANSLDYNERLWAWESWRSEVGKQLRPLYEEYVVLKNEMARANHYEDYGDY
WRGDYEVNGVDGYDYSRGQLIEDVEHTFEEIKPLYEHLHAYVRAKLMNAYPSYISPIGCLPAH
LLGDMWGRFWTNLYSLTVPFGQKPNIDVTDAMVDQAWDAQRIFKEAEKFFVSVGLPNMTQG
FWENSMLTDPGNVQKAVCHPTAWDLGKGDFRILMCTKVTMDDFLTAHHEMGHIQYDMAYA
AQPFLLRNGANEGFHEAVGEIMSLSAATPKHLKSIGLLSPDFQEDNETEINFLLKQALTIVGTLP
FTYMLEKWRWMVFKGEIPKDQWMKKWWEMKREIVGVVEPVPHDETYCDPASLFHVSNDYS
FIRYYTRTLYQFQFQEALCQAAKHEGPLHKCDISNSTEAGQKLFNMLRLGKSEPWTLALENVV
GAKNMNVRPLLNYFEPLFTWLKDQNKNSFVGWSTDWSPYADGGGGSEAAAKEAAAKEAAA
KEAAAKEAAAKGGGGSGYIPEAPRDGQAYVRKDGEWVLLSTFL
ACE2-H3-Foldon DNA Sequence
(SEQ ID NO: 18)
CAGTCCACCATTGAGGAACAGGCCAAGACATTTTTGGACAAGTTTAACCACGAAGCCGAA
GACCTGTTCTATCAAAGTTCACTTGCTTCTTGGAATTATAACACCAATATTACTGAAGAGA
ATGTCCAAAACATGAATAATGCTGGGGACAAATGGTCTGCCTTTTTAAAGGAACAGTCCA
CACTTGCCCAAATGTATCCACTACAAGAAATTCAGAATCTCACAGTCAAGCTTCAGCTGCA
GGCTCTTCAGCAAAATGGGTCTTCAGTGCTCTCAGAAGACAAGAGCAAACGGTTGAACAC
AATTCTAAATACAATGAGCACCATCTACAGTACTGGAAAAGTTTGTAACCCAGATAATCC
ACAAGAATGCTTATTACTTGAACCAGGTTTGAATGAAATAATGGCAAACAGTTTAGACTA
CAATGAGAGGCTCTGGGCTTGGGAAAGCTGGAGATCTGAGGTCGGCAAGCAGCTGAGGC
CATTATATGAAGAGTATGTGGTCTTGAAAAATGAGATGGCAAGAGCAAATCATTATGAGG
ACTATGGGGATTATTGGAGAGGAGACTATGAAGTAAATGGGGTAGATGGCTATGACTACA
GCCGCGGCCAGTTGATTGAAGATGTGGAACATACCTTTGAAGAGATTAAACCATTATATG
AACATCTTCATGCCTATGTGAGGGCAAAGTTGATGAATGCCTATCCTTCCTATATCAGTCC
AATTGGATGCCTCCCTGCTCATTTGCTTGGTGATATGTGGGGTAGATTTTGGACAAATCTG
TACTCTTTGACAGTTCCCTTTGGACAGAAACCAAACATAGATGTTACTGATGCAATGGTGG
ACCAGGCCTGGGATGCACAGAGAATATTCAAGGAGGCCGAGAAGTTCTTTGTATCTGTTG
GTCTTCCTAATATGACTCAAGGATTCTGGGAAAATTCCATGCTAACGGACCCAGGAAATG
TTCAGAAAGCAGTCTGCCATCCCACAGCTTGGGACCTGGGGAAGGGCGACTTCAGGATCC
TTATGTGCACAAAGGTGACAATGGACGACTTCCTGACAGCTCATCATGAGATGGGGCATA
TCCAGTATGATATGGCATATGCTGCACAACCTTTTCTGCTAAGAAATGGAGCTAATGAAG
GATTCCATGAAGCTGTTGGGGAAATCATGTCACTTTCTGCAGCCACACCTAAGCATTTAAA
ATCCATTGGTCTTCTGTCACCCGATTTTCAAGAAGACAATGAAACAGAAATAAACTTCCTG
CTCAAACAAGCACTCACGATTGTTGGGACTCTGCCATTTACTTACATGTTAGAGAAGTGGA
GGTGGATGGTCTTTAAAGGGGAAATTCCCAAAGACCAGTGGATGAAAAAGTGGTGGGAG
ATGAAGCGAGAGATAGTTGGGGTGGTGGAACCTGTGCCCCATGATGAAACATACTGTGAC
CCCGCATCTCTGTTCCATGTTTCTAATGATTACTCATTCATTCGATATTACACAAGGACCCT
TTACCAATTCCAGTTTCAAGAAGCACTTTGTCAAGCAGCTAAACATGAAGGCCCTCTGCAC
AAATGTGACATCTCAAACTCTACAGAAGCTGGACAGAAACTGTTCAATATGCTGAGGCTT
GGAAAATCAGAACCCTGGACCCTAGCATTGGAAAATGTTGTAGGAGCAAAGAACATGAA
TGTAAGGCCACTGCTCAACTACTTTGAGCCCTTATTTACCTGGCTGAAAGACCAGAACAAG
AATTCTTTTGTGGGATGGAGTACCGACTGGAGTCCATATGCAGACGGCAGCGAGGCCGCC
GCTAAAGAGGCCGCCGCCAAGGAAGCCGCTGCCAAGGGCTCTGGCTACATCCCCGAGGCC
CCCAGGGACGGCCAGGCCTACGTGAGGAAGGACGGCGAGTGGGTGCTGCTGAGCACCTTC
CTG
ACE2-H3-Foldon Protein Sequence
(SEQ ID NO: 19)
QSTIEEQAKTFLDKFNHEAEDLFYQSSLASWNYNTNITEENVQNMNNAGDKWSAFLKEQSTL
AQMYPLQEIQNLTVKLQLQALQQNGSSVLSEDKSKRLNTILNTMSTIYSTGKVCNPDNPQECL
LLEPGLNEIMANSLDYNERLWAWESWRSEVGKQLRPLYEEYVVLKNEMARANHYEDYGDY
WRGDYEVNGVDGYDYSRGQLIEDVEHTFEEIKPLYEHLHAYVRAKLMNAYPSYISPIGCLPAH
LLGDMWGRFWTNLYSLTVPFGQKPNIDVTDAMVDQAWDAQRIFKEAEKFFVSVGLPNMTQG
FWENSMLTDPGNVQKAVCHPTAWDLGKGDFRILMCTKVTMDDFLTAHHEMGHIQYDMAYA
AQPFLLRNGANEGFHEAVGEIMSLSAATPKHLKSIGLLSPDFQEDNETEINFLLKQALTIVGTLP
FTYMLEKWRWMVFKGEIPKDQWMKKWWEMKREIVGVVEPVPHDETYCDPASLFHVSNDYS
FIRYYTRTLYQFQFQEALCQAAKHEGPLHKCDISNSTEAGQKLFNMLRLGKSEPWTLALENVV
GAKNMNVRPLLNYFEPLFTWLKDQNKNSFVGWSTDWSPYADGSEAAAKEAAAKEAAAKGS
YIPEAPRDGQAYVRKDGEWVLLSTFL
ACE2-H4-Foldon DNA Sequence
(SEQ ID NO: 20)
CAGTCCACCATTGAGGAACAGGCCAAGACATTTTTGGACAAGTTTAACCACGAAGCCGAA
GACCTGTTCTATCAAAGTTCACTTGCTTCTTGGAATTATAACACCAATATTACTGAAGAGA
ATGTCCAAAACATGAATAATGCTGGGGACAAATGGTCTGCCTTTTTAAAGGAACAGTCCA
CACTTGCCCAAATGTATCCACTACAAGAAATTCAGAATCTCACAGTCAAGCTTCAGCTGCA
GGCTCTTCAGCAAAATGGGTCTTCAGTGCTCTCAGAAGACAAGAGCAAACGGTTGAACAC
AATTCTAAATACAATGAGCACCATCTACAGTACTGGAAAAGTTTGTAACCCAGATAATCC
ACAAGAATGCTTATTACTTGAACCAGGTTTGAATGAAATAATGGCAAACAGTTTAGACTA
CAATGAGAGGCTCTGGGCTTGGGAAAGCTGGAGATCTGAGGTCGGCAAGCAGCTGAGGC
CATTATATGAAGAGTATGTGGTCTTGAAAAATGAGATGGCAAGAGCAAATCATTATGAGG
ACTATGGGGATTATTGGAGAGGAGACTATGAAGTAAATGGGGTAGATGGCTATGACTACA
GCCGCGGCCAGTTGATTGAAGATGTGGAACATACCTTTGAAGAGATTAAACCATTATATG
AACATCTTCATGCCTATGTGAGGGCAAAGTTGATGAATGCCTATCCTTCCTATATCAGTCC
AATTGGATGCCTCCCTGCTCATTTGCTTGGTGATATGTGGGGTAGATTTTGGACAAATCTG
TACTCTTTGACAGTTCCCTTTGGACAGAAACCAAACATAGATGTTACTGATGCAATGGTGG
ACCAGGCCTGGGATGCACAGAGAATATTCAAGGAGGCCGAGAAGTTCTTTGTATCTGTTG
GTCTTCCTAATATGACTCAAGGATTCTGGGAAAATTCCATGCTAACGGACCCAGGAAATG
TTCAGAAAGCAGTCTGCCATCCCACAGCTTGGGACCTGGGGAAGGGCGACTTCAGGATCC
TTATGTGCACAAAGGTGACAATGGACGACTTCCTGACAGCTCATCATGAGATGGGGCATA
TCCAGTATGATATGGCATATGCTGCACAACCTTTTCTGCTAAGAAATGGAGCTAATGAAG
GATTCCATGAAGCTGTTGGGGAAATCATGTCACTTTCTGCAGCCACACCTAAGCATTTAAA
ATCCATTGGTCTTCTGTCACCCGATTTTCAAGAAGACAATGAAACAGAAATAAACTTCCTG
CTCAAACAAGCACTCACGATTGTTGGGACTCTGCCATTTACTTACATGTTAGAGAAGTGGA
GGTGGATGGTCTTTAAAGGGGAAATTCCCAAAGACCAGTGGATGAAAAAGTGGTGGGAG
ATGAAGCGAGAGATAGTTGGGGTGGTGGAACCTGTGCCCCATGATGAAACATACTGTGAC
CCCGCATCTCTGTTCCATGTTTCTAATGATTACTCATTCATTCGATATTACACAAGGACCCT
TTACCAATTCCAGTTTCAAGAAGCACTTTGTCAAGCAGCTAAACATGAAGGCCCTCTGCAC
AAATGTGACATCTCAAACTCTACAGAAGCTGGACAGAAACTGTTCAATATGCTGAGGCTT
GGAAAATCAGAACCCTGGACCCTAGCATTGGAAAATGTTGTAGGAGCAAAGAACATGAA
TGTAAGGCCACTGCTCAACTACTTTGAGCCCTTATTTACCTGGCTGAAAGACCAGAACAAG
AATTCTTTTGTGGGATGGAGTACCGACTGGAGTCCATATGCAGACGGCAGCGAGGCCGCC
GCCAAGGAAGCCGCCGCCAAGGAAGCCGCTGCTAAAGAGGCCGCTGCCAAGGGCTCTGG
CTACATCCCCGAGGCCCCCAGGGACGGCCAGGCCTACGTGAGGAAGGACGGCGAGTGGG
TGCTGCTGAGCACCTTCCTG
ACE2-H4-Foldon Protein Sequence
(SEQ ID NO: 21)
QSTIEEQAKTFLDKFNHEAEDLFYQSSLASWNYNTNITEENVQNMNNAGDKWSAFLKEQSTL
AQMYPLQEIQNLTVKLQLQALQQNGSSVLSEDKSKRLNTILNTMSTIYSTGKVCNPDNPQECL
LLEPGLNEIMANSLDYNERLWAWESWRSEVGKQLRPLYEEYVVLKNEMARANHYEDYGDY
WRGDYEVNGVDGYDYSRGQLIEDVEHTFEEIKPLYEHLHAYVRAKLMNAYPSYISPIGCLPAH
LLGDMWGRFWTNLYSLTVPFGQKPNIDVTDAMVDQAWDAQRIFKEAEKFFVSVGLPNMTQG
FWENSMLTDPGNVQKAVCHPTAWDLGKGDFRILMCTKVTMDDFLTAHHEMGHIQYDMAYA
AQPFLLRNGANEGFHEAVGEIMSLSAATPKHLKSIGLLSPDFQEDNETEINFLLKQALTIVGTLP
FTYMLEKWRWMVFKGEIPKDQWMKKWWEMKREIVGVVEPVPHDETYCDPASLFHVSNDYS
FIRYYTRTLYQFQFQEALCQAAKHEGPLHKCDISNSTEAGQKLFNMLRLGKSEPWTLALENVV
GAKNMNVRPLLNYFEPLFTWLKDQNKNSFVGWSTDWSPYADGSEAAAKEAAAKEAAAKEA
AAKGSYIPEAPRDGQAYVRKDGEWVLLSTFL
ACE2-H6-Foldon DNA Sequence
(SEQ ID NO: 22)
CAGTCCACCATTGAGGAACAGGCCAAGACATTTTTGGACAAGTTTAACCACGAAGCCGAA
GACCTGTTCTATCAAAGTTCACTTGCTTCTTGGAATTATAACACCAATATTACTGAAGAGA
ATGTCCAAAACATGAATAATGCTGGGGACAAATGGTCTGCCTTTTTAAAGGAACAGTCCA
CACTTGCCCAAATGTATCCACTACAAGAAATTCAGAATCTCACAGTCAAGCTTCAGCTGCA
GGCTCTTCAGCAAAATGGGTCTTCAGTGCTCTCAGAAGACAAGAGCAAACGGTTGAACAC
AATTCTAAATACAATGAGCACCATCTACAGTACTGGAAAAGTTTGTAACCCAGATAATCC
ACAAGAATGCTTATTACTTGAACCAGGTTTGAATGAAATAATGGCAAACAGTTTAGACTA
CAATGAGAGGCTCTGGGCTTGGGAAAGCTGGAGATCTGAGGTCGGCAAGCAGCTGAGGC
CATTATATGAAGAGTATGTGGTCTTGAAAAATGAGATGGCAAGAGCAAATCATTATGAGG
ACTATGGGGATTATTGGAGAGGAGACTATGAAGTAAATGGGGTAGATGGCTATGACTACA
GCCGCGGCCAGTTGATTGAAGATGTGGAACATACCTTTGAAGAGATTAAACCATTATATG
AACATCTTCATGCCTATGTGAGGGCAAAGTTGATGAATGCCTATCCTTCCTATATCAGTCC
AATTGGATGCCTCCCTGCTCATTTGCTTGGTGATATGTGGGGTAGATTTTGGACAAATCTG
TACTCTTTGACAGTTCCCTTTGGACAGAAACCAAACATAGATGTTACTGATGCAATGGTGG
ACCAGGCCTGGGATGCACAGAGAATATTCAAGGAGGCCGAGAAGTTCTTTGTATCTGTTG
GTCTTCCTAATATGACTCAAGGATTCTGGGAAAATTCCATGCTAACGGACCCAGGAAATG
TTCAGAAAGCAGTCTGCCATCCCACAGCTTGGGACCTGGGGAAGGGCGACTTCAGGATCC
TTATGTGCACAAAGGTGACAATGGACGACTTCCTGACAGCTCATCATGAGATGGGGCATA
TCCAGTATGATATGGCATATGCTGCACAACCTTTTCTGCTAAGAAATGGAGCTAATGAAG
GATTCCATGAAGCTGTTGGGGAAATCATGTCACTTTCTGCAGCCACACCTAAGCATTTAAA
ATCCATTGGTCTTCTGTCACCCGATTTTCAAGAAGACAATGAAACAGAAATAAACTTCCTG
CTCAAACAAGCACTCACGATTGTTGGGACTCTGCCATTTACTTACATGTTAGAGAAGTGGA
GGTGGATGGTCTTTAAAGGGGAAATTCCCAAAGACCAGTGGATGAAAAAGTGGTGGGAG
ATGAAGCGAGAGATAGTTGGGGTGGTGGAACCTGTGCCCCATGATGAAACATACTGTGAC
CCCGCATCTCTGTTCCATGTTTCTAATGATTACTCATTCATTCGATATTACACAAGGACCCT
TTACCAATTCCAGTTTCAAGAAGCACTTTGTCAAGCAGCTAAACATGAAGGCCCTCTGCAC
AAATGTGACATCTCAAACTCTACAGAAGCTGGACAGAAACTGTTCAATATGCTGAGGCTT
GGAAAATCAGAACCCTGGACCCTAGCATTGGAAAATGTTGTAGGAGCAAAGAACATGAA
TGTAAGGCCACTGCTCAACTACTTTGAGCCCTTATTTACCTGGCTGAAAGACCAGAACAAG
AATTCTTTTGTGGGATGGAGTACCGACTGGAGTCCATATGCAGACGGATCTGAGGCCGCT
GCAAAGGAAGCCGCCGCTAAAGAGGCCGCCGCTAAGGAAGCCGCTGCCAAGGAAGCCGC
CGCCAAAGAGGCCGCCGCCAAGGGCAGCGGCTACATCCCCGAGGCCCCCAGGGACGGCC
AGGCCTACGTGAGGAAGGACGGCGAGTGGGTGCTGCTGAGCACCTTCCTG
ACE2-H6-Foldon Protein Sequence
(SEQ ID NO: 23)
QSTIEEQAKTFLDKFNHEAEDLFYQSSLASWNYNTNITEENVQNMNNAGDKWSAFLKEQSTL
AQMYPLQEIQNLTVKLQLQALQQNGSSVLSEDKSKRLNTILNTMSTIYSTGKVCNPDNPQECL
LLEPGLNEIMANSLDYNERLWAWESWRSEVGKQLRPLYEEYVVLKNEMARANHYEDYGDY
WRGDYEVNGVDGYDYSRGQLIEDVEHTFEEIKPLYEHLHAYVRAKLMNAYPSYISPIGCLPAH
LLGDMWGRFWTNLYSLTVPFGQKPNIDVTDAMVDQAWDAQRIFKEAEKFFVSVGLPNMTQG
FWENSMLTDPGNVQKAVCHPTAWDLGKGDFRILMCTKVTMDDFLTAHHEMGHIQYDMAYA
AQPFLLRNGANEGFHEAVGEIMSLSAATPKHLKSIGLLSPDFQEDNETEINFLLKQALTIVGTLP
FTYMLEKWRWMVFKGEIPKDQWMKKWWEMKREIVGVVEPVPHDETYCDPASLFHVSNDYS
FIRYYTRTLYQFQFQEALCQAAKHEGPLHKCDISNSTEAGQKLFNMLRLGKSEPWTLALENVV
GAKNMNVRPLLNYFEPLFTWLKDQNKNSFVGWSTDWSPYADGSEAAAKEAAAKEAAAKEA
AAKEAAAKEAAAKGSGYIPEAPRDGQAYVRKDGEWVLLSTFL
ACE2-H7-Foldon DNA Sequence
(SEQ ID NO: 24)
CAGTCCACCATTGAGGAACAGGCCAAGACATTTTTGGACAAGTTTAACCACGAAGCCGAA
GACCTGTTCTATCAAAGTTCACTTGCTTCTTGGAATTATAACACCAATATTACTGAAGAGA
ATGTCCAAAACATGAATAATGCTGGGGACAAATGGTCTGCCTTTTTAAAGGAACAGTCCA
CACTTGCCCAAATGTATCCACTACAAGAAATTCAGAATCTCACAGTCAAGCTTCAGCTGCA
GGCTCTTCAGCAAAATGGGTCTTCAGTGCTCTCAGAAGACAAGAGCAAACGGTTGAACAC
AATTCTAAATACAATGAGCACCATCTACAGTACTGGAAAAGTTTGTAACCCAGATAATCC
ACAAGAATGCTTATTACTTGAACCAGGTTTGAATGAAATAATGGCAAACAGTTTAGACTA
CAATGAGAGGCTCTGGGCTTGGGAAAGCTGGAGATCTGAGGTCGGCAAGCAGCTGAGGC
CATTATATGAAGAGTATGTGGTCTTGAAAAATGAGATGGCAAGAGCAAATCATTATGAGG
ACTATGGGGATTATTGGAGAGGAGACTATGAAGTAAATGGGGTAGATGGCTATGACTACA
GCCGCGGCCAGTTGATTGAAGATGTGGAACATACCTTTGAAGAGATTAAACCATTATATG
AACATCTTCATGCCTATGTGAGGGCAAAGTTGATGAATGCCTATCCTTCCTATATCAGTCC
AATTGGATGCCTCCCTGCTCATTTGCTTGGTGATATGTGGGGTAGATTTTGGACAAATCTG
TACTCTTTGACAGTTCCCTTTGGACAGAAACCAAACATAGATGTTACTGATGCAATGGTGG
ACCAGGCCTGGGATGCACAGAGAATATTCAAGGAGGCCGAGAAGTTCTTTGTATCTGTTG
GTCTTCCTAATATGACTCAAGGATTCTGGGAAAATTCCATGCTAACGGACCCAGGAAATG
TTCAGAAAGCAGTCTGCCATCCCACAGCTTGGGACCTGGGGAAGGGCGACTTCAGGATCC
TTATGTGCACAAAGGTGACAATGGACGACTTCCTGACAGCTCATCATGAGATGGGGCATA
TCCAGTATGATATGGCATATGCTGCACAACCTTTTCTGCTAAGAAATGGAGCTAATGAAG
GATTCCATGAAGCTGTTGGGGAAATCATGTCACTTTCTGCAGCCACACCTAAGCATTTAAA
ATCCATTGGTCTTCTGTCACCCGATTTTCAAGAAGACAATGAAACAGAAATAAACTTCCTG
CTCAAACAAGCACTCACGATTGTTGGGACTCTGCCATTTACTTACATGTTAGAGAAGTGGA
GGTGGATGGTCTTTAAAGGGGAAATTCCCAAAGACCAGTGGATGAAAAAGTGGTGGGAG
ATGAAGCGAGAGATAGTTGGGGTGGTGGAACCTGTGCCCCATGATGAAACATACTGTGAC
CCCGCATCTCTGTTCCATGTTTCTAATGATTACTCATTCATTCGATATTACACAAGGACCCT
TTACCAATTCCAGTTTCAAGAAGCACTTTGTCAAGCAGCTAAACATGAAGGCCCTCTGCAC
AAATGTGACATCTCAAACTCTACAGAAGCTGGACAGAAACTGTTCAATATGCTGAGGCTT
GGAAAATCAGAACCCTGGACCCTAGCATTGGAAAATGTTGTAGGAGCAAAGAACATGAA
TGTAAGGCCACTGCTCAACTACTTTGAGCCCTTATTTACCTGGCTGAAAGACCAGAACAAG
AATTCTTTTGTGGGATGGAGTACCGACTGGAGTCCATATGCAGACGGCAGCGAGGCCGCT
GCCAAAGAGGCCGCCGCCAAGGAAGCCGCTGCAAAGGAAGCCGCCGCTAAAGAGGCCGC
TGCCAAGGAGGCCGCCGCCAAGGAAGCCGCCGCCAAGGGCTCTGGCTACATCCCCGAGGC
CCCCAGGGACGGCCAGGCCTACGTGAGGAAGGACGGCGAGTGGGTGCTGCTGAGCACCTT
CCTG
ACE2-H7-Foldon Protein Sequence
(SEQ ID NO: 25)
QSTIEEQAKTFLDKFNHEAEDLFYQSSLASWNYNTNITEENVQNMNNAGDKWSAFLKEQSTL
AQMYPLQEIQNLTVKLQLQALQQNGSSVLSEDKSKRLNTILNTMSTIYSTGKVCNPDNPQECL
LLEPGLNEIMANSLDYNERLWAWESWRSEVGKQLRPLYEEYVVLKNEMARANHYEDYGDY
WRGDYEVNGVDGYDYSRGQLIEDVEHTFEEIKPLYEHLHAYVRAKLMNAYPSYISPIGCLPAH
LLGDMWGRFWTNLYSLTVPFGQKPNIDVTDAMVDQAWDAQRIFKEAEKFFVSVGLPNMTQG
FWENSMLTDPGNVQKAVCHPTAWDLGKGDFRILMCTKVTMDDFLTAHHEMGHIQYDMAYA
AQPFLLRNGANEGFHEAVGEIMSLSAATPKHLKSIGLLSPDFQEDNETEINFLLKQALTIVGTLP
FTYMLEKWRWMVFKGEIPKDQWMKKWWEMKREIVGVVEPVPHDETYCDPASLFHVSNDYS
FIRYYTRTLYQFQFQEALCQAAKHEGPLHKCDISNSTEAGQKLFNMLRLGKSEPWTLALENVV
GAKNMNVRPLLNYFEPLFTWLKDQNKNSFVGWSTDWSPYADGSEAAAKEAAAKEAAAKEA
AAKEAAAKEAAAKEAAAKGSYIPEAPRDGQAYVRKDGEWVLLSTFL
ACE2-AP12-Foldon DNA Sequence
(SEQ ID NO: 26)
CAGTCCACCATTGAGGAACAGGCCAAGACATTTTTGGACAAGTTTAACCACGAAGCCGAA
GACCTGTTCTATCAAAGTTCACTTGCTTCTTGGAATTATAACACCAATATTACTGAAGAGA
ATGTCCAAAACATGAATAATGCTGGGGACAAATGGTCTGCCTTTTTAAAGGAACAGTCCA
CACTTGCCCAAATGTATCCACTACAAGAAATTCAGAATCTCACAGTCAAGCTTCAGCTGCA
GGCTCTTCAGCAAAATGGGTCTTCAGTGCTCTCAGAAGACAAGAGCAAACGGTTGAACAC
AATTCTAAATACAATGAGCACCATCTACAGTACTGGAAAAGTTTGTAACCCAGATAATCC
ACAAGAATGCTTATTACTTGAACCAGGTTTGAATGAAATAATGGCAAACAGTTTAGACTA
CAATGAGAGGCTCTGGGCTTGGGAAAGCTGGAGATCTGAGGTCGGCAAGCAGCTGAGGC
CATTATATGAAGAGTATGTGGTCTTGAAAAATGAGATGGCAAGAGCAAATCATTATGAGG
ACTATGGGGATTATTGGAGAGGAGACTATGAAGTAAATGGGGTAGATGGCTATGACTACA
GCCGCGGCCAGTTGATTGAAGATGTGGAACATACCTTTGAAGAGATTAAACCATTATATG
AACATCTTCATGCCTATGTGAGGGCAAAGTTGATGAATGCCTATCCTTCCTATATCAGTCC
AATTGGATGCCTCCCTGCTCATTTGCTTGGTGATATGTGGGGTAGATTTTGGACAAATCTG
TACTCTTTGACAGTTCCCTTTGGACAGAAACCAAACATAGATGTTACTGATGCAATGGTGG
ACCAGGCCTGGGATGCACAGAGAATATTCAAGGAGGCCGAGAAGTTCTTTGTATCTGTTG
GTCTTCCTAATATGACTCAAGGATTCTGGGAAAATTCCATGCTAACGGACCCAGGAAATG
TTCAGAAAGCAGTCTGCCATCCCACAGCTTGGGACCTGGGGAAGGGCGACTTCAGGATCC
TTATGTGCACAAAGGTGACAATGGACGACTTCCTGACAGCTCATCATGAGATGGGGCATA
TCCAGTATGATATGGCATATGCTGCACAACCTTTTCTGCTAAGAAATGGAGCTAATGAAG
GATTCCATGAAGCTGTTGGGGAAATCATGTCACTTTCTGCAGCCACACCTAAGCATTTAAA
ATCCATTGGTCTTCTGTCACCCGATTTTCAAGAAGACAATGAAACAGAAATAAACTTCCTG
CTCAAACAAGCACTCACGATTGTTGGGACTCTGCCATTTACTTACATGTTAGAGAAGTGGA
GGTGGATGGTCTTTAAAGGGGAAATTCCCAAAGACCAGTGGATGAAAAAGTGGTGGGAG
ATGAAGCGAGAGATAGTTGGGGTGGTGGAACCTGTGCCCCATGATGAAACATACTGTGAC
CCCGCATCTCTGTTCCATGTTTCTAATGATTACTCATTCATTCGATATTACACAAGGACCCT
TTACCAATTCCAGTTTCAAGAAGCACTTTGTCAAGCAGCTAAACATGAAGGCCCTCTGCAC
AAATGTGACATCTCAAACTCTACAGAAGCTGGACAGAAACTGTTCAATATGCTGAGGCTT
GGAAAATCAGAACCCTGGACCCTAGCATTGGAAAATGTTGTAGGAGCAAAGAACATGAA
TGTAAGGCCACTGCTCAACTACTTTGAGCCCTTATTTACCTGGCTGAAAGACCAGAACAAG
AATTCTTTTGTGGGATGGAGTACCGACTGGAGTCCATATGCAGACGGATCTGCCCCAGCCC
CTGCCCCTGCCCCTGCTCCAGCTCCCGCCCCTGCCCCTGCCCCTGCCCCTGCCCCTGCTCCC
GGCAGCGGCTACATCCCCGAGGCCCCCAGGGACGGCCAGGCCTACGTGAGGAAGGACGG
CGAGTGGGTGCTGCTGAGCACCTTCCTG
ACE2-AP12-Foldon Protein Sequence
(SEQ ID NO: 27)
QSTIEEQAKTFLDKFNHEAEDLFYQSSLASWNYNTNITEENVQNMNNAGDKWSAFLKEQSTL
AQMYPLQEIQNLTVKLQLQALQQNGSSVLSEDKSKRLNTILNTMSTIYSTGKVCNPDNPQECL
LLEPGLNEIMANSLDYNERLWAWESWRSEVGKQLRPLYEEYVVLKNEMARANHYEDYGDY
WRGDYEVNGVDGYDYSRGQLIEDVEHTFEEIKPLYEHLHAYVRAKLMNAYPSYISPIGCLPAH
LLGDMWGRFWTNLYSLTVPFGQKPNIDVTDAMVDQAWDAQRIFKEAEKFFVSVGLPNMTQG
FWENSMLTDPGNVQKAVCHPTAWDLGKGDFRILMCTKVTMDDFLTAHHEMGHIQYDMAYA
AQPFLLRNGANEGFHEAVGEIMSLSAATPKHLKSIGLLSPDFQEDNETEINFLLKQALTIVGTLP
FTYMLEKWRWMVFKGEIPKDQWMKKWWEMKREIVGVVEPVPHDETYCDPASLFHVSNDYS
FIRYYTRTLYQFQFQEALCQAAKHEGPLHKCDISNSTEAGQKLFNMLRLGKSEPWTLALENVV
GAKNMNVRPLLNYFEPLFTWLKDQNKNSFVGWSTDWSPYADGSAPAPAPAPAPAPAPAPAP
APAPAPGSGYIPEAPRDGQAYVRKDGEWVLLSTFL
ACE2-AP15-Foldon DNA Sequence
(SEQ ID NO: 28)
CAGTCCACCATTGAGGAACAGGCCAAGACATTTTTGGACAAGTTTAACCACGAAGCCGAA
GACCTGTTCTATCAAAGTTCACTTGCTTCTTGGAATTATAACACCAATATTACTGAAGAGA
ATGTCCAAAACATGAATAATGCTGGGGACAAATGGTCTGCCTTTTTAAAGGAACAGTCCA
CACTTGCCCAAATGTATCCACTACAAGAAATTCAGAATCTCACAGTCAAGCTTCAGCTGCA
GGCTCTTCAGCAAAATGGGTCTTCAGTGCTCTCAGAAGACAAGAGCAAACGGTTGAACAC
AATTCTAAATACAATGAGCACCATCTACAGTACTGGAAAAGTTTGTAACCCAGATAATCC
ACAAGAATGCTTATTACTTGAACCAGGTTTGAATGAAATAATGGCAAACAGTTTAGACTA
CAATGAGAGGCTCTGGGCTTGGGAAAGCTGGAGATCTGAGGTCGGCAAGCAGCTGAGGC
CATTATATGAAGAGTATGTGGTCTTGAAAAATGAGATGGCAAGAGCAAATCATTATGAGG
ACTATGGGGATTATTGGAGAGGAGACTATGAAGTAAATGGGGTAGATGGCTATGACTACA
GCCGCGGCCAGTTGATTGAAGATGTGGAACATACCTTTGAAGAGATTAAACCATTATATG
AACATCTTCATGCCTATGTGAGGGCAAAGTTGATGAATGCCTATCCTTCCTATATCAGTCC
AATTGGATGCCTCCCTGCTCATTTGCTTGGTGATATGTGGGGTAGATTTTGGACAAATCTG
TACTCTTTGACAGTTCCCTTTGGACAGAAACCAAACATAGATGTTACTGATGCAATGGTGG
ACCAGGCCTGGGATGCACAGAGAATATTCAAGGAGGCCGAGAAGTTCTTTGTATCTGTTG
GTCTTCCTAATATGACTCAAGGATTCTGGGAAAATTCCATGCTAACGGACCCAGGAAATG
TTCAGAAAGCAGTCTGCCATCCCACAGCTTGGGACCTGGGGAAGGGCGACTTCAGGATCC
TTATGTGCACAAAGGTGACAATGGACGACTTCCTGACAGCTCATCATGAGATGGGGCATA
TCCAGTATGATATGGCATATGCTGCACAACCTTTTCTGCTAAGAAATGGAGCTAATGAAG
GATTCCATGAAGCTGTTGGGGAAATCATGTCACTTTCTGCAGCCACACCTAAGCATTTAAA
ATCCATTGGTCTTCTGTCACCCGATTTTCAAGAAGACAATGAAACAGAAATAAACTTCCTG
CTCAAACAAGCACTCACGATTGTTGGGACTCTGCCATTTACTTACATGTTAGAGAAGTGGA
GGTGGATGGTCTTTAAAGGGGAAATTCCCAAAGACCAGTGGATGAAAAAGTGGTGGGAG
ATGAAGCGAGAGATAGTTGGGGTGGTGGAACCTGTGCCCCATGATGAAACATACTGTGAC
CCCGCATCTCTGTTCCATGTTTCTAATGATTACTCATTCATTCGATATTACACAAGGACCCT
TTACCAATTCCAGTTTCAAGAAGCACTTTGTCAAGCAGCTAAACATGAAGGCCCTCTGCAC
AAATGTGACATCTCAAACTCTACAGAAGCTGGACAGAAACTGTTCAATATGCTGAGGCTT
GGAAAATCAGAACCCTGGACCCTAGCATTGGAAAATGTTGTAGGAGCAAAGAACATGAA
TGTAAGGCCACTGCTCAACTACTTTGAGCCCTTATTTACCTGGCTGAAAGACCAGAACAAG
AATTCTTTTGTGGGATGGAGTACCGACTGGAGTCCATATGCAGACGGATCTGCCCCTGCCC
CTGCTCCAGCTCCCGCCCCTGCCCCTGCCCCTGCCCCTGCCCCTGCCCCTGCTCCCGCCCCT
GCTCCAGCCCCTGCCCCCGGCAGCGGCTACATCCCCGAGGCCCCCAGGGACGGCCAGGCC
TACGTGAGGAAGGACGGCGAGTGGGTGCTGCTGAGCACCTTCCTG
ACE2-AP15-Foldon Protein Sequence
(SEQ ID NO: 29)
QSTIEEQAKTFLDKFNHEAEDLFYQSSLASWNYNTNITEENVQNMNNAGDKWSAFLKEQSTL
AQMYPLQEIQNLTVKLQLQALQQNGSSVLSEDKSKRLNTILNTMSTIYSTGKVCNPDNPQECL
LLEPGLNEIMANSLDYNERLWAWESWRSEVGKQLRPLYEEYVVLKNEMARANHYEDYGDY
WRGDYEVNGVDGYDYSRGQLIEDVEHTFEEIKPLYEHLHAYVRAKLMNAYPSYISPIGCLPAH
LLGDMWGRFWTNLYSLTVPFGQKPNIDVTDAMVDQAWDAQRIFKEAEKFFVSVGLPNMTQG
FWENSMLTDPGNVQKAVCHPTAWDLGKGDFRILMCTKVTMDDFLTAHHEMGHIQYDMAYA
AQPFLLRNGANEGFHEAVGEIMSLSAATPKHLKSIGLLSPDFQEDNETEINFLLKQALTIVGTLP
FTYMLEKWRWMVFKGEIPKDQWMKKWWEMKREIVGVVEPVPHDETYCDPASLFHVSNDYS
FIRYYTRTLYQFQFQEALCQAAKHEGPLHKCDISNSTEAGQKLFNMLRLGKSEPWTLALENVV
GAKNMNVRPLLNYFEPLFTWLKDQNKNSFVGWSTDWSPYADGSAPAPAPAPAPAPAPAPAP
APAPAPAPAPAPGSGYIPEAPRDGQAYVRKDGEWVLLSTFL
ACE2 M1-AP15-Foldon DNA Sequence
(SEQ ID NO: 30)
CAGTCCACCATTGAGGAACAGGCCAAGACATTTTTGGACttcTTTAACatccaaGCCGAAGACC
TGTTCTATCAAAGTTCACTTGCTTCTTGGAATTATAACACCAATATTACTGAAGAGAATGT
CCAAAACATGAATAATGCTGGGGACAAATGGTCTGCCTTTTTAAAGGAACAGTCCACACT
TGCCCAAATGTATCCACTACAAGAAATTCAGAATCTCACAGTCAAGCTTCAGCTGCAGGC
TCTTCAGCAAAATGGGTCTTCAGTGCTCTCAGAAGACAAGAGCAAACGGTTGAACACAAT
TCTAAATACAATGAGCACCATCTACAGTACTGGAAAAGTTTGTAACCCAGATAATCCACA
AGAATGCTTATTACTTGAACCAGGTTTGAATGAAATAATGGCAAACAGTTTAGACTACAA
TGAGAGGCTCTGGGCTTGGGAAAGCTGGAGATCTGAGGTCGGCAAGCAGCTGAGGCCATT
ATATGAAGAGTATGTGGTCTTGAAAAATGAGATGGCAAGAGCAAATCATTATGAGGACTA
TGGGGATTATTGGAGAGGAGACTATGAAGTAAATGGGGTAGATGGCTATGACTACAGCCG
CGGCCAGTTGATTGAAGATGTGGAACATACCTTTGAAGAGATTAAACCATTATATGAACA
TCTTCATGCCTATGTGAGGGCAAAGTTGATGAATGCCTATCCTTCCTATATCAGTCCAATT
GGATGCCTCCCTGCTCATTTGCTTGGTGATATGTGGGGTAGATTTTGGACAAATCTGTACT
CTTTGACAGTTCCCTTTGGACAGAAACCAAACATAGATGTTACTGATGCAATGGTGGACC
AGGCCTGGGATGCACAGAGAATATTCAAGGAGGCCGAGAAGTTCTTTGTATCTGTTGGTC
TTCCTAATATGACTCAAGGATTCTGGGAAAATTCCATGCTAACGGACCCAGGAAATGTTC
AGAAAGCAGTCTGCCATCCCACAGCTTGGGACCTGGGGAAGGGCGACTTCAGGATCCTTA
TGTGCACAAAGGTGACAATGGACGACTTCCTGACAGCTCATCATGAGATGGGGCATATCC
AGTATGATATGGCATATGCTGCACAACCTTTTCTGCTAAGAAATGGAGCTAATGAAGGAT
TCCATGAAGCTGTTGGGGAAATCATGTCACTTTCTGCAGCCACACCTAAGCATTTAAAATC
CATTGGTCTTCTGTCACCCGATTTTCAAGAAGACAATGAAACAGAAATAAACTTCCTGCTC
AAACAAGCACTCACGATTGTTGGGACTCTGCCATTTACTTACATGTTAGAGAAGTGGAGG
TGGATGGTCTTTAAAGGGGAAATTCCCAAAGACCAGTGGATGAAAAAGTGGTGGGAGAT
GAAGCGAGAGATAGTTGGGGTGGTGGAACCTGTGCCCCATGATGAAACATACTGTGACCC
CGCATCTCTGTTCCATGTTTCTAATGATTACTCATTCATTCGATATTACACAAGGACCCTTT
ACCAATTCCAGTTTCAAGAAGCACTTTGTCAAGCAGCTAAACATGAAGGCCCTCTGCACA
AATGTGACATCTCAAACTCTACAGAAGCTGGACAGAAACTGTTCAATATGCTGAGGCTTG
GAAAATCAGAACCCTGGACCCTAGCATTGGAAAATGTTGTAGGAGCAAAGAACATGAAT
GTAAGGCCACTGCTCAACTACTTTGAGCCCTTATTTACCTGGCTGAAAGACCAGAACAAG
AATTCTTTTGTGGGATGGAGTACCGACTGGAGTCCATATGCAGACGGATCTGCCCCTGCCC
CTGCTCCAGCTCCCGCCCCTGCCCCTGCCCCTGCCCCTGCCCCTGCCCCTGCTCCCGCCCCT
GCTCCAGCCCCTGCCCCCGGCAGCGGCTACATCCCCGAGGCCCCCAGGGACGGCCAGGCC
TACGTGAGGAAGGACGGCGAGTGGGTGCTGCTGAGCACCTTCCTGGGCGGCAGA
ACE2 M1-AP15-Foldon Protein Sequence
(SEQ ID NO: 31)
QSTIEEQAKTFLDFFNIQAEDLFYQSSLASWNYNTNITEENVQNMNNAGDKWSAFLKEQSTLA
QMYPLQEIQNLTVKLQLQALQQNGSSVLSEDKSKRLNTILNTMSTIYSTGKVCNPDNPQECLL
LEPGLNEIMANSLDYNERLWAWESWRSEVGKQLRPLYEEYVVLKNEMARANHYEDYGDYW
RGDYEVNGVDGYDYSRGQLIEDVEHTFEEIKPLYEHLHAYVRAKLMNAYPSYISPIGCLPAHL
LGDMWGRFWTNLYSLTVPFGQKPNIDVTDAMVDQAWDAQRIFKEAEKFFVSVGLPNMTQGF
WENSMLTDPGNVQKAVCHPTAWDLGKGDFRILMCTKVTMDDFLTAHHEMGHIQYDMAYA
AQPFLLRNGANEGFHEAVGEIMSLSAATPKHLKSIGLLSPDFQEDNETEINFLLKQALTIVGTLP
FTYMLEKWRWMVFKGEIPKDQWMKKWWEMKREIVGVVEPVPHDETYCDPASLFHVSNDYS
FIRYYTRTLYQFQFQEALCQAAKHEGPLHKCDISNSTEAGQKLFNMLRLGKSEPWTLALENVV
GAKNMNVRPLLNYFEPLFTWLKDQNKNSFVGWSTDWSPYADGSAPAPAPAPAPAPAPAPAP
APAPAPAPAPAPGSGYIPEAPRDGQAYVRKDGEWVLLSTFL
ACE2 M2-AP15-Foldon DNA Sequence
(SEQ ID NO: 32)
CAGTCCACCATTGAGGAACAGGCCAAGtacTTTTTGGACAAGTTTAACCACGAAGCCGAAG
ACCTGTTCTATCAAAGTTCACTTGCTTCTTGGAATTATAACACCAATATTACTGAAGAGAA
TGTCCAAAACATGAATAATGCTGGGGACAAATGGTCTGCCTTTTTAAAGGAACAGTCCAC
AtacGCCCAAATGTATCCACTACAAGAAATTCAGAATCTCACAGTCAAGCTTCAGCTGCAG
GCTCTTCAGCAAAATGGGTCTTCAGTGCTCTCAGAAGACAAGAGCAAACGGTTGAACACA
ATTCTAAATACAATGAGCACCATCTACAGTACTGGAAAAGTTTGTAACCCAGATAATCCA
CAAGAATGCTTATTACTTGAACCAGGTTTGAATGAAATAATGGCAAACAGTTTAGACTAC
AATGAGAGGCTCTGGGCTTGGGAAAGCTGGAGATCTGAGGTCGGCAAGCAGCTGAGGCC
ATTATATGAAGAGTATGTGGTCTTGAAAAATGAGATGGCAAGAGCAAATCATTATGAGGA
CTATGGGGATTATTGGAGAGGAGACTATGAAGTAAATGGGGTAGATGGCTATGACTACAG
CCGCGGCCAGTTGATTGAAGATGTGGAACATACCTTTGAAGAGATTAAACCATTATATGA
ACATCTTCATGCCTATGTGAGGGCAAAGTTGATGAATGCCTATCCTTCCTATATCAGTCCA
ATTGGATGCCTCCCTGCTCATTTGCTTGGTGATATGTGGGGTAGATTTTGGACAAATCTGT
ACTCTTTGACAGTTCCCTTTGGACAGAAACCAAACATAGATGTTACTGATGCAATGGTGGA
CCAGGCCTGGGATGCACAGAGAATATTCAAGGAGGCCGAGAAGTTCTTTGTATCTGTTGG
TCTTCCTAATATGACTCAAGGATTCTGGGAAtatTCCATGCTAACGGACCCAGGAAATGTTC
AGAAAGCAGTCTGCCATCCCACAGCTTGGGACCTGGGGAAGGGCGACTTCAGGATCCTTA
TGTGCACAAAGGTGACAATGGACGACTTCCTGACAGCTCATCATGAGATGGGGCATATCC
AGTATGATATGGCATATGCTGCACAACCTTTTCTGCTAAGAAATGGAGCTAATGAAGGAT
TCCATGAAGCTGTTGGGGAAATCATGTCACTTTCTGCAGCCACACCTAAGCATTTAAAATC
CATTGGTCTTCTGTCACCCGATTTTCAAGAAGACAATGAAACAGAAATAAACTTCCTGCTC
AAACAAGCACTCACGATTGTTGGGACTCTGCCATTTACTTACATGTTAGAGAAGTGGAGG
TGGATGGTCTTTAAAGGGGAAATTCCCAAAGACCAGTGGATGAAAAAGTGGTGGGAGAT
GAAGCGAGAGATAGTTGGGGTGGTGGAACCTGTGCCCCATGATGAAACATACTGTGACCC
CGCATCTCTGTTCCATGTTTCTAATGATTACTCATTCATTCGATATTACACAAGGACCCTTT
ACCAATTCCAGTTTCAAGAAGCACTTTGTCAAGCAGCTAAACATGAAGGCCCTCTGCACA
AATGTGACATCTCAAACTCTACAGAAGCTGGACAGAAACTGTTCAATATGCTGAGGCTTG
GAAAATCAGAACCCTGGACCCTAGCATTGGAAAATGTTGTAGGAGCAAAGAACATGAAT
GTAAGGCCACTGCTCAACTACTTTGAGCCCTTATTTACCTGGCTGAAAGACCAGAACAAG
AATTCTTTTGTGGGATGGAGTACCGACTGGAGTCCATATGCAGACGGATCTGCCCCTGCCC
CTGCTCCAGCTCCCGCCCCTGCCCCTGCCCCTGCCCCTGCCCCTGCCCCTGCTCCCGCCCCT
GCTCCAGCCCCTGCCCCCGGCAGCGGCTACATCCCCGAGGCCCCCAGGGACGGCCAGGCC
TACGTGAGGAAGGACGGCGAGTGGGTGCTGCTGAGCACCTTCCTGGGCGGCAGA
ACE2 M2-AP15-Foldon Protein Sequence
(SEQ ID NO: 33)
QSTIEEQAKYFLDKFNHEAEDLFYQSSLASWNYNTNITEENVQNMNNAGDKWSAFLKEQSTY
AQMYPLQEIQNLTVKLQLQALQQNGSSVLSEDKSKRLNTILNTMSTIYSTGKVCNPDNPQECL
LLEPGLNEIMANSLDYNERLWAWESWRSEVGKQLRPLYEEYVVLKNEMARANHYEDYGDY
WRGDYEVNGVDGYDYSRGQLIEDVEHTFEEIKPLYEHLHAYVRAKLMNAYPSYISPIGCLPAH
LLGDMWGRFWTNLYSLTVPFGQKPNIDVTDAMVDQAWDAQRIFKEAEKFFVSVGLPNMTQG
FWEYSMLTDPGNVQKAVCHPTAWDLGKGDFRILMCTKVTMDDFLTAHHEMGHIQYDMAYA
AQPFLLRNGANEGFHEAVGEIMSLSAATPKHLKSIGLLSPDFQEDNETEINFLLKQALTIVGTLP
FTYMLEKWRWMVFKGEIPKDQWMKKWWEMKREIVGVVEPVPHDETYCDPASLFHVSNDYS
FIRYYTRTLYQFQFQEALCQAAKHEGPLHKCDISNSTEAGQKLFNMLRLGKSEPWTLALENVV
GAKNMNVRPLLNYFEPLFTWLKDQNKNSFVGWSTDWSPYADGSAPAPAPAPAPAPAPAPAP
APAPAPAPAPAPGSGYIPEAPRDGQAYVRKDGEWVLLSTFL
ACE2 M3-AP15-Foldon DNA Sequence
(SEQ ID NO: 34)
CAGTCCACCATTGAGGAACAGGCCAAGtacTTTTTGGACAAGTTTAACgctGAAGCCGAAGA
CCTGTTCTATCAAAGTTCACTTGCTTCTTGGAATTATAACACCAATATTACTGAAGAGAAT
GTCCAAAACATGAATAATGCTGGGGACAAATGGTCTGCCTTTTTAAAGGAACAGTCCACA
CTTGCCCAAATGTATCCACTACAAGAAATTCAGAATCTCACAGTCAAGCTTCAGCTGCAG
GCTCTTCAGCAAAATGGGTCTTCAGTGCTCTCAGAAGACAAGAGCAAACGGTTGAACACA
ATTCTAAATACAATGAGCACCATCTACAGTACTGGAAAAGTTTGTAACCCAGATAATCCA
CAAGAATGCTTATTACTTGAACCAGGTTTGAATGAAATAATGGCAAACAGTTTAGACTAC
AATGAGAGGCTCTGGGCTTGGGAAAGCTGGAGATCTGAGGTCGGCAAGCAGCTGAGGCC
ATTATATGAAGAGTATGTGGTCTTGAAAAATGAGATGGCAAGAGCAAATCATTATGAGGA
CTATGGGGATTATTGGAGAGGAGACTATGAAGTAAATGGGGTAGATGGCTATGACTACAG
CCGCGGCCAGTTGATTGAAGATGTGGAACATACCTTTGAAGAGATTAAACCATTATATGA
ACATCTTCATGCCTATGTGAGGGCAAAGTTGATGAATGCCTATCCTTCCTATATCAGTCCA
ATTGGATGCCTCCCTGCTCATTTGCTTGGTGATATGTGGGGTAGATTTTGGACAAATCTGT
ACTCTTTGACAGTTCCCTTTGGACAGAAACCAAACATAGATGTTACTGATGCAATGGTGGA
CCAGGCCTGGGATGCACAGAGAATATTCAAGGAGGCCGAGAAGTTCTTTGTATCTGTTGG
TCTTCCTAATATGACTCAAGGATTCTGGGAAAATTCCATGCTAACGGACCCAGGAAATGTT
CAGAAAGCAGTCTGCCATCCCACAGCTTGGGACCTGGGGAAGGGCGACTTCAGGATCCTT
ATGTGCACAAAGGTGACAATGGACGACTTCCTGACAGCTCATCATGAGATGGGGCATATC
CAGTATGATATGGCATATGCTGCACAACCTTTTCTGCTAAGAAATGGAGCTAATGAAGGA
TTCCATGAAGCTGTTGGGGAAATCATGTCACTTTCTGCAGCCACACCTAAGCATTTAAAAT
CCATTGGTCTTCTGTCACCCGATTTTCAAGAAGACAATGAAACAGAAATAAACTTCCTGCT
CAAACAAGCACTCACGATTGTTGGGACTCTGCCATTTACTTACATGTTAGAGAAGTGGAG
GTGGATGGTCTTTAAAGGGGAAATTCCCAAAGACCAGTGGATGAAAAAGTGGTGGGAGA
TGAAGCGAGAGATAGTTGGGGTGGTGGAACCTGTGCCCCATGATGAAACATACTGTGACC
CCGCATCTCTGTTCCATGTTTCTAATGATTACTCATTCATTCGATATTACACAAGGACCCTT
TACCAATTCCAGTTTCAAGAAGCACTTTGTCAAGCAGCTAAACATGAAGGCCCTCTGCAC
AAATGTGACATCTCAAACTCTACAGAAGCTGGACAGAAACTGTTCAATATGCTGAGGCTT
GGAAAATCAGAACCCTGGACCCTAGCATTGGAAAATGTTGTAGGAGCAAAGAACATGAA
TGTAAGGCCACTGCTCAACTACTTTGAGCCCTTATTTACCTGGCTGAAAGACCAGAACAAG
AATTCTTTTGTGGGATGGAGTACCGACTGGAGTCCATATGCAGACGGATCTGCCCCTGCCC
CTGCTCCAGCTCCCGCCCCTGCCCCTGCCCCTGCCCCTGCCCCTGCCCCTGCTCCCGCCCCT
GCTCCAGCCCCTGCCCCCGGCAGCGGCTACATCCCCGAGGCCCCCAGGGACGGCCAGGCC
TACGTGAGGAAGGACGGCGAGTGGGTGCTGCTGAGCACCTTCCTGGGCGGCAGA
ACE2 M3-AP15-Foldon Protein Sequence
(SEQ ID NO: 35)
QSTIEEQAKYFLDKFNAEAEDLFYQSSLASWNYNTNITEENVQNMNNAGDKWSAFLKEQSTL
AQMYPLQEIQNLTVKLQLQALQQNGSSVLSEDKSKRLNTILNTMSTIYSTGKVCNPDNPQECL
LLEPGLNEIMANSLDYNERLWAWESWRSEVGKQLRPLYEEYVVLKNEMARANHYEDYGDY
WRGDYEVNGVDGYDYSRGQLIEDVEHTFEEIKPLYEHLHAYVRAKLMNAYPSYISPIGCLPAH
LLGDMWGRFWTNLYSLTVPFGQKPNIDVTDAMVDQAWDAQRIFKEAEKFFVSVGLPNMTQG
FWENSMLTDPGNVQKAVCHPTAWDLGKGDFRILMCTKVTMDDFLTAHHEMGHIQYDMAYA
AQPFLLRNGANEGFHEAVGEIMSLSAATPKHLKSIGLLSPDFQEDNETEINFLLKQALTIVGTLP
FTYMLEKWRWMVFKGEIPKDQWMKKWWEMKREIVGVVEPVPHDETYCDPASLFHVSNDYS
FIRYYTRTLYQFQFQEALCQAAKHEGPLHKCDISNSTEAGQKLFNMLRLGKSEPWTLALENVV
GAKNMNVRPLLNYFEPLFTWLKDQNKNSFVGWSTDWSPYADGSAPAPAPAPAPAPAPAPAP
APAPAPAPAPAPGSGYIPEAPRDGQAYVRKDGEWVLLSTFL
ACE2 M4-AP15-Foldon DNA Sequence
(SEQ ID NO: 36)
CAGTCCACCATTGAGGAACAGGCCAAGtacTTTTTGGACttcTTTAACatccaaGCCGAAGACCT
GTTCTATCAAAGTTCACTTGCTTCTTGGAATTATAACACCAATATTACTGAAGAGAATGTC
CAAAACATGAATAATGCTGGGGACAAATGGTCTGCCTTTTTAAAGGAACAGTCCACAtacG
CCCAAATGTATCCACTACAAGAAATTCAGAATCTCACAGTCAAGCTTCAGCTGCAGGCTCT
TCAGCAAAATGGGTCTTCAGTGCTCTCAGAAGACAAGAGCAAACGGTTGAACACAATTCT
AAATACAATGAGCACCATCTACAGTACTGGAAAAGTTTGTAACCCAGATAATCCACAAGA
ATGCTTATTACTTGAACCAGGTTTGAATGAAATAATGGCAAACAGTTTAGACTACAATGA
GAGGCTCTGGGCTTGGGAAAGCTGGAGATCTGAGGTCGGCAAGCAGCTGAGGCCATTATA
TGAAGAGTATGTGGTCTTGAAAAATGAGATGGCAAGAGCAAATCATTATGAGGACTATGG
GGATTATTGGAGAGGAGACTATGAAGTAAATGGGGTAGATGGCTATGACTACAGCCGCGG
CCAGTTGATTGAAGATGTGGAACATACCTTTGAAGAGATTAAACCATTATATGAACATCTT
CATGCCTATGTGAGGGCAAAGTTGATGAATGCCTATCCTTCCTATATCAGTCCAATTGGAT
GCCTCCCTGCTCATTTGCTTGGTGATATGTGGGGTAGATTTTGGACAAATCTGTACTCTTTG
ACAGTTCCCTTTGGACAGAAACCAAACATAGATGTTACTGATGCAATGGTGGACCAGGCC
TGGGATGCACAGAGAATATTCAAGGAGGCCGAGAAGTTCTTTGTATCTGTTGGTCTTCCTA
ATATGACTCAAGGATTCTGGGAAtatTCCATGCTAACGGACCCAGGAAATGTTCAGAAAGC
AGTCTGCCATCCCACAGCTTGGGACCTGGGGAAGGGCGACTTCAGGATCCTTATGTGCAC
AAAGGTGACAATGGACGACTTCCTGACAGCTCATCATGAGATGGGGCATATCCAGTATGA
TATGGCATATGCTGCACAACCTTTTCTGCTAAGAAATGGAGCTAATGAAGGATTCCATGA
AGCTGTTGGGGAAATCATGTCACTTTCTGCAGCCACACCTAAGCATTTAAAATCCATTGGT
CTTCTGTCACCCGATTTTCAAGAAGACAATGAAACAGAAATAAACTTCCTGCTCAAACAA
GCACTCACGATTGTTGGGACTCTGCCATTTACTTACATGTTAGAGAAGTGGAGGTGGATGG
TCTTTAAAGGGGAAATTCCCAAAGACCAGTGGATGAAAAAGTGGTGGGAGATGAAGCGA
GAGATAGTTGGGGTGGTGGAACCTGTGCCCCATGATGAAACATACTGTGACCCCGCATCT
CTGTTCCATGTTTCTAATGATTACTCATTCATTCGATATTACACAAGGACCCTTTACCAATT
CCAGTTTCAAGAAGCACTTTGTCAAGCAGCTAAACATGAAGGCCCTCTGCACAAATGTGA
CATCTCAAACTCTACAGAAGCTGGACAGAAACTGTTCAATATGCTGAGGCTTGGAAAATC
AGAACCCTGGACCCTAGCATTGGAAAATGTTGTAGGAGCAAAGAACATGAATGTAAGGCC
ACTGCTCAACTACTTTGAGCCCTTATTTACCTGGCTGAAAGACCAGAACAAGAATTCTTTT
GTGGGATGGAGTACCGACTGGAGTCCATATGCAGACGGATCTGCCCCTGCCCCTGCTCCA
GCTCCCGCCCCTGCCCCTGCCCCTGCCCCTGCCCCTGCCCCTGCTCCCGCCCCTGCTCCAGC
CCCTGCCCCCGGCAGCGGCTACATCCCCGAGGCCCCCAGGGACGGCCAGGCCTACGTGAG
GAAGGACGGCGAGTGGGTGCTGCTGAGCACCTTCCTGGGCGGCAGA
ACE2 M4-AP15-Foldon Protein Sequence
(SEQ ID NO: 37)
QSTIEEQAKYFLDFFNIQAEDLFYQSSLASWNYNTNITEENVQNMNNAGDKWSAFLKEQSTY
AQMYPLQEIQNLTVKLQLQALQQNGSSVLSEDKSKRLNTILNTMSTIYSTGKVCNPDNPQECL
LLEPGLNEIMANSLDYNERLWAWESWRSEVGKQLRPLYEEYVVLKNEMARANHYEDYGDY
WRGDYEVNGVDGYDYSRGQLIEDVEHTFEEIKPLYEHLHAYVRAKLMNAYPSYISPIGCLPAH
LLGDMWGRFWTNLYSLTVPFGQKPNIDVTDAMVDQAWDAQRIFKEAEKFFVSVGLPNMTQG
FWEYSMLTDPGNVQKAVCHPTAWDLGKGDFRILMCTKVTMDDFLTAHHEMGHIQYDMAYA
AQPFLLRNGANEGFHEAVGEIMSLSAATPKHLKSIGLLSPDFQEDNETEINFLLKQALTIVGTLP
FTYMLEKWRWMVFKGEIPKDQWMKKWWEMKREIVGVVEPVPHDETYCDPASLFHVSNDYS
FIRYYTRTLYQFQFQEALCQAAKHEGPLHKCDISNSTEAGQKLFNMLRLGKSEPWTLALENVV
GAKNMNVRPLLNYFEPLFTWLKDQNKNSFVGWSTDWSPYADGSAPAPAPAPAPAPAPAPAP
APAPAPAPAPAPGSGYIPEAPRDGQAYVRKDGEWVLLSTFL
ACE2 M5-AP15-Foldon DNA Sequence
(SEQ ID NO: 38)
CAGTCCACCATTGAGGAACAGGCCAAGtacTTTTTGGACAAGTTTAACgctGAAGCCGAAGA
CCTGTTCTATCAAAGTTCACTTGCTTCTTGGAATTATAACACCAATATTACTGAAGAGAAT
GTCCAAAACATGAATAATGCTGGGGACAAATGGTCTGCCTTTTTAAAGGAACAGTCCACAt
acGCCCAAATGTATCCACTACAAGAAATTCAGAATCTCACAGTCAAGCTTCAGCTGCAGGC
TCTTCAGCAAAATGGGTCTTCAGTGCTCTCAGAAGACAAGAGCAAACGGTTGAACACAAT
TCTAAATACAATGAGCACCATCTACAGTACTGGAAAAGTTTGTAACCCAGATAATCCACA
AGAATGCTTATTACTTGAACCAGGTTTGAATGAAATAATGGCAAACAGTTTAGACTACAA
TGAGAGGCTCTGGGCTTGGGAAAGCTGGAGATCTGAGGTCGGCAAGCAGCTGAGGCCATT
ATATGAAGAGTATGTGGTCTTGAAAAATGAGATGGCAAGAGCAAATCATTATGAGGACTA
TGGGGATTATTGGAGAGGAGACTATGAAGTAAATGGGGTAGATGGCTATGACTACAGCCG
CGGCCAGTTGATTGAAGATGTGGAACATACCTTTGAAGAGATTAAACCATTATATGAACA
TCTTCATGCCTATGTGAGGGCAAAGTTGATGAATGCCTATCCTTCCTATATCAGTCCAATT
GGATGCCTCCCTGCTCATTTGCTTGGTGATATGTGGGGTAGATTTTGGACAAATCTGTACT
CTTTGACAGTTCCCTTTGGACAGAAACCAAACATAGATGTTACTGATGCAATGGTGGACC
AGGCCTGGGATGCACAGAGAATATTCAAGGAGGCCGAGAAGTTCTTTGTATCTGTTGGTC
TTCCTAATATGACTCAAGGATTCTGGGAAtatTCCATGCTAACGGACCCAGGAAATGTTCAG
AAAGCAGTCTGCCATCCCACAGCTTGGGACCTGGGGAAGGGCGACTTCAGGATCCTTATG
TGCACAAAGGTGACAATGGACGACTTCCTGACAGCTCATCATGAGATGGGGCATATCCAG
TATGATATGGCATATGCTGCACAACCTTTTCTGCTAAGAAATGGAGCTAATGAAGGATTCC
ATGAAGCTGTTGGGGAAATCATGTCACTTTCTGCAGCCACACCTAAGCATTTAAAATCCAT
TGGTCTTCTGTCACCCGATTTTCAAGAAGACAATGAAACAGAAATAAACTTCCTGCTCAAA
CAAGCACTCACGATTGTTGGGACTCTGCCATTTACTTACATGTTAGAGAAGTGGAGGTGG
ATGGTCTTTAAAGGGGAAATTCCCAAAGACCAGTGGATGAAAAAGTGGTGGGAGATGAA
GCGAGAGATAGTTGGGGTGGTGGAACCTGTGCCCCATGATGAAACATACTGTGACCCCGC
ATCTCTGTTCCATGTTTCTAATGATTACTCATTCATTCGATATTACACAAGGACCCTTTACC
AATTCCAGTTTCAAGAAGCACTTTGTCAAGCAGCTAAACATGAAGGCCCTCTGCACAAAT
GTGACATCTCAAACTCTACAGAAGCTGGACAGAAACTGTTCAATATGCTGAGGCTTGGAA
AATCAGAACCCTGGACCCTAGCATTGGAAAATGTTGTAGGAGCAAAGAACATGAATGTAA
GGCCACTGCTCAACTACTTTGAGCCCTTATTTACCTGGCTGAAAGACCAGAACAAGAATTC
TTTTGTGGGATGGAGTACCGACTGGAGTCCATATGCAGACGGATCTGCCCCTGCCCCTGCT
CCAGCTCCCGCCCCTGCCCCTGCCCCTGCCCCTGCCCCTGCCCCTGCTCCCGCCCCTGCTCC
AGCCCCTGCCCCCGGCAGCGGCTACATCCCCGAGGCCCCCAGGGACGGCCAGGCCTACGT
GAGGAAGGACGGCGAGTGGGTGCTGCTGAGCACCTTCCTGGGCGGCAGA
ACE2 M5-AP15-Foldon Protein Sequence
(SEQ ID NO: 39)
QSTIEEQAKYFLDKFNAEAEDLFYQSSLASWNYNTNITEENVQNMNNAGDKWSAFLKEQSTY
AQMYPLQEIQNLTVKLQLQALQQNGSSVLSEDKSKRLNTILNTMSTIYSTGKVCNPDNPQECL
LLEPGLNEIMANSLDYNERLWAWESWRSEVGKQLRPLYEEYVVLKNEMARANHYEDYGDY
WRGDYEVNGVDGYDYSRGQLIEDVEHTFEEIKPLYEHLHAYVRAKLMNAYPSYISPIGCLPAH
LLGDMWGRFWTNLYSLTVPFGQKPNIDVTDAMVDQAWDAQRIFKEAEKFFVSVGLPNMTQG
FWEYSMLTDPGNVQKAVCHPTAWDLGKGDFRILMCTKVTMDDFLTAHHEMGHIQYDMAYA
AQPFLLRNGANEGFHEAVGEIMSLSAATPKHLKSIGLLSPDFQEDNETEINFLLKQALTIVGTLP
FTYMLEKWRWMVFKGEIPKDQWMKKWWEMKREIVGVVEPVPHDETYCDPASLFHVSNDYS
FIRYYTRTLYQFQFQEALCQAAKHEGPLHKCDISNSTEAGQKLFNMLRLGKSEPWTLALENVV
GAKNMNVRPLLNYFEPLFTWLKDQNKNSFVGWSTDWSPYADGSAPAPAPAPAPAPAPAPAP
APAPAPAPAPAPGSGYIPEAPRDGQAYVRKDGEWVLLSTFL
GS(EAAAK)₅GS linker aa sequence
(SEQ ID NO: 40)
GSEAAAKEAAAKEAAAKEAAAKEAAAKGS
AH5 linker aa sequence
(SEQ ID NO: 41)
GSAEAAAKEAAAKEAAAKEAAAKEAAAKAGS
GGGH5 linker aa sequence
(SEQ ID NO: 42)
GGGGSEAAAKEAAAKEAAAKEAAAKEAAAKGGGGS
H3 linker aa sequence
(SEQ ID NO: 43)
GSEAAAKEAAAKEAAAKGS
H4 linker aa sequence
(SEQ ID NO: 44)
GSEAAAKEAAAKEAAAKEAAAKGS
H6 linker aa sequence
(SEQ ID NO: 45)
GSEAAAKEAAAKEAAAKEAAAKEAAAKEAAAKGS
H7 linker aa sequence
(SEQ ID NO: 46)
GSEAAAKEAAAKEAAAKEAAAKEAAAKEAAAKEAAAKGS
AP12 linker aa sequence
(SEQ ID NO: 47)
GSAPAPAPAPAPAPAPAPAPAPAPAPGS
AP15 linker aa sequence
(SEQ ID NO: 48)
GSAPAPAPAPAPAPAPAPAPAPAPAPAPAPAPGS
(GGGGS)₅ linker aa sequence
(SEQ ID NO: 49)
GGGGSGGGGSGGGGSGGGGSGGGGS
(EAAAK)₅ linker aa sequence
(SEQ ID NO: 50)
EAAAKEAAAKEAAAKEAAAKEAAAK
ACE2 M1 Protein Sequence
(SEQ ID NO: 51)
QSTIEEQAKTFLDFFNIQAEDLFYQSSLASWNYNTNITEENVQNMNNAGDKWSAFLKEQSTLA
QMYPLQEIQNLTVKLQLQALQQNGSSVLSEDKSKRLNTILNTMSTIYSTGKVCNPDNPQECLL
LEPGLNEIMANSLDYNERLWAWESWRSEVGKQLRPLYEEYVVLKNEMARANHYEDYGDYW
RGDYEVNGVDGYDYSRGQLIEDVEHTFEEIKPLYEHLHAYVRAKLMNAYPSYISPIGCLPAHL
LGDMWGRFWTNLYSLTVPFGQKPNIDVTDAMVDQAWDAQRIFKEAEKFFVSVGLPNMTQGF
WENSMLTDPGNVQKAVCHPTAWDLGKGDFRILMCTKVTMDDFLTAHHEMGHIQYDMAYA
AQPFLLRNGANEGFHEAVGEIMSLSAATPKHLKSIGLLSPDFQEDNETEINFLLKQALTIVGTLP
FTYMLEKWRWMVFKGEIPKDQWMKKWWEMKREIVGVVEPVPHDETYCDPASLFHVSNDYS
FIRYYTRTLYQFQFQEALCQAAKHEGPLHKCDISNSTEAGQKLFNMLRLGKSEPWTLALENVV
GAKNMNVRPLLNYFEPLFTWLKDQNKNSFVGWSTDWSPYAD
ACE2 M2 Protein Sequence
(SEQ ID NO: 52)
QSTIEEQAKYFLDKFNHEAEDLFYQSSLASWNYNTNITEENVQNMNNAGDKWSAFLKEQSTY
AQMYPLQEIQNLTVKLQLQALQQNGSSVLSEDKSKRLNTILNTMSTIYSTGKVCNPDNPQECL
LLEPGLNEIMANSLDYNERLWAWESWRSEVGKQLRPLYEEYVVLKNEMARANHYEDYGDY
WRGDYEVNGVDGYDYSRGQLIEDVEHTFEEIKPLYEHLHAYVRAKLMNAYPSYISPIGCLPAH
LLGDMWGRFWTNLYSLTVPFGQKPNIDVTDAMVDQAWDAQRIFKEAEKFFVSVGLPNMTQG
FWEYSMLTDPGNVQKAVCHPTAWDLGKGDFRILMCTKVTMDDFLTAHHEMGHIQYDMAYA
AQPFLLRNGANEGFHEAVGEIMSLSAATPKHLKSIGLLSPDFQEDNETEINFLLKQALTIVGTLP
FTYMLEKWRWMVFKGEIPKDQWMKKWWEMKREIVGVVEPVPHDETYCDPASLFHVSNDYS
FIRYYTRTLYQFQFQEALCQAAKHEGPLHKCDISNSTEAGQKLFNMLRLGKSEPWTLALENVV
GAKNMNVRPLLNYFEPLFTWLKDQNKNSFVGWSTDWSPYAD
ACE2 M3 Protein Sequence
(SEQ ID NO: 53)
QSTIEEQAKYFLDKFNAEAEDLFYQSSLASWNYNTNITEENVQNMNNAGDKWSAFLKEQSTL
AQMYPLQEIQNLTVKLQLQALQQNGSSVLSEDKSKRLNTILNTMSTIYSTGKVCNPDNPQECL
LLEPGLNEIMANSLDYNERLWAWESWRSEVGKQLRPLYEEYVVLKNEMARANHYEDYGDY
WRGDYEVNGVDGYDYSRGQLIEDVEHTFEEIKPLYEHLHAYVRAKLMNAYPSYISPIGCLPAH
LLGDMWGRFWTNLYSLTVPFGQKPNIDVTDAMVDQAWDAQRIFKEAEKFFVSVGLPNMTQG
FWENSMLTDPGNVQKAVCHPTAWDLGKGDFRILMCTKVTMDDFLTAHHEMGHIQYDMAYA
AQPFLLRNGANEGFHEAVGEIMSLSAATPKHLKSIGLLSPDFQEDNETEINFLLKQALTIVGTLP
FTYMLEKWRWMVFKGEIPKDQWMKKWWEMKREIVGVVEPVPHDETYCDPASLFHVSNDYS
FIRYYTRTLYQFQFQEALCQAAKHEGPLHKCDISNSTEAGQKLFNMLRLGKSEPWTLALENVV
GAKNMNVRPLLNYFEPLFTWLKDQNKNSFVGWSTDWSPYAD
ACE2 M4 Protein Sequence
(SEQ ID NO: 54)
QSTIEEQAKYFLDFFNIQAEDLFYQSSLASWNYNTNITEENVQNMNNAGDKWSAFLKEQSTY
AQMYPLQEIQNLTVKLQLQALQQNGSSVLSEDKSKRLNTILNTMSTIYSTGKVCNPDNPQECL
LLEPGLNEIMANSLDYNERLWAWESWRSEVGKQLRPLYEEYVVLKNEMARANHYEDYGDY
WRGDYEVNGVDGYDYSRGQLIEDVEHTFEEIKPLYEHLHAYVRAKLMNAYPSYISPIGCLPAH
LLGDMWGRFWTNLYSLTVPFGQKPNIDVTDAMVDQAWDAQRIFKEAEKFFVSVGLPNMTQG
FWEYSMLTDPGNVQKAVCHPTAWDLGKGDFRILMCTKVTMDDFLTAHHEMGHIQYDMAYA
AQPFLLRNGANEGFHEAVGEIMSLSAATPKHLKSIGLLSPDFQEDNETEINFLLKQALTIVGTLP
FTYMLEKWRWMVFKGEIPKDQWMKKWWEMKREIVGVVEPVPHDETYCDPASLFHVSNDYS
FIRYYTRTLYQFQFQEALCQAAKHEGPLHKCDISNSTEAGQKLFNMLRLGKSEPWTLALENVV
GAKNMNVRPLLNYFEPLFTWLKDQNKNSFVGWSTDWSPYAD
ACE2 M5 Protein Sequence
(SEQ ID NO: 55)
QSTIEEQAKYFLDKFNAEAEDLFYQSSLASWNYNTNITEENVQNMNNAGDKWSAFLKEQSTY
AQMYPLQEIQNLTVKLQLQALQQNGSSVLSEDKSKRLNTILNTMSTIYSTGKVCNPDNPQECL
LLEPGLNEIMANSLDYNERLWAWESWRSEVGKQLRPLYEEYVVLKNEMARANHYEDYGDY
WRGDYEVNGVDGYDYSRGQLIEDVEHTFEEIKPLYEHLHAYVRAKLMNAYPSYISPIGCLPAH
LLGDMWGRFWTNLYSLTVPFGQKPNIDVTDAMVDQAWDAQRIFKEAEKFFVSVGLPNMTQG
FWEYSMLTDPGNVQKAVCHPTAWDLGKGDFRILMCTKVTMDDFLTAHHEMGHIQYDMAYA
AQPFLLRNGANEGFHEAVGEIMSLSAATPKHLKSIGLLSPDFQEDNETEINFLLKQALTIVGTLP
FTYMLEKWRWMVFKGEIPKDQWMKKWWEMKREIVGVVEPVPHDETYCDPASLFHVSNDYS
FIRYYTRTLYQFQFQEALCQAAKHEGPLHKCDISNSTEAGQKLFNMLRLGKSEPWTLALENVV
GAKNMNVRPLLNYFEPLFTWLKDQNKNSFVGWSTDWSPYAD

### REFERENCES

1. X.-L. Y. Peng Zhou, Xian-Guang Wang, Ben Hu, Lei Zhang, Wei Zhang, Hao-Rui Si, Yan Zhu, Bei Li, Chao-Lin Huang, Hui-Dong Chen, Jing Chen, Yun Luo, Hua Guo, Ren-Di Jiang, Mei-Qin Liu, Ying Chen, Xu-Rui Shen, Xi Wang, Xiao-Shuang Zheng, Kai Zhao, F. D. Quan-Jiao Chen, Lin-Lin Liu, Bing Yan, Fa-Xian Zhan, Yan-Yi Wang, Geng-Fu Xiao Zheng-Li Shi, A pneumonia outbreak associated with a new coronavirus of probable bat origin. Nature, (2020).
2. F. Li, W. Li, M. Farzan, S C Harrison, Structure of SARS coronavirus spike receptor-binding domain complexed with receptor. Science 309, 1864-1868 (2005).
3. D. R. Beniac, A. Andonov, E. Grudeski, T. F. Booth, Architecture of the SARS coronavirus prefusion spike. Nat Struct Mol Biol 13, 751-752 (2006).
4. N. W. Daniel Wrapp, Kizzmekia S. Corbett, Joiy A. Goldsmith, Ching-Lin Hsieh, Olubukola Abiona, Barney S. Graham, Jason S. McLellan, Cryo-EM structure of the 2019-nCoV spike in the prefusion conformation. Science, (2020).
5. W. H. Li et al., Angiotensin-converting enzyme 2 is a functional receptor for the SARS coronavirus. Nature 426, 450-454 (2003).
6. B. Ju et al., Human neutralizing antibodies elicited by SARS-CoV-2 infection. Nature 584, 115-119 (2020).
7. Y. Cao et al., Potent Neutralizing Antibodies against SARS-CoV-2 Identified by High-Throughput Single-Cell Sequencing of Convalescent Patients' B Cells. Cell 182, 73-84 e16 (2020).
8. Y. Wu et al., A noncompeting pair of human neutralizing antibodies block COVID-19 virus binding to its receptor ACE2. Science 368, 1274-1278 (2020).
9. A. Baum et al., Antibody cocktail to SARS-CoV-2 spike protein prevents rapid mutational escape seen with individual antibodies. Science, (2020).
10. J. Hansen et al., Studies in humanized mice and convalescent humans yield a SARS-CoV-2 antibody cocktail. Science, (2020).
11. J. W. Drake, J. J. Holland, Mutation rates among RNA viruses. P Natl Acad Sci USA 96, 13910-13913 (1999).
12. R. Sanjuan, M. R. Nebot, N. Chirico, L. M. Mansky, R. Belshaw, Viral mutation rates. J Viral 84, 9733-9748 (2010).
13. B. Korber et at , Tracking Changes in SARS-CoV-2 Spike: Evidence that D614G Increases Infectivity of the COVID-19 Virus. Cell, (2020).
14. J. W. Qianqian Li, Jianhui Nie, Li Zhang, Huan Hao, Shuo Liu, Chenyan Zhao, Qi Zhang, Huan Liu, Lingling Nie, Haiyang Qin, Meng Wang, Qiong Lu, Xiaoyu Li, Qiyu Sun, Junkai Liu, Linqi Zhang, Xuguang Li, Weijin Huang, Youchun Wang, The impact of mutations in SARS-CoV-2 spike on viral infectivity and antigenicity. Cell, (2020).
15. F. S. Yiska Weisblum, Fengwen Zhang, Justin DaSilva, Daniel Poston, Julio C. C. Lorenzi, Frauke Muecksch, Magdalena Rutkowska, Hans-Heinrich Hoffmann, Eleftherios Michailidis, Christian Gaebler, Marianna Agudelo, Alice Cho, Zijun Wang, Anna Gazumyan, Melissa Cipolla, Larry Luchsinger, Christopher D. Hillyer, Marina Caskey, Davide F. Robbiani, Charles M. Rice, Michel C. Nussenzweig, Theodora Hatziioannou, Paul D. Bieniasz, Escape from neutralizing antibodies by SARS-CoV-2 spike protein variants. bioRxiv preprint doi: https://doi.org/10.1101/2020.07.21.214759, (2020).
16. Z. Z. Junxian Ou, Ruixue Dai, Jing Zhang, Wendong Lan, Shan Zhao, Jianguo Wu, Donald Seto, Lilian Cui, Gong Zhang, Qiwei Zhang, Emergence of RBD mutations in circulating SARS-CoV-2 strains enhancing the structural stability and human ACE2 receptor affinity of the spike protein. bioRxiv doi: https://doi.org/10.1101/2020.03.15.991844, (2020).
17. K. Kuba et al., A crucial role of angiotensin converting enzyme 2 (ACE2) in SARS coronavirus-induced lung injury. Nat Med 11, 875-879 (2005).
18. I. Glowacka et al., Differential Downregulation of ACE2 by the Spike Proteins of Severe Acute Respiratory Syndrome Coronavirus and Human Coronavirus NL63. J Virol 84, 1198-1205 (2010).
19. Y. Liu et al., Clinical and biochemical indexes from 2019-nCoV infected patients linked to viral loads and lung injury. Sci China Life Sci 63, 364-374 (2020).
20. F. Huang et al., Angiotensin II plasma levels are linked to disease severity and predict fatal outcomes in H7N9-infected patients. Nat Commun 5, 3595 (2014).
21. Y. Imai et al., Angiotensin-converting enzyme 2 protects from severe acute lung failure. Nature 436, 112-116 (2005).
22. Z. Zou et al., Angiotensin-converting enzyme 2 protects from lethal avian influenza A H5N1 infections. Nat Commun 5, 3594 (2014).
23. A. Khan et al., A pilot clinical trial of recombinant human angiotensin-converting enzyme 2 in acute respiratory distress syndrome. Crit Care 21, (2017).
24. H. Hofmann et al., Susceptibility to SARS coronavirus S protein-driven infection correlates with expression of angiotensin converting enzyme 2 and infection can be blocked by soluble receptor. Biochem Bioph Res Co 319, 1216-1221 (2004).
25. C. Lei et al., Neutralization of SARS-CoV-2 spike pseudotyped virus by recombinant ACE2-Ig. Nat Commun 11, 2070 (2020).
26. V. Monteil et al., Inhibition of SARS-CoV-2 Infections in Engineered Human Tissues Using Clinical-Grade Soluble Human ACE2. Cell 181, 905-913 e907 (2020).
27. D. Wrapp et al., Cryo-EM structure of the 2019-nCoV spike in the prefusion conformation. Science 367, 1260-+(2020).
28. X. X. Z. Irene Lui, Shion A. Lim, Susanna K. Elledge, Paige Solomon, Nicholas J. Rettko, Beth Shoshana Zha, Lisa L. Kirkemo, Josef A. Gramespacher, Jia Liu, Frauke Muecksch, Julio Cesar Cetrulo Lorenzi, Fabian Schmidt, Yiska Weisblum, Davide F. Robbiani, Michel C. Nussenzweig, Theodora Hatziioannou, Paul D. Bieniasz, Oren S. Rosenburg, Kevin K. Leung, James A. Wells, Trimeric SARS-CoV-2 Spike interacts with dimeric ACE2 with limited intra-Spike avidity. bioRxiv doi: https://doi.org/10.1101/2020.05.21.109157., (2020).
29. K. K. Chan et al., Engineering human ACE2 to optimize binding to the spike protein of SARS coronavirus 2. Science, (2020).
30. H. W. Yujun Li, Xiaojuan Tang, Danting Ma, Chengzhi Du, Yifei Wang, Hong Pan, Qing Zou, Jie Zheng, Liangde Xu, Michael Farzan, Guocai Zhong, Potential host range of multiple SARS-like coronaviruses and an improved ACE2-Fc variant that is potent against both SARS-CoV-2 and SARS-CoV-1. BioRxiv doi: https://doi.org/10.1101/2020.04.10.032342, (2020).
31. X. Yang et al., Highly stable trimers formed by human immunodeficiency virus type 1 envelope glycoproteins fused with the trimeric motif of T4 bacteriophage fibritin. J Virol 76, 4634-4642 (2002).
32. S. Meier, S. Guthe, T. Kiefhaber, S. Grzesiek, Foldon, the natural trimerization domain of T4 fibritin, dissociates into a monomeric A-state form containing a stable beta-hairpin: atomic details of trimer dissociation and local beta-hairpin stability from residual dipolar couplings. J Mal Biol 344, 1051-1069 (2004).
33. J. A. Boice, G. R. Dieckmann, W. F. DeGrado, R. Fairman, Thermodynamic analysis of a designed three-stranded coiled coil. Biochemistry 35, 14480-14485 (1996).
34. J. M. Fletcher et al., A basis set of de novo coiled-coil peptide oligomers for rational protein design and synthetic biology. ACS Synth Biol 1, 240-250 (2012).
35. R. N. Kirchdoerfer et al., Stabilized coronavirus spikes are resistant to conformational changes induced by receptor recognition or proteolysis. Sci Rep 8, 15701 (2018).
36. W. Song, M. Gui, X. Wang, Y Xiang, Cryo-EM structure of the SARS coronavirus spike glycoprotein in complex with its host cell receptor ACE2. PLoS Pathog 14, e1007236 (2018).
37. Y. Cai et al., Distinct conformational states of SARS-CoV-2 spike protein. Science, (2020).
38. A. C. Walls et al., Structure, Function, and Antigenicity of the SARS-CoV-2 Spike Glycoprotein. Cell 181, 281-+(2020).
39. M. Gui et al., Cryo-electron microscopy structures of the SARS-CoV spike glycoprotein reveal a prerequisite conformational state for receptor binding. Cell Res 27, 119-129 (2017).
40. Y. Lin et al., Probing the structure of the SARS coronavirus using scanning electron microscopy. Antivir Ther 9, 287-289 (2004).
41. B. W. Neuman et al., Supramolecular architecture of severe acute respiratory syndrome coronavirus revealed by electron cryomicroscopy. J Virol 80, 7918-7928 (2006).
42. X. Chen, J. L. Zaro, W. C. Shen, Fusion protein linkers: property, design and functionality. Adv Drug Deliv Rev 65, 1357-1369 (2013).
43. W. M. F. B. Korber, S. Gnanakaran, H Yoon, J. Theiler, W. Abfalterer, N. Hengartner, E.E. Giorgi, T. Bhattacharya, B. Foley, K.M. Hastie, M.D. Parker, D.G. Partridge, C.M. Evans, T.M. Freeman, T.I. de Silva, C. McDanal, L.G. Perez, H. Tang, A. Moon-Walker, S.P. Whelan, C.C. LaBranche, E.O. Saphire, D.C. Montefiori, Tracking changes in SARS-CoV-2 Spike: evidence that D614G increases infectivity of the COVID-19 virus. Cell, (2020).
44. R. Yan et al., Structural basis for the recognition of SARS-CoV-2 by full-length human ACE2. Science 367, 1444-1448 (2020).
45. Q. H. Wang et al., Structural and Functional Basis of SARS-CoV-2 Entry by Using Human ACE2. Cell 181, 894-+(2020).
46. T. Zhou, Tsybovsky, Y., Olia, A. S., Gorman, J., Rapp, M. A., Cerutti, G., Katsamba, P. S., Nazzari, A., Schon, A., Wang, P.D., Bimela, J., Shi, W., Teng, I.T., Zhang, B., Boyington, J. C., Chuang, G.Y., Sampson, J. M., Sastry, M., Stephens, T., Stuckey, J., Wang, S., Friesner, R. A., Ho, D. D., Mascola, J. R., Shapiro, L., Kwong, P. D., A pH-dependent switch mediates conformational masking of SARS-CoV-2 spike. bioRxiv doi: https://doi.org/10.1101/2020.07.04.187989., (2020).
47. Y. W. C. Xu, C. Liu, C. Zhang, W. Han, X. Hong, Y. Wang, Q. Hong, S. Wang, Q. Zhao, Y. Wang, Y. Yang, K. Chen, W. Zheng, L. Kong, F. Wang, Q. Zuo, Z. Huang, Y. Cong, Conformational dynamics of SARS-CoV-2 trimeric spike glycoprotein in complex with receptor ACE2 revealed by cryo-EM. bioRxiv doi: https://doi.org/10.1101/2020.06.30.177097., (2020).
48. W. R. Strohl, Fusion Proteins for Half-Life Extension of Biologics as a Strategy to Make Biobetters. Biodrugs 29, 215-239 (2015).
49. e. a. A. Glasgow, Engineered ACE2 receptor traps potently neutralize SARS-CoV-2. bioRxiv doi: https://doi.org/10.1101/2020.07.31.231746, (2020).
50. Y. Bai, W. C. Shen, Improving the oral efficacy of recombinant granulocyte colony-stimulating factor and transferrin fusion protein by spacer optimization. Pharm Res 23, 2116-2121 (2006).
51. K. Sliepen, T. van Montfort, M. Melchers, G. Isik, R. W. Sanders, Immunosilencing a highly immunogenic protein trimerization domain J Biol Chem 290, 7436-7442 (2015).
52. S. Xia et al., A pan-coronavirus fusion inhibitor targeting the HR1 domain of human coronavirus spike. Sci Adv 5, eaav4580 (2019).
53. S. Xia et al., Inhibition of SARS-CoV-2 (previously 2019-nCoV) infection by a highly potent pan-coronavirus fusion inhibitor targeting its spike protein that harbors a high capacity to mediate membrane fusion. Cell Research 30, 343-355 (2020).
54. Y. W. Chenjian Gu, Huimin Guo, Yuanfei Zhu, Wei Xu, Yuyan Wang, Zhiping Sun, Xia Cai, Yutang Li, Jing Liu, Zhenghong Yuan, Rong Zhang, Qiang Deng, Di Qu, Youhua Xie, Potent antiviral effect of protoporphyrin IX and verteporfin on SARS-CoV-2 infection. bioRxiv doi: https://doi.org/10.1101/2020.04.30.071290, (2020).
55. X. Chi et al., A neutralizing human antibody binds to the N-terminal domain of the Spike protein of SARS-CoV-2. Science, (2020).
56. J. Lei, J. Frank, Automated acquisition of cryo-electron micrographs for single particle reconstruction on an FEI Tecnai electron microscope. Journal of structural biology 150, 69-80 (2005).
57. S. Q. Zheng et al., MotionCor2: anisotropic correction of beam-induced motion for improved cryo-electron microscopy. Nature methods 14, 331-332 (2017).
58. T. Grant, N. Grigorieff, Measuring the optimal exposure for single particle cryo-EM using a 2.6 A reconstruction of rotavirus VP6. eLife 4, e06980 (2015).
59. K. Zhang, Gctf: Real-time CTF determination and correction. Journal of structural biology 193, 1-12 (2016).
60. J. Zivanov et al., New tools for automated high-resolution cryo-EM structure determination in RELION-3. eLife 7, (2018).
61. D. Kimanius, B. O. Forsberg, S. H. Scheres, E Linclahl, Accelerated cryo-EM structure determination with parallelisation using GPUs in RELION-2. eLife 5, (2016).
62. S. H. Scheres, RELION: implementation of a Bayesian approach to cryo-EM structure determination. Journal of structural biology 180, 519-530 (2012).
63. S. H. Scheres, A Bayesian view on cryo-EM structure determination. Journal of molecular biology 415, 406-418 (2012).
64. A. Punjani, J. L. Rubinstein, D. J. Fleet, M. A. Brubaker, cryo SPARC: algorithms for rapid unsupervised cryo-EM structure determination. Nature methods 14, 290-296 (2017).
65. P. B. Rosenthal, R. Henderson, Optimal determination of particle orientation, absolute hand, and contrast loss in single-particle electron cryomicroscopy. Journal of molecular biology 333, 721-745 (2003).
66. S. Chen et al., High-resolution noise substitution to measure overfilling and validate resolution in 3D structure determination by single particle electron cryomicroscopy. Ultramicroscopy 135, 24-35 (2013).
67. L. G. Trabuco, E. Villa, K. Mitres, J. Frank, K. Schulten, Flexible fitting of atomic structures into electron microscopy maps using molecular dynamics Structure (London, England : 1993) 16, 673-683 (2008).
68. P. Emsley, B. Lohkamp, W. G. Scott, K. Cowtan, Features and development of Coot. Acta crystallographica. Section D, Biological crystallography 66, 486-501 (2010).
69. P. D. Adams et al., PHENIX: a comprehensive Python-based system for macromolecular structure solution. Acta crystallographica. Section D, Biological crystallography 66, 213-221 (2010).

1. ACE2 oligomer, wherein the ACE2 oligomer is formed by monomers, and each monomer comprises a soluble ACE2, a linker and an oligomerization motif, and wherein the ACE 2 oligomer comprises an ACE2 trimer.

2. The ACE2 oligomer of claim 1, wherein the ACE2 oligomer is an ACE2 trimer, and the oligomerization motif is a trimerization motif.

3. The ACE2 oligomer of claim 2, wherein the trimerization motif is a foldon motif or a three helix bundle motif.

4. The ACE2 oligomer of claim 1, wherein the linker is a flexible linker or a rigid linker.

5. The ACE2 oligomer of claim 1, wherein the linker comprises (EAAAK)3 (SEQ ID NO: 56) (AP)8 (SEQ ID NO: 57), (GGGGS)5 (SEQ ID NO: 49) or an amino acid sequence as set forth in SEQ ID NO: 40, 41, 42, 43, 44, 45, 46, 47, 48 or 50.

6.-7. (canceled)

8. The ACE2 oligomer of claim 1, wherein the soluble ACE2 comprises a sequence that is at least 90% identical to SEQ ID NO: 3, 51, 52, 53, 54 or 55.

9. A composition comprising the ACE2 oligomer of claim 1.

10. The composition of claim 9, wherein the composition is a pharmaceutical composition and comprises a pharmaceutically acceptable carrier.

11.-18. (canceled)

19. A method of treating or preventing coronavirus infection, comprising administering to a subject a therapeutically or prophylactically effective amount of the composition of claim 10.

10. A method of detecting coronavirus in a sample, comprising obtaining a sample, and contacting the sample with the ACE2 oligomer of claim 1.

21. The method of claim 20, wherein the coronavirus is SARS-CoV, SARS-CoV-2, SARSr-CoV, a mutant of SARS-CoV, a mutant of SARS-CoV-2 a mutant of SARSr-CoV and/or other coronaviruses having ACE2 as the cell entry receptor.

22. The method of claim 20, wherein the coronavirus is SARS-CoV, SARS-CoV-2, SARSr-CoV, a mutant of SARS-CoV, a mutant of SARS-CoV-2, a mutant of SARSr-CoV and/or other coronaviruses having ACE2 as the cell entry receptor.

23. The composition of claim 9, wherein the ACE2 oligomer is an ACE2 trimer, and the oligomerization motif is a trimerization motif.

24. The composition of claim 9, wherein the trimerization motif is a foldon motif or a three helix bundle motif.

25. The composition of claim 9, wherein the linker is a flexible linker or a rigid linker.

26. The composition of claim 9, wherein the linker comprises (EAAAK)3 (SEQ ID NO: 56) (AP)8 (SEQ ID NO: 57), (GGGGS)5 (SEQ ID NO: 49) or an amino acid sequence as set forth in SEQ ID NO: 40, 41, 42, 43, 44, 45, 46, 47, 48 or 50.

27. The composition of claim 9, wherein the soluble ACE2 comprises a sequence that is at least 90% identical to SEQ ID NO: 3, 51, 52, 53, 54 or 55.

28. The method of claim 19, wherein the ACE2 oligomer is an ACE2 trimer, the oligomerization motif is a foldon motif or a three helix bundle motif, and the linker comprises (EAAAK)3 (SEQ ID NO: 56) (AP)8 (SEQ ID NO: 57), (GGGGS)5 (SEQ ID NO: 49) or an amino acid sequence as set forth in SEQ ID NO: 40, 41, 42, 43, 44, 45, 46, 47, 48 or 50.

29. The method of claim 20, wherein the ACE2 oligomer is an ACE2 trimer, the oligomerization motif is a foldon motif or a three helix bundle motif, and the linker comprises (EAAAK)3 (SEQ ID NO: 56) (AP)8 (SEQ ID NO: 57), (GGGGS)5 (SEQ ID NO: 49) or an amino acid sequence as set forth in SEQ ID NO: 40, 41, 42, 43, 44, 45, 46, 47, 48 or 50.


SEQUENCE LISTING (72 sequences)

SEQ ID NO: 1 - protein, 805 aa, Homo sapiens [sequence omitted]

SEQ ID NO: 2 - protein, 740 aa, Homo sapiens
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 3 - protein, 598 aa, Homo sapiens
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 4 - DNA, 1968 bp, Artificial Sequence
  Artificial ACE2 monomer
cagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagacggcagcgaagcggcggcgaaagaagcggcggcgaaagaagcggcggcgaaagaagcggcggcgaaagaagcggcggcgaaaggaagcggctacatccccgaggcccccagggacggccaggcctacgtgaggaaggacggcgagtgggtgctgctgagcaccttcctgggcagc

SEQ ID NO: 5 - protein, 656 aa, Artificial Sequence
  Artificial ACE2 monomer
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADGSEAAAKEAAAKEAAAKEAAAKEAAAKGSGYIPEAPRDGQAYVRKDGEWVLLSTFLGS

SEQ ID NO: 6 - DNA, 1977 bp, Artificial Sequence
  Artificial ACE2 monomer
cagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagacggcagcgaagcggcggcgaaagaagcggcggcgaaagaagcggcggcgaaagaagcggcggcgaaagaagcggcggcgaaaggaagcggcgagatcgccgccatcaagcaggagatcgccgccatcaagaaggagatcgccgccatcaagtgggagatcgccgccatcaagcagggctacggc

SEQ ID NO: 7 - protein, 659 aa, Artificial Sequence
  Artificial ACE2 monomer
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADGSEAAAKEAAAKEAAAKEAAAKEAAAKGSGEIAAIKQEIAAIKKEIAAIKWEIAAIKQGYG

SEQ ID NO: 8 - DNA, 1950 bp, Artificial Sequence
  Artificial ACE2 monomer
cagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagacggtggaggaggttctggcggaggaggtagtggcggaggaggttcaggaggcggcggaagcggtggaggaggttctggctacatccccgaggcccccagggacggccaggcctacgtgaggaaggacggcgagtgggtgctgctgagcaccttcctg

SEQ ID NO: 9 - protein, 650 aa, Artificial Sequence
  Artificial ACE2 monomer
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADGGGGSGGGGSGGGGSGGGGSGGGGSGYIPEAPRDGQAYVRKDGEWVLLSTFL

SEQ ID NO: 10 - DNA, 1965 bp, Artificial Sequence
  Artificial ACE2 monomer
cagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagacggtggaggaggttctggcggaggaggtagtggcggaggaggttcaggaggcggcggaagcggtggaggaggttctggcgagatcgccgccatcaagcaggagatcgccgccatcaagaaggagatcgccgccatcaagtgggagatcgccgccatcaagcagggctacggc

SEQ ID NO: 11 - protein, 655 aa, Artificial Sequence
  Artificial ACE2 monomer
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADGGGGSGGGGSGGGGSGGGGSGGGGSGEIAAIKQEIAAIKKEIAAIKWEIAAIKQGYG

SEQ ID NO: 12 - DNA, 1869 bp, Artificial Sequence
  Artificial ACE2 monomer
cagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagacggtggaggaggttctggcggaggaggtagtggcggaggaggttcaggaggcggcggaagcggtggaggaggttct

SEQ ID NO: 13 - protein, 623 aa, Artificial Sequence
  Artificial ACE2 monomer
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADGGGGSGGGGSGGGGSGGGGSGGGGS

SEQ ID NO: 14 - DNA, 1968 bp, Artificial Sequence
  Artificial ACE2 monomer
cagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagacggcagcgccgaggccgccgccaaggaagccgctgctaaggaagccgctgccaaggaggccgccgccaaagaggccgccgctaaggccggctctggctacatccccgaggcccccagggacggccaggcctacgtgaggaaggacggcgagtgggtgctgctgagcaccttcctg

SEQ ID NO: 15 - protein, 655 aa, Artificial Sequence
  Artificial ACE2 monomer
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADGSAEAAAKEAAAKEAAAKEAAAKEAAAKAGSYIPEAPRDGQAYVRKDGEWVLLSTFL

SEQ ID NO: 16 - DNA, 1980 bp, Artificial Sequence
  Artificial ACE2 monomer
cagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagacggcggcggcggcagcgaggccgccgccaaggaagccgctgccaaggaagccgccgctaaagaggccgctgccaaggaagccgccgctaagggcggaggcggatctggctacatccccgaggcccccagggacggccaggcctacgtgaggaaggacggcgagtgggtgctgctgagcaccttcctg

SEQ ID NO: 17 - protein, 660 aa, Artificial Sequence
  Artificial ACE2 monomer
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADGGGGSEAAAKEAAAKEAAAKEAAAKEAAAKGGGGSGYIPEAPRDGQAYVRKDGEWVLLSTFL

SEQ ID NO: 18 - DNA, 1932 bp, Artificial Sequence
  Artificial ACE2 monomer
cagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagacggcagcgaggccgccgctaaagaggccgccgccaaggaagccgctgccaagggctctggctacatccccgaggcccccagggacggccaggcctacgtgaggaaggacggcgagtgggtgctgctgagcaccttcctg

SEQ ID NO: 19 - protein, 643 aa, Artificial Sequence
  Artificial ACE2 monomer
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADGSEAAAKEAAAKEAAAKGSYIPEAPRDGQAYVRKDGEWVLLSTFL

SEQ ID NO: 20 - DNA, 1947 bp, Artificial Sequence
  Artificial ACE2 monomer
cagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagacggcagcgaggccgccgccaaggaagccgccgccaaggaagccgctgctaaagaggccgctgccaagggctctggctacatccccgaggcccccagggacggccaggcctacgtgaggaaggacggcgagtgggtgctgctgagcaccttcctg

SEQ ID NO: 21 - protein, 648 aa, Artificial Sequence
  Artificial ACE2 monomer
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADGSEAAAKEAAAKEAAAKEAAAKGSYIPEAPRDGQAYVRKDGEWVLLSTFL

SEQ ID NO: 22 - DNA, 1977 bp, Artificial Sequence
  Artificial ACE2 monomer
cagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagacggatctgaggccgctgcaaaggaagccgccgctaaagaggccgccgctaaggaagccgctgccaaggaagccgccgccaaagaggccgccgccaagggcagcggctacatccccgaggcccccagggacggccaggcctacgtgaggaaggacggcgagtgggtgctgctgagcaccttcctg

SEQ ID NO: 23 - protein, 659 aa, Artificial Sequence
  Artificial ACE2 monomer
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADGSEAAAKEAAAKEAAAKEAAAKEAAAKEAAAKGSGYIPEAPRDGQAYVRKDGEWVLLSTFL

SEQ ID NO: 24 - DNA, 1992 bp, Artificial Sequence
  Artificial ACE2 monomer
cagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagacggcagcgaggccgctgccaaagaggccgccgccaaggaagccgctgcaaaggaagccgccgctaaagaggccgctgccaaggaggccgccgccaaggaagccgccgccaagggctctggctacatccccgaggcccccagggacggccaggcctacgtgaggaaggacggcgagtgggtgctgctgagcaccttcctg

SEQ ID NO: 25 - protein, 663 aa, Artificial Sequence
  Artificial ACE2 monomer
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADGSEAAAKEAAAKEAAAKEAAAKEAAAKEAAAKEAAAKGSYIPEAPRDGQAYVRKDGEWVLLSTFL

SEQ ID NO: 26 - DNA, 1959 bp, Artificial Sequence
  Artificial ACE2 monomer
cagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagacggatctgccccagcccctgcccctgcccctgctccagctcccgcccctgcccctgcccctgcccctgcccctgctcccggcagcggctacatccccgaggcccccagggacggccaggcctacgtgaggaaggacggcgagtgggtgctgctgagcaccttcctg

SEQ ID NO: 27 - protein, 653 aa, Artificial Sequence
  Artificial ACE2 monomer
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADGSAPAPAPAPAPAPAPAPAPAPAPAPGSGYIPEAPRDGQAYVRKDGEWVLLSTFL

SEQ ID NO: 28 - DNA, 1977 bp, Artificial Sequence
  Artificial ACE2 monomer
cagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagacggatctgcccctgcccctgctccagctcccgcccctgcccctgcccctgcccctgcccctgcccctgctcccgcccctgctccagcccctgcccccggcagcggctacatccccgaggcccccagggacggccaggcctacgtgaggaaggacggcgagtgggtgctgctgagcaccttcctg

SEQ ID NO: 29 - protein, 659 aa, Artificial Sequence
  Artificial ACE2 monomer
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADGSAPAPAPAPAPAPAPAPAPAPAPAPAPAPAPGSGYIPEAPRDGQAYVRKDGEWVLLSTFL

SEQ ID NO: 30 - DNA, 1986 bp, Artificial Sequence
  Artificial ACE2 monomer
cagtccaccattgaggaacaggccaagacatttttggacttctttaacatccaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagacggatctgcccctgcccctgctccagctcccgcccctgcccctgcccctgcccctgcccctgcccctgctcccgcccctgctccagcccctgcccccggcagcggctacatccccgaggcccccagggacggccaggcctacgtgaggaaggacggcgagtgggtgctgctgagcaccttcctgggcggcaga

SEQ ID NO: 31 - protein, 659 aa, Artificial Sequence
  Artificial ACE2 monomer
QSTIEEQAKTFLDFFNIQAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADGSAPAPAPAPAPAPAPAPAPAPAPAPAPAPAPGSGYIPEAPRDGQAYVRKDGEWVLLSTFL

SEQ ID NO: 32 - DNA, 1986 bp, Artificial Sequence
  Artificial ACE2 monomer
cagtccaccattgaggaacaggccaagtactttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacatacgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaatattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagacggatctgcccctgcccctgctccagctcccgcccctgcccctgcccctgcccctgcccctgcccctgctcccgcccctgctccagcccctgcccccggcagcggctacatccccgaggcccccagggacggccaggcctacgtgaggaaggacggcgagtgggtgctgctgagcaccttcctgggcggcaga

SEQ ID NO: 33 - protein, 659 aa, Artificial Sequence
  Artificial ACE2 monomer
QSTIEEQAKYFLDKFNHEAEDLFYQSSLASWNYNTNITEENVQNMNNAGDKWSAFLKEQSTYAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADGSAPAPAPAPAPAPAPAPAPAPAPAPAPAPAPGSGYIPEAPRDGQAYVRKDGEWVLLSTFL

SEQ ID NO: 34 - DNA, 1986 bp, Artificial Sequence
  Artificial ACE2 monomer
cagtccaccattgaggaacaggccaagtactttttggacaagtttaacgctgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagacggatctgcccctgcccctgctccagctcccgcccctgcccctgcccctgcccctgcccctgcccctgctcccgcccctgctccagcccctgcccccggcagcggctacatccccgaggcccccagggacggccaggcctacgtgaggaaggacggcgagtgggtgctgctgagcaccttcctgggcggcaga

SEQ ID NO: 35 - protein, 659 aa, Artificial Sequence
  Artificial ACE2 monomer
QSTIEEQAKYFLDKFNAEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADGSAPAPAPAPAPAPAPAPAPAPAPAPAPAPAPGSGYIPEAPRDGQAYVRKDGEWVLLSTFL

SEQ ID NO: 36 - DNA, 1986 bp, Artificial Sequence
  Artificial ACE2 monomer
cagtccaccattgaggaacaggccaagtactttttggacttctttaacatccaagccgaagacctgttctatcaaagttcacttgcttcttggaattataacaccaatattactgaagagaatgtccaaaacatgaataatgctggggacaaatggtctgcctttttaaaggaacagtccacatacgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaatattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagacggatctgcccctgcccctgctccagctcccgcccctgcccctgcccctgcccctgcccctgcccctgctcccgcccctgctccagcccctgcccccggcagcggctacatccccgaggcccccagggacggccaggcctacgtgaggaaggacggcgagtgggtgctgctgagcaccttcctgggcggcaga

SEQ ID NO: 37 - protein, 659 aa, Artificial Sequence
  Artificial ACE2 monomer
QSTIEEQAKYFLDFFNIQAEDLFYQSSLASWNYNTNITEENVQNMNNAGDKWSAFLKEQSTYAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADGSAPAPAPAPAPAPAPAPAPAPAPAPAPAPAPGSGYIPEAPRDGQAYVRKDGEWVLLSTFL

SEQ ID NO: 38 - DNA, 1986 bp, Artificial Sequence
  Artificial ACE2 monomer
cagtccaccattgaggaacaggccaagtactttttggacaagtttaacgctgaagccgaagacctgttctatcaaagttcacttgcttcttggaattataacaccaatattactgaagagaatgtccaaaacatgaataatgctggggacaaatggtctgcctttttaaaggaacagtccacatacgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaatattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagacggatctgcccctgcccctgctccagctcccgcccctgcccctgcccctgcccctgcccctgcccctgctcccgcccctgctccagcccctgcccccggcagcggctacatccccgaggcccccagggacggccaggcctacgtgaggaaggacggcgagtgggtgctgctgagcaccttcctgggcggcaga

SEQ ID NO: 39 - protein, 659 aa, Artificial Sequence
  Artificial ACE2 monomer
QSTIEEQAKYFLDKFNAEAEDLFYQSSLASWNYNTNITEENVQNMNNAGDKWSAFLKEQSTYAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADGSAPAPAPAPAPAPAPAPAPAPAPAPAPAPAPGSGYIPEAPRDGQAYVRKDGEWVLLSTFL

SEQ ID NO: 40 - protein, 29 aa, Artificial Sequence
  artificial linker
GSEAAAKEAAAKEAAAKEAAAKEAAAKGS

SEQ ID NO: 41 - protein, 31 aa, Artificial Sequence
  artificial linker
GSAEAAAKEAAAKEAAAKEAAAKEAAAKAGS

SEQ ID NO: 42 - protein, 35 aa, Artificial Sequence
  artificial linker
GGGGSEAAAKEAAAKEAAAKEAAAKEAAAKGGGGS

SEQ ID NO: 43 - protein, 19 aa, Artificial Sequence
  artificial linker
GSEAAAKEAAAKEAAAKGS

SEQ ID NO: 44 - protein, 24 aa, Artificial Sequence
  artificial linker
GSEAAAKEAAAKEAAAKEAAAKGS

SEQ ID NO: 45 - protein, 34 aa, Artificial Sequence
  artificial linker
GSEAAAKEAAAKEAAAKEAAAKEAAAKEAAAKGS

SEQ ID NO: 46 - protein, 39 aa, Artificial Sequence
  artificial linker
GSEAAAKEAAAKEAAAKEAAAKEAAAKEAAAKEAAAKGS

SEQ ID NO: 47 - protein, 28 aa, Artificial Sequence
  artificial linker
GSAPAPAPAPAPAPAPAPAPAPAPAPGS

SEQ ID NO: 48 - protein, 34 aa, Artificial Sequence
  artificial linker
GSAPAPAPAPAPAPAPAPAPAPAPAPAPAPAPGS

SEQ ID NO: 49 - protein, 25 aa, Artificial Sequence
  artificial linker
GGGGSGGGGSGGGGSGGGGSGGGGS

SEQ ID NO: 50 - protein, 25 aa, Artificial Sequence
  artificial linker
EAAAKEAAAKEAAAKEAAAKEAAAK

SEQ ID NO: 51 - protein, 598 aa, Homo sapiens
QSTIEEQAKTFLDFFNIQAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 52 - protein, 598 aa, Homo sapiens
QSTIEEQAKYFLDKFNHEAEDLFYQSSLASWNYNTNITEENVQNMNNAGDKWSAFLKEQSTYAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 53 - protein, 598 aa, Homo sapiens
QSTIEEQAKYFLDKFNAEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 54 - protein, 598 aa, Homo sapiens
QSTIEEQAKYFLDFFNIQAEDLFYQSSLASWNYNTNITEENVQNMNNAGDKWSAFLKEQSTYAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 55 - protein, 598 aa, Homo sapiens
QSTIEEQAKYFLDKFNAEAEDLFYQSSLASWNYNTNITEENVQNMNNAGDKWSAFLKEQSTYAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 56 - protein, 15 aa, Artificial Sequence
  linker (EAAAK)3
EAAAKEAAAKEAAAK

SEQ ID NO: 57 - protein, 16 aa, Artificial Sequence
  linker (AP)8
APAPAPAPAPAPAPAP

SEQ ID NO: 58 - protein, 5 aa, Artificial Sequence
  linker (EAAAK)n, wherein n can be any integer from 3 to 15
  misc_feature (1)..(5): repeat n times, wherein n can be any integer from 3 to 15
EAAAK

SEQ ID NO: 59 - protein, 2 aa, Artificial Sequence
  linker (AP)n, wherein n can be any integer from 8 to 22
  MISC_FEATURE (1)..(2): repeat n times, wherein n can be any integer from 8 to 22
AP

SEQ ID NO: 60 - protein, 15 aa, Artificial Sequence
  linker (GGGGS)3
GGGGSGGGGSGGGGS

SEQ ID NO: 61 - protein, 8 aa, Artificial Sequence
  linker (Gly)8
GGGGGGGG

SEQ ID NO: 62 - protein, 6 aa, Artificial Sequence
  linker (Gly)6
GGGGGG

SEQ ID NO: 63 - protein, 5 aa, Artificial Sequence
  linker GGGGS
GGGGS

SEQ ID NO: 64 - protein, 5 aa, Artificial Sequence
  linker (GGGGS)n (n=1, 2, 4)
  MISC_FEATURE (1)..(5): repeat n times, wherein n=1, 2, 4
GGGGS

SEQ ID NO: 65 - protein, 5 aa, Artificial Sequence
  linker (EAAAK)n (n=1-3)
  MISC_FEATURE (1)..(5): repeat n times, wherein n=1-3
EAAAK

SEQ ID NO: 66 - protein, 46 aa, Artificial Sequence
  linker A(EAAAK)4ALEA(EAAAK)4A
AEAAAKEAAAKEAAAKEAAAKALEAEAAAKEAAAKEAAAKEAAAKA

SEQ ID NO: 67 - protein, 12 aa, Artificial Sequence
  linker AEAAAKEAAAKA
AEAAAKEAAAKA

SEQ ID NO: 68 - protein, 5 aa, Artificial Sequence
  linker PAPAP
PAPAP

SEQ ID NO: 69 - protein, 2 aa, Artificial Sequence
  linker (Ala-Pro)n (10-34 aa)
  MISC_FEATURE (1)..(2): repeat n times, wherein the total length is 10-34 aa
AP

SEQ ID NO: 70 - DNA, 22 bp, Artificial Sequence
  SARS-CoV-2-N-F
ggggaacttctcctgctagaat

SEQ ID NO: 71 - DNA, 22 bp, Artificial Sequence
  SARS-CoV-2-N-R
cagacattttgctctcaagctg

SEQ ID NO: 72 - DNA, 20 bp, Artificial Sequence
  SARS-CoV-2-N-probe
  misc_feature (1)..(20): with a FAM on the 5′ end and a TAMRA on the 3′ end
ttgctgctgcttgacagatt